(19) United States
(12) Patent Application Publication
(10) Pub. No.: US 2019/0371460 A1
(43) Pub. Date: Dec. 5, 2019

(54) Detection and Display of Respiratory Rate Variability, Mechanical Ventilation Machine Learning, and Double Booking of Clinic Slots, System, Method, and Computer Program Product

(71) Applicant: Respivar LLC, McLean, VA (US)

(72) Inventor: Guillermo Gutierrez, McLean, VA (US)

(73) Assignee: Respivar LLV, McLean, VA (US)

(21) Appl. No.: 16/395,266

(22) Filed: Apr. 26, 2019

Related U.S. Application Data
(60) Provisional application No. 62/663,175, filed on Apr. 26, 2018.

(51) Int. Cl. G16H 40/63; G16H 50/30; A61M 16/00

(52) U.S. Cl. CPC G16H 40/63; G16H 50/30; A61M 16/0051; A61M 2205/584; A61M 2205/18; A61M 2205/3379; A61M 2202/048; A61M 2016/0027; A61M 2205/3368; A61M 2205/505; A61M 2230/04; A61M 2230/40; A61M 2016/0033

(57) ABSTRACT

A noninvasive of detecting patient-ventilator asynchrony that is easily adaptable to existing ventilator monitoring systems and provides timely and actionable information on the degree of patient asynchrony both during invasive and non-invasive ventilation. Capture, analysis or display of, frequency spectra and the use of a measure of spectral organization, such as H1/DC, allows for both manual and automatic adjustment of a ventilators to prevent or correct patient-ventilator asynchrony via interventions. Embodiments use artificial intelligence or machine learning to predict interventions predicted to result in positive outcomes, based on analysis of a large number of epochs, captured by an electronic monitor of a mechanical ventilator, where the monitor continuously monitors, captures and transfers, epochs of data for aggregated machine learning analysis, of such epochs associated with positive outcomes. Scheduling processes that seek to overbook or double book to overcome negative effects of no shows, on clinician productivity in a medical setting.

### BACKGROUND OF THE DISCLOSURE

### Technical Field of the Disclosure

The disclosure relates generally to medical devices, and more particularly to ventilator medical systems.

### Related Art

Asynchronous events occur during mechanical ventilation when a patient's intrinsic respiratory rhythm fails to entrain to machine inflation or when ventilatory support is inadequate to meet the patient's requirements. Patient-ventilator asynchrony is a condition that affects a significant proportion of patients undergoing mechanical ventilation. It may be present either at the beginning of inspiration (trigger asynchrony), when the inspiratory efforts of the patient and the ventilator are out of phase. It also may be present during expiration should the inspiratory flow provided by the ventilator stop before or after the patient's own inspiratory effort. Patient-ventilator asynchrony is a common occurrence in mechanically ventilated patients, in particular those with acute or severe lung injury. Poorly synchronized patients may develop respiratory muscle fatigue, remain on mechanical ventilation longer and appear to have worse outcomes. As used herein patient includes any subject, and is not limited to humans.

Appropriate ventilatory support depends to a great extent on the reliable recognition of ventilator asynchrony; however, this is not a simple task. Perhaps the most reliable method presently available to detect asynchrony is the placement of a balloon catheter in the esophagus to measure intra-thoracic pressure changes during the breath cycle. Electromyography also has been used to assess asynchrony by comparing ventilatory muscle electrical activity to the initiation of ventilator-delivered inspiratory flow. Both methods have the obvious disadvantage of being invasive and not well tolerated by many patients, in particular those who are alert and awake. Further, they are relatively complex and require considerable operator experience. Non-invasive methods to establish the degree of patient-ventilator synchronicity have been proposed as possible alternatives to electromyography and measures of intrathoracic pressures. Perhaps the method with the widest clinical acceptance is the asynchrony index (AI) initially described by Varon J, Fromm R, Rodarte J, Reinoso M. “Prevalence of patient-ventilator asynchrony in critically ill patients.” Chest 1994; 106:141S. Although useful as a research tool, the AI method is not easily applied to monitoring patient-ventilator asynchrony since it is laborious and operator dependent.

While assessing the incidence of patient-ventilator asynchrony during mechanical ventilation, one study found asynchrony in approximately 25% of patients. Thille A W, Rodriguez P, Cabello B, Lellouche F, Brochard L., “Patient-ventilator asynchrony during assisted mechanical ventilation.” Intensive Care Med. 2006; 32:1515-1522. This condition was associated with significantly longer duration of mechanical ventilation. In a subsequent study they eliminated ineffective triggering in two-thirds of asynchronous cases by decreasing pressure support or inspiratory duration. More recently, the use of a neurally adjusted ventilator assist to control the timing and pressure of assisted delivery has been shown to decrease asynchrony by reducing triggering and cycling delays. Examples of two such studies are described in Spahij a J, de Marchie M, Albert M, Bellemare P, Delisle S, Beck J, Sinderby C., “Patient-ventilator interaction during pressure support ventilation and neutrally adjusted ventilatory assist.” Crit. Care Med. 2010; 38:518-526; and Terzi N, Pelieu I, Guittet L, Ramakers M, Seguin A, Daubin C, Charbonneau P, du Cheyron D, Lofaso F. “Neurally adjusted ventilatory assist in patients recovering spontaneous breathing after acute respiratory distress syndrome: Physiological evaluation.” Crit. Care Med. 2010; 38:1830-1837.

Several noninvasive methods have been developed to automate the evaluation of asynchrony detection. These methods analyze airway signals searching for anomalies indicative of ineffective patient triggering (IT). Mulqueeny Q, Ceriana P, Carlucci A, Fanfulla F, Delmastro M, Nava S. “Automatic detection of ineffective triggering and double triggering during mechanical ventilation.” Int Care Med 2007; 33:2014-2018 proposed applying a noise filter and an unintentional leak compensation algorithm to the flow and pressure curves, followed by the calculation of the first and second derivatives of the flow signal. They tested their method in twenty mechanically ventilated patients and found 91% sensitivity and 97% specificity when compared to the manually derived AI.

Cuvelier A, Achour L, Rabarimanantsoa H, Letellier C, Muir J-F, Fauroux B. “A noninvasive method to identify ineffective triggering in patients with noninvasive pressure support ventilation.” Respiration 2010; 80:198-206, developed a complex algorithm that analyzed phase portraits, a geometrical depiction of temporal changes in patient-ventilator interaction. They were able to identify 95% of all IT efforts when comparing the results of this method to esophageal tracings in fourteen children with cystic fibrosis on non-invasive ventilation.

Chen C W, Lin W C, Hsu C H, Cheng K S, Lo C S. “Detecting ineffective triggering in the expiratory phase in mechanically ventilated patients based on airway flow and pressure deflection: feasibility of using a computer algorithm.” Crit. Care Med. 2008; 36:455-461, developed a computerized algorithm based on small deflections of the flow and pressure signals during the expiratory phase of ventilation. The algorithm detected IT with high sensitivity and specificity in fourteen ventilated patients. This method has the disadvantage of detecting only one type of patient-ventilator interaction.

Younes M, Brochard L, Grasso S, Kun J, Mancebo J, Ranieri M, Richard J C, Younes H. “A method for monitoring and improving patient: ventilator interaction.” Intensive Care Med. 2007; 33:1337-1346, monitored patient-ventilator interaction with a proprietary system that generates a signal mimicking respiratory muscle pressure output. The signal was derived from the equation of motion of the respiratory system using improvised values for resistance and elastance. This method could detect 80% of IT efforts when applied to airway signal tracings from 21 mechanically ventilated patients.

Aliasing of the airway signal with background noise may interfere with the ability of some of these methods to distinguish small deflections indicative of wasted inspiratory effort. Moreover, they may fail to identify conditions in which ventilatory support during inspiration is not sufficient to meet ventilatory requirements. Although apparently synchronous, this situation results in increased work of breathing from patient generated negative pressure efforts.

What is needed is an improved solution that overcomes the shortcomings of conventional solutions.

### SUMMARY

The disclosure sets forth various exemplary embodiments of systems, methods, and/or medical apparatuses including but not limited to: provide a method and system to detect respiratory asynchrony.

According to one example embodiment the medical device can include a feature to provide a non-invasive method and system to detect respiratory rate variability.

According to one example embodiment the medical device can include a feature to provide a method and system to detect respiratory rate variability. that does not require considerable operator experience.

According to one example embodiment the medical device can include a feature to provide a method and system to detect respiratory rate variability that allows appropriate, synchronous ventilatory support.

According to one example embodiment the medical device can include a feature to provide a method of detecting patient-ventilator asynchrony that comprises obtaining a signal representative of respiratory movement of a patient; modifying the signal in accordance with a Fourier transform to obtain a frequency spectrum of the signal; obtaining a measure of spectral organization of the obtained frequency spectrum; and providing an output signal representative of the measure.

According to one example embodiment the medical device can include a feature to provide a system for detecting patient-ventilator asynchrony that comprises a ventilator; a sensor operatively connected to an air output of the ventilator for providing an output representative of a characteristic of the air output; a data acquisition unit operatively connected to collect data representative of the sensor output to obtain a signal representative of respiratory movement of a patient; and a processor unit operatively connected to the data acquisition unit structured, and arranged to perform a Fourier transform of the collected data, to determine a measure of spectral organization of the transformation of the collected data, and to output a signal representative of the measure of spectral organization.

### BRIEF DESCRIPTION OF THE DRAWINGS

The accompanying drawings, which are included to provide a further understanding and are incorporated in and constitute a part of this specification, illustrate exemplary, and nonlimiting embodiments and together with the description serve to explain the principles disclosed herein. In the drawings, like reference numbers may indicate substantially similar, equivalent, or exemplary elements, and the left most digits in the corresponding reference number indicate the drawing in which an element first appears.

The subject matter disclosed herein is particularly pointed out and distinctly claimed as set forth in the claims at the conclusion of the specification. The foregoing and other objects, features and advantages will be apparent from the following detailed description taken in conjunction with the accompanying drawings, of which:

FIG. 1 is a schematic block diagram of an embodiment of a system for detecting respiratory asynchrony in accordance with an exemplary embodiment of the present invention;

FIGS. 2A and 2B are representative graphs of frequency spectra and corresponding H1/DC values are shown in FIGS. 2C and 2D, according to an exemplary embodiment of the present invention;

FIGS. 3A and 3B are additional representative graphs of frequency spectra and corresponding H1/DC values are shown in FIGS. 3C and 3D, according to an exemplary embodiment of the present invention;

FIGS. 4A, 4C, 4E include graphs and FIGS. 4B, 4D, 4F show corresponding frequency spectra for airway flow data, according to an exemplary embodiment of the present invention;

FIG. 5 is a graph illustrating representative data correlating H1/DC values with patient-ventilator asynchrony, according to an exemplary embodiment of the present invention;

FIGS. 6A-6D are representative graphs of frequency spectra for various identified types of frequency spectra, according to an exemplary embodiment of the present invention;

FIG. 7 is a representative graph correlating frequency spectra types and H1/DC, according to an exemplary embodiment of the present invention;

FIG. 8 is a generalized schematic diagram of parameters influencing the frequency of respiration, according to an exemplary embodiment of the present invention;

FIG. 9 schematically illustrates an exemplary ventilator feedback system, according to an exemplary embodiment of the present invention;

FIG. 10 depicts an exemplary embodiment of a diagram illustrating a fast Fourier transform between the time domain and frequency domain and the zero frequency peak DC voltage level as well as the H1 first harmonic peak, the ratio of which is illustrated, according to an exemplary embodiment;

FIG. 11 depicts an exemplary embodiment of a diagram illustrating a top level functional block diagram of an example continuous respiration monitor, according to an exemplary embodiment of the present invention;

FIGS. 12A, 12B and 12C depict exemplary embodiments of diagrams, illustrating an exemplary front view, exemplary side and exemplary back view of an example box and stand setup of the example continuous respiration monitor including a photograph image of the front of the box powered on mounted in a stand, and a photograph image of the back of the box mounted in an example stand, respectively, according to an exemplary embodiment of the present invention;

FIG. 13 depicts an exemplary embodiment of a raspberry pi 2 micro controller as could be used in one exemplary embodiment of monitor, according to an exemplary embodiment of the present invention;

FIG. 14 depicts an exemplary embodiment of a raspberry pi 3 micro controller as could be used in one exemplary embodiment of monitor, according to an exemplary embodiment;

FIG. 15 depicts an exemplary embodiment of a diagram illustrating an example power block diagram overview of one example continuous respiration monitor device, according to an exemplary embodiment of the present invention;

FIG. 16 depicts an exemplary embodiment of a diagram illustrating example data processing functions of the example continuous respiration monitor device, according to an exemplary embodiment of the present invention;

FIG. 17 depicts an exemplary display output with exemplary color sectors and an exemplary trend button, according to an exemplary embodiment;

FIG. 18 depicts an exemplary flow diagram of an exemplary objective, function, and specification data processing exemplary block diagram, according to an exemplary embodiment;

FIG. 19 depicts an exemplary embodiment of an exemplary composite dashboard according to an exemplary embodiment of the present invention;

FIG. 20 depicts an exemplary embodiment of an exemplary schematic diagram showing various machine learning algorithms, according to an exemplary embodiment of the present invention;

FIG. 21 depicts an exemplary embodiment of exemplary black bars showing the percentage of exemplary synchronous, variable breathing and asynchronous epochs associated with the use of i.v. propofol, according to an exemplary embodiment;

FIG. 22 depicts an image of an exemplary embodiment of a monitor as shown in block diagram of FIG. 11, illustrated coupled to a mechanical ventilator, according to an exemplary embodiment of the present invention;

FIGS. 23A-23G depict various exexmplary Examples of Epoch Classification, according to an exemplary embodiment.

FIG. 24 depicts an exemplary embodiment of a diagram illustrating example formatting of data referenced in an accompanying appendix, according to an exemplary embodiment of the present invention;

FIG. 25 depicts an exemplary embodiment of a diagram illustrating an example chart graphing average vs. difference for ration H1/DC accuracy testing, graphing differences between example Mbed and MATLAB H1/DC results, according to an exemplary embodiment of the present invention;

FIG. 26 depicts an exemplary computer system environment and/or platform, according to one exemplary embodiment;

FIG. 27 depicts an exemplary Statistical Simulation Model, according to an exemplary embodiment;

FIG. 28 depicts an exemplary model dashboard for a particular exemplary clinician (Dr. Panjrath), according to an exemplary embodiment;

FIG. 29 depicts an exemplary model dashboard illustrating, for an example, exemplary overbooking occurs when more than 11 slots are needed to accommodate patients who showed up, according to an exemplary embodiment;

FIG. 30 depicts an exemplary chart of exemplary simulation results graphing an effect on arrived RPV of double booking (DB) one Medicaid slot, as is demonstrated, No patient group was negatively affected, and Same results were observed with managed care and Medicare, according to an exemplary embodiment;

FIG. 31 depicts an exemplary block diagram of exemplary Proposed scheduling process, according to an exemplary embodiment; and

FIG. 32 depicts an exemplary project management GANNT chart of an exemplary rollout of an exemplary project to test efficacy of an exemplary double booking project in an exemplary clinic, according to an exemplary embodiment.

### DETAILED DESCRIPTION OF VARIOUS EXEMPLARY EMBODIMENTS

It is important to note that the embodiments disclosed are only examples of the many advantageous uses of the innovative teachings herein. In general, statements made in the specification of the present application do not necessarily limit any of the various claims. Moreover, some statements may apply to some exemplary features but not to others. In general, unless otherwise indicated, singular elements may be in plural and vice versa with no loss of generality. In the drawings, like numerals refer to like parts through several views.

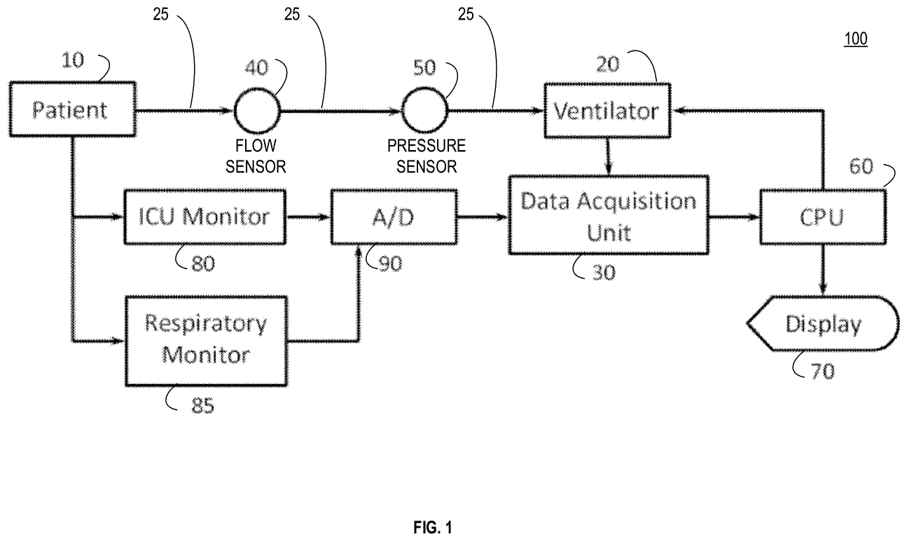

FIG. 1 is a schematic block diagram of an embodiment of a system for detecting respiratory asynchrony in accordance with the present invention. Referring to FIG. 1, in an exemplary embodiment, a patient 10 is coupled to a ventilator 20 via, for example, one or more flexible tubes or hoses 25 (not shown, but represented by the logical arrows between elements 10, 40, 50, and 20. A data acquisition system 30 acquires data such as air flow data from air flow sensor 40, air pressure data from pressure sensor 50, and/or data from any other data source (e.g., sensor) such as respiratory monitor 85, or optional intensive care unit 80. Preferably such other data sources or sensors are capable of sensing any periodic signal produced by breathing, for example, motion of the chest and abdomen or air temperature changes measured with a thermistor. As shown in the illustrative embodiment of FIG. 1, the air flow sensor 40 senses the air flow provided to patient 10 via hose 25. Also as shown in FIG. 1, pressure sensor 50 senses the pressure of the air in the hose 25.

In a preferred embodiment, the ventilator can comprise a SERVO-I ventilator provided by MAQUET Critical Care A B, Solna, Sweden. This exemplary ventilator includes a built-in data acquisition system that was used as the data acquisition system 30 shown in FIG. 1. It is not necessary that a ventilator have a built-in data acquisition system. For example, a ventilator without a built-in data acquisition system could be used together with data being monitored in analog form and digitized by a computer.

In the FIG. 1 embodiment, air flow was monitored continuously using SERVO-I ventilator built-in data acquisition system, and sampled the air flow at 30 Hz, a sampling rate exceeding the Nyquist criterion for respiratory signals. Obviously, signals can be sampled at any rate above the Nyquist rate for the signal of interest. In addition, other ventilation parameter could be collected, such as, the dynamic compliance (Cdyn=VT/[end inspiratory pressure—PEEP]); and 3) the inspiratory resistance (Ri=[Peak pressure—plateau pressure]/end inspiratory flow). Moreover, while the analysis in accordance with a preferred embodiment of the present invention focuses on air flow, and pressure, any periodic physiological signals produced during human ventilation, whether or not the individual is undergoing mechanical ventilation can be used. This allows for use with invasive and non-invasive ventilation, as well as with equipment used for sleep apnea. Possible exemplary signals include airway flow, airway pressure, tidal volume, measurements of flow made with, for example, thermistors placed near the nose or mouth, measurement of rib or abdominal expansion movements made during respiration and measured by any method, including plethysmography. In other words, anything that produces a periodic signal related to respiratory movements can used to produce a spectrum for analysis in accordance with embodiments of the present invention.

Also shown in the Figure embodiment is an ICU monitor 80. An exemplary ICU monitor can be TRAM® Multi-Parameter Module, GE Healthcare Bio-Sciences Corp., Piscataway, N.J., USA. In the illustrated embodiment, the ICU monitor monitors O2 saturation (SpO2), via, e.g., measured by pulse oximetry; arterial blood pressure from an arterial line; and heart rate from one electrocardiographic lead. These signals were sampled at 30 Hz rate from the analog output port of the ICU monitor (e.g., hemodynamic monitor) 80. In the illustrate embodiment being discussed here, an analog-to-digital converter 90 sampled the analog output of the ICU monitor 80 at a 30 Hz rate. One exemplary analog-to-digital converter can be, for example, a DI148U A/D, provided by DATAQ Instruments, Inc. Akron, Ohio, USA. It will be recognized by those skilled in the art that while individual systems are shown in FIG. 1, they need not be individual and can be integrated. In addition, the analog-to-digital converter need not be a separate subsystem and can be integrated into a monitoring device, into a data acquisition device, or into the CPU.

Referring to the illustrative embodiment of FIG. 1, a CPU 60 receives data acquired from the ventilator 20 and the ICU monitor 80. The CPU 60 can be a laptop, a dedicated processor, a controller, or any suitable processor capable of obtaining data and performing analysis in accordance with the present invention.

In one exemplary embodiment, the CPU 80 modified the air flow data by setting all inspiratory (positive) values to zero producing a periodic signal that included only the expiratory (negative) portion of airway flow. One skilled in the art will recognize that there are other suitable approaches, such as performing a FFT on the complete signal. The CPU 80 applied the Fast Fourier Transform, which is well known those skilled in the art to the modified air flow data. P. Duhamel, Vetterli M., “Fast Fourier transforms: a tutorial review and a state of the art,” Signal Processing 1990; 19: 259-299 provides one exemplary description of this signal processing technique. In one embodiment the data segments include 4096 consecutive signal samples, because the method used blocks of 2n samples. Each data segment comprised approximately 2.3 minutes of observation and provided sufficient information to compute a distinct frequency spectrum. The CPU 80 performed this procedure at 2.5 minute intervals, generating a total of 48 spectra during a two-hour observation period. A ratio of the first harmonic peak amplitude (H1) to the amplitude of the zero frequency or DC component (H1/DC ratio) was calculated for each spectrum. The AI was computed from the flow and pressure curves corresponding to the 2.3 minutes of observation to produce one spectrum.

The exemplary data shown in the accompanying figures was tested for equality of means conducted with Student's t-test with Bonferroni's correction for multiple comparisons. One-way ANOVA with post-hoc Tukey HSD test was used to compare multiple means from independent samples. The Fisher exact probability test with Pearson's correction was used to test for differences in categorical variables. The relationship between dependent variables was determined with linear regression analysis. The figures are shown as mean±SD. A p<0.05 was considered significant.

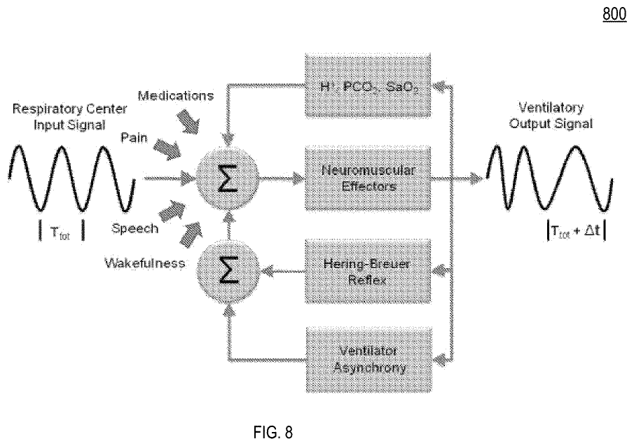

FIG. 8 is a schematic diagram illustrating some exemplary frequency components of a ventilator signal, such as the signal processed in accordance with embodiments of the present invention. The precise cellular and molecular mechanisms that generate and modulate respiratory rhythm remain unknown, it is useful to think of an idealized respiratory center input signal as having a nearly constant periodicity shown here as the time taken to complete one full breath cycle, TTot. Cortical inputs, among them the degree of alertness, speech, pain, etc, modify the respiratory center input signal as it activates the neuromuscular effectors to produce a ventilatory output signal. The periodicity of this output signal is no longer constant, as TTot is now altered by variations in time (Δt) that may have either positive or negative values from breath to breath. Feedback loops, among them mechanical output of respiratory muscles, peripheral chemoreceptors, and the Hering-Breuer mechanoreceptor reflex modulate the respiratory control of breathing. Under normal conditions At is small, imparting the respiratory cycle its inherent timing variability. Mechanical ventilation adds an additional feedback control that modulates breathing pattern. It is our hypothesis that asynchronous events during mechanical ventilation modify the Hering-Breuer reflex, increasing At on a breath-by-breath basis and greatly augmenting TTot variability. These changes in TTot are difficult to detect from direct examination of airway signal tracings.

Embodiments of the present invention use a parameter, H1/DC, to characterize the frequency spectral pattern. Such a parameter represents a measure of spectral organization. Other parameters reflective of spectral organization can be used, such as, for example, H2/DC, H3/DC, H2/H1, coherence function etc. In other words, any pattern recognition scheme to determine the degree of peak sharpness can be used as an indication of spectral organization; and thus to indicate the presence of asynchrony. While the following discussion of one exemplary embodiment focuses on H1/DC, it is not intended to limit the invention to a specific measure of spectral organization, or disorganization.

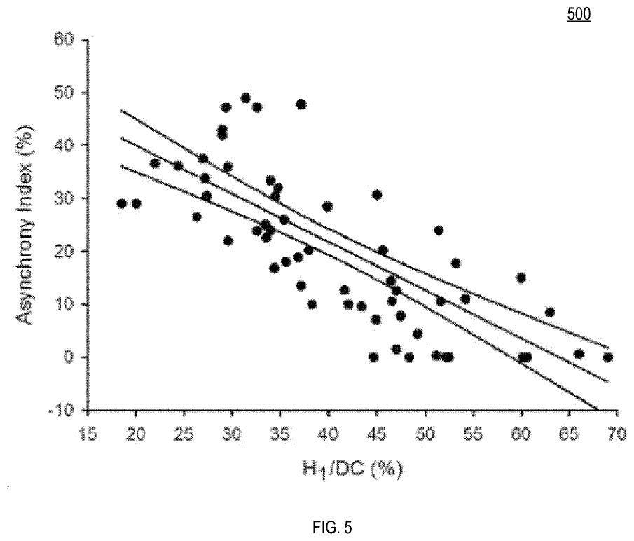

In embodiments using H1/DC, greater H1/DC values corresponded to more organized spectral patterns and were usually seen in what appeared to be better synchronized patients. These observations were confirmed by assessing patient-ventilator asynchrony using the AI method in which an inverse correlation was found between AI and H1/DC. The AI method lacks sensitivity and is prone to observer associated errors, which may explain the dispersion noted when correlating AI to H1/DC, such as shown in FIG. 5. In addition, the strength of the correlation (r=0.74) supports the association of decreases in H1/DC with greater patient-ventilator asynchrony.

The use of the parameter H1/DC to characterize the frequency spectral pattern has a solid physiological foundation. For example, the DC amplitude corresponds to mean expiratory flow and the H1 peak is located at the frequency corresponding to the average respiratory rate for the period of observation. In embodiments of the present invention, a H1/DC of approximately 45%, and in particular 43%, is used to differentiate organized from disorganized spectral patterns with a high degree of sensitivity and specificity.

In view of the above, embodiments of the present invention utilize H1/DC as a non-invasive parameter of asynchrony, with H1/DC of approximately less than 45% (e.g., 43%) serving as cutoff for patients having difficulties entraining to the ventilator. While it is possible that different ventilatory modes could alter the frequency spectra of airway signals; an embodiment discussed herein substantially eliminates that possibility by using only the expiratory portion of the flow signal. As modified, this flow signal reflects only patient related physiology, be it passive expiration or forced expiratory efforts, and is independent of the method employed to insufflate the lungs. This modification is, however, not essential to the present invention and other embodiments without this modification can be used.

In accordance with embodiments of the present invention, spectral patterns are determined and can be displayed on a display 70 for modification of ventilation. Obviously, a totally disorganized pattern (Type 1) indicates severe asynchrony, a condition known to be detrimental to patients, and indicates that ventilation adjustment is needed. On the other hand, normal physiological systems carry some degree of noise32 and a highly organized spectral pattern (Type 4) also may not be desirable, as it could indicate other conditions that adversely affect outcome33 such as the excessive use of sedatives and neuromuscular blockade.

The Fourier frequency analysis utilized in embodiments of the present invention provides an indication of breath-by-breath alterations in TTot. The exemplary embodiments herein use TTot as the main determinant of spectral pattern. In other words, as breath-by-breath variation in TTot increases, airflow frequency spectra shift from a highly organized to a disorganized pattern. Applying a Fourier frequency analysis to the data provides a correlation between such changes and patient-ventilator asynchrony and allows detection of patient-ventilator asynchrony. Changes in TTot are difficult to detect when examining the time dependent airflow signal but are apparent when presented as a frequency spectrum. As discussed below, different spectral patterns correlated with the degree of sedation and patient-ventilator asynchrony. Patients who were well synchronized with the ventilator displayed a spectral pattern composed of sharp Gaussian-shaped peaks, monotonically spaced at frequency multiples of the respiratory rate. This spectral pattern indicates a nearly perfect patient-ventilator coupling. As patient-ventilator asynchrony increases, the frequency spectra evolves into a more chaotic pattern, one in which the bandwidth of the Gaussian-shaped peaks widens, their amplitudes decrease, and higher frequency harmonics disappear.

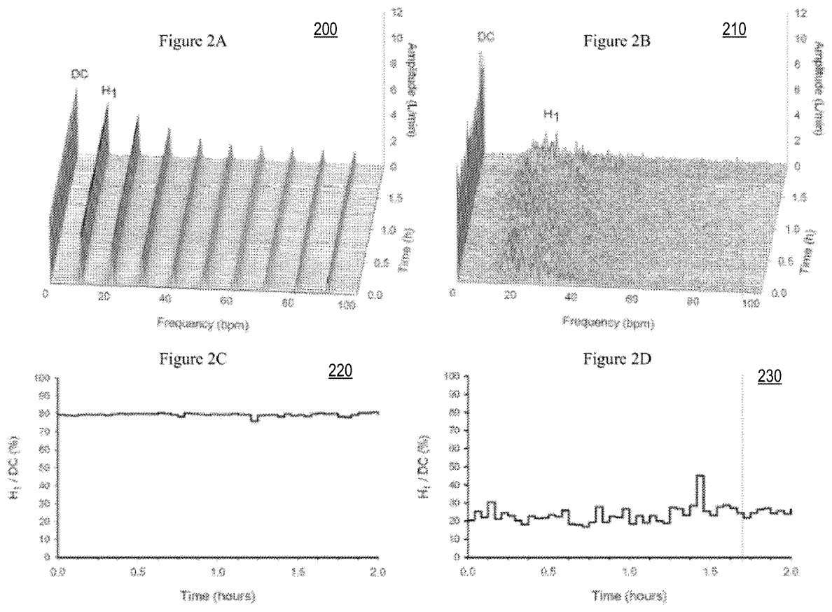

FIG. 2 shows a frequency spectra corresponding to two patients who, as judged by the AI, had very different levels of asynchrony. The 48 spectra obtained during the two-hour observation period are arranged in the form of a staggered ensemble beginning with the first spectrum. Patient A was a 63 y/o woman being ventilated through an endotracheal tube with clinical and radiological signs of brain death. The patient was perfectly coupled to the ventilator with an AI of zero. The spectra are characterized by sharp Gaussian-like peaks located at multiples of the fundamental frequency, or mean respiratory rate of 10.0±0.0 breaths per minute (bpm). Each spectrum repeats almost exactly every 2.5 minutes. Patient B was a 68 y/o woman ventilated through a tracheostomy with several failed attempts at weaning She was fully awake during the period of observation and appeared to be poorly synchronized with the ventilator, as evidenced both by direct observation and an AI of 46%. The spectra are highly disorganized with broadly based and poorly defined H1 peaks centered at the respiratory rate of 20.2±4.6 bpm. The spectra also lack subsequent harmonic peaks. Both spectral ensembles had measurable DC components corresponding to the mean of the expiratory flow signal (5.2±0.1 L/min for patient A; 7.3±0.8 L/min for patient B, respectively; p<0.01). FIG. 2 also shows changes in the H1/DC ratio corresponding to the spectral ensembles. During the two-hour observation period this ratio was approximately constant at 79.5±0.8% for patient A, but was lower and had greater variability for patient B at 23.8±4.5% (p<0.001).

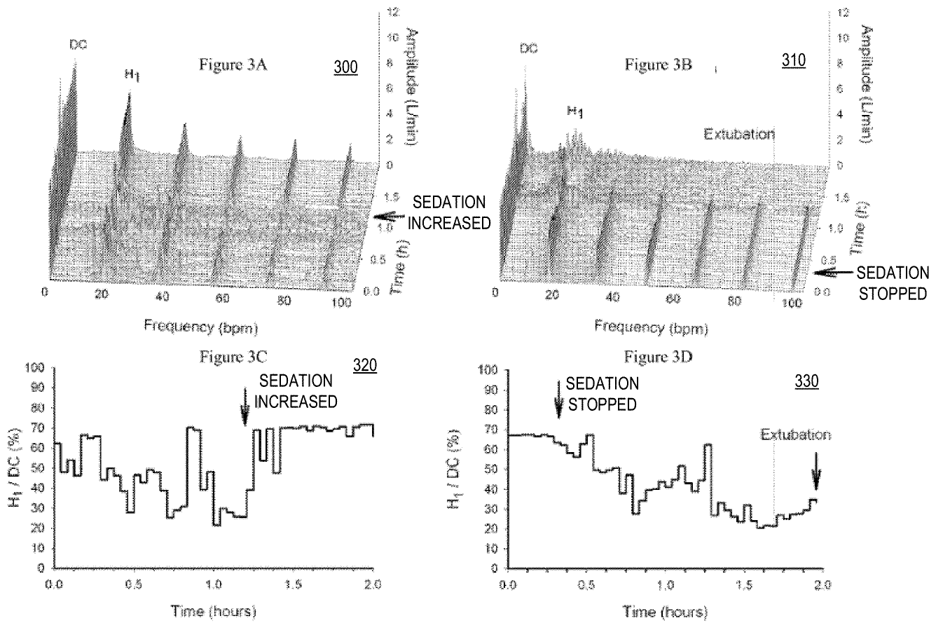

The frequency ensembles from most of the observed patients showed spectral patterns falling somewhere between those of FIG. 2, at times even displayed characteristics of both patterns during the two hour observation period. This is illustrated in FIG. 3 where an 88 y/o woman with congestive heart failure (Patient C) became increasingly uncomfortable on the ventilator during the period of observation. This was associated with decreasing spectral organization and a decline in the H1/DC ratio from an initial value of 62% to a nadir of 22% after one hour of observation. As the i.v. dose of the sedative fentanyl was doubled, the frequency spectra reverted to the Gaussian-peak pattern with a rise in H1/DC to 70%. A different spectral progression was seen when monitoring Patient D, a 52 y/o man admitted to the ICU following coronary artery bypass surgery. The patient's i.v. sedation was discontinued shortly after beginning data recording and he was extubated two hours later. Expiratory flow spectra initially showed a distinct Gaussian-peak pattern, with H1 located at respiratory rate of 16 bpm and H1/DC ratio of 67%. The patient's spectra became progressively disorganized as sedation wore off, with decreases in H1/DC ratio to 20%-30% just prior to extubation.

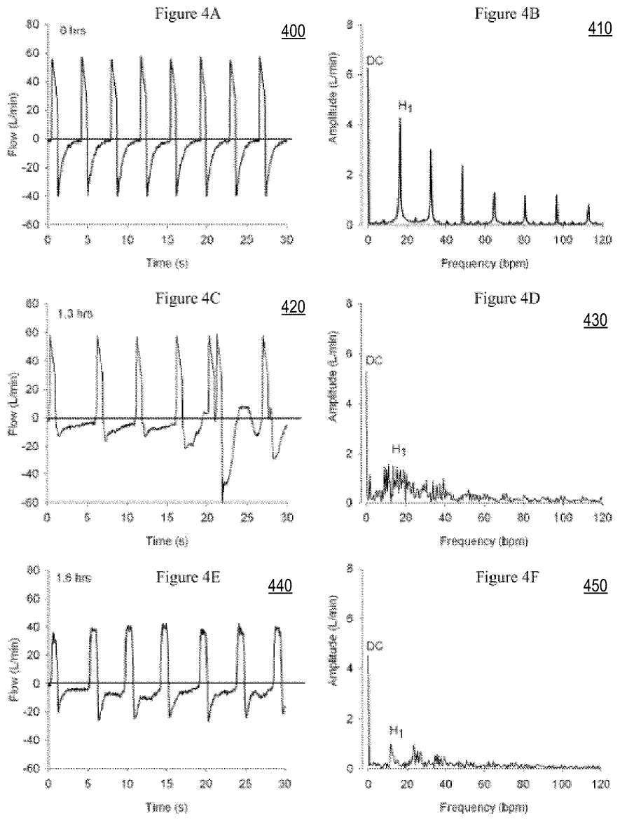

FIG. 4 shows the complete flow signals for patient D of FIG. 3 measured at times zero, 1.3 hours and 1.6 hours, along with the frequency spectra of the expiratory flow. Airway flow at time zero shows near perfect patient-ventilator synchronization corresponding to a highly organized spectrum having sharp Gaussian peaks at frequencies multiple of the respiratory rate (16.3 bpm; H1/DC=67%). At 1.3 hours the flow signal shows signs of asynchrony, characterized by double triggering. This was associated with an extremely disorganized frequency spectrum with a broadly based H1 centered at 14.5 bpm, loss of subsequent harmonic peaks and H1/DC of 29%. The expiratory flow signal at 1.6 hours shows more subtle signs of asynchrony, such as increases in end-inspiratory flow and the initiation of ventilator-driven inspiratory effort prior to end-expiration, with the possible development of dynamic hyperinflation. Although at first glance this flow signal appears to be more regular than the previous one, subtle breath-by-breath alterations in TTot resulted also in a disorganized spectral pattern with H1/DC of only 21%.

In addition to the above, the AI from the airway signals and H1/DC from the expiratory flow frequency spectrum at observation times 0, 30, 60, 90 and 120 minutes were obtained, and their average values calculated. FIG. 5 shows individual mean AI plotted as a function of the corresponding mean H1/DC for all patients in the study (n=66). There was a strong inverse relationship between AI and H1/DC (AI=58.2-0.9 H1/DC; r2=0.54, p<0.001) supporting the notion that decreases in H1/DC are associated with increases in patient-ventilator asynchrony.

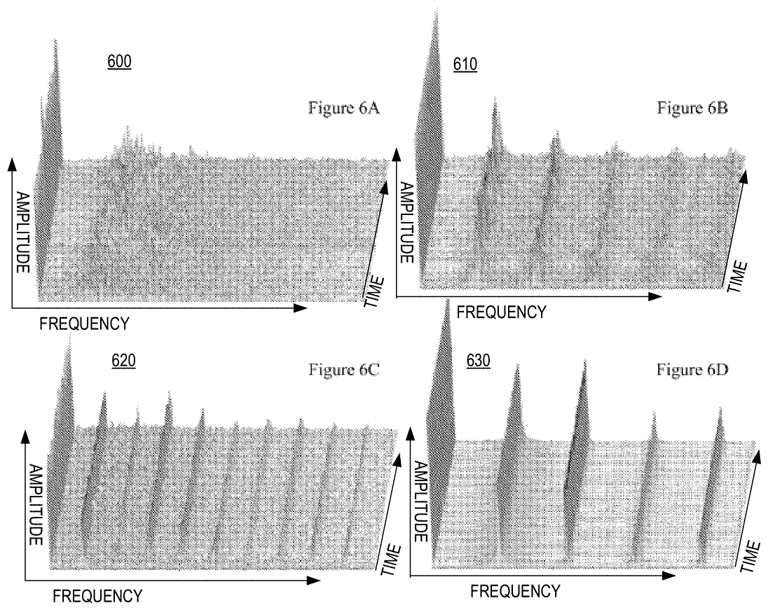

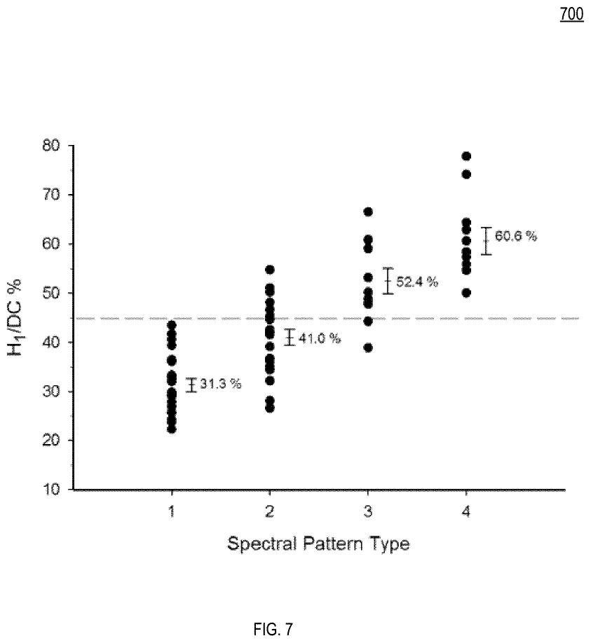

With the aim of developing an objective method to classify spectral patterns according to their degree of organization, numerical values from 1 to 4 are assigned to the spectral ensembles shown in FIG. 6. These values range from poorly organized (Pattern 1) to highly organized (Pattern 4). Eighteen independent observers were instructed to classify each two-hour spectral ensemble from the present study (n=66) according to the patterns shown in FIG. 6. The mode of the 18 values assigned to each ensemble by the independent observers were calculated. The average H1/DC from the 48 spectra for each two-hour ensemble was also calculated. FIG. 7 shows the mode of the observers' classification plotted as a function of the average H1/DC ratio.

All spectral ensembles classified as pattern 1 had H1/DC<45%, whereas all those classified as pattern 4 had H1/DC 45%. Also shown in FIG. 7 are the mean±95% confidence limits for H1/DC corresponding to each spectral pattern type. There is clear relationship between spectral pattern type and H1/DC, with each type pattern separated by an increment in H1/DC of approximately 10%. When combining patterns 1 and 2 into a “disorganized” group and patterns 3 and 4 into an “organized” group, a H1/DC of approximately 45% or greater differentiated them with a sensitivity of 84.1% and a specificity of 91.3%. The close relationship between the spectral pattern and H1/DC allows for display of H1/DC as a measure of asynchrony at 2.5 minute intervals.

The above shows that obtaining the frequency spectra provides a useful indication of patient-ventilator asynchrony. Display of H1/DC allows for careful adjustment of a ventilator to correct and eliminate or reduce patient-ventilator asynchrony. Obtaining H1/DC provides a useful indicator of detecting patient-ventilator asynchrony. Display of the H1/DC also allows careful adjustment of a ventilator to correct and eliminate or reduce patient-ventilator asynchrony. Moreover, outputting a signal corresponding to H1/DC by the CPU 60 to the ventilator 20 allows automatic adjustment of the ventilator to correct for patient-ventilator asynchrony based on the calculated H1/DC in accordance with the present invention.

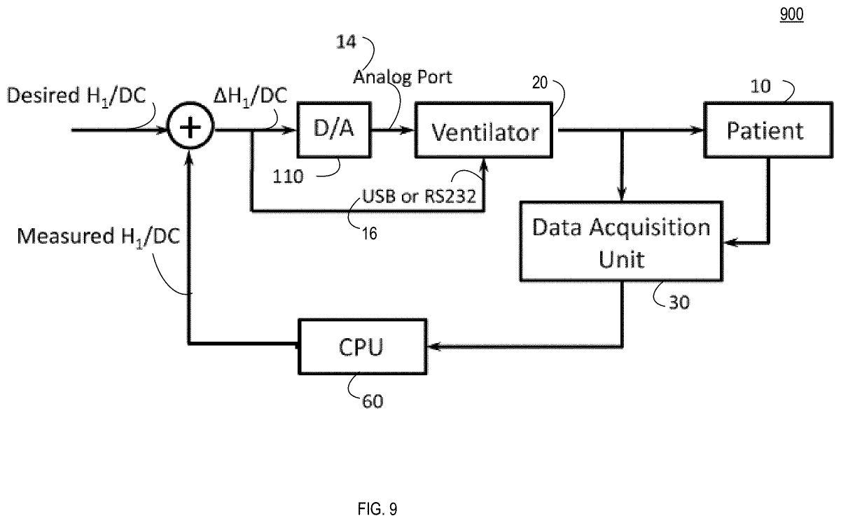

The measure of spectral organization, such H1/DC or the others mentioned above, or information derived therefrom, can alert for the presence of asynchrony to physicians and nurses caring for the patient so that therapeutic measures may be taken. Measures can include, for example, manual changes in ventilator settings, such as the respiratory rate and ventilation volumes. Other therapy could be greater sedation or the use of paralytic agents. It also possible to utilize the information provided by H1/DC to make ventilator changes automatically, through the use of a feedback system aimed at maintaining the level of patient-ventilator asynchrony within a desired range. As will be recognized to those skilled in basic feedback control, the CPU 60 could be programmed to compute the difference (ΔH1/DC) between the measured H1/DC and a previously set point (desired) for H1/DC and use difference (ΔH1/DC) as a feedback signal to make changes on the mechanical ventilator 20 such as schematically illustrated in the exemplary feedback system shown in FIG. 9.

In an exemplary feedback system such as shown in FIG. 9, the feedback signal (e.g., ΔH1/DC) is applied to the ventilator 20. As shown, the feedback signal can be applied either as an analog voltage to an analog port 14 using a digital-to-analog converter 110 or as a digital signal delivered to an input port 16 such as a USB port or a RS232 port of the ventilator 20. For example, if decreases in H1/DC below a set value, for example 40% are determined by the CPU 60, the CPU 60 can send a signal to the ventilator 20 to change inspiratory volume delivered or in the number of breaths. The ventilator 20 also could be programmed to switch from one mode of ventilation, for example, pressure controlled mode to another, such as volume controlled mode.

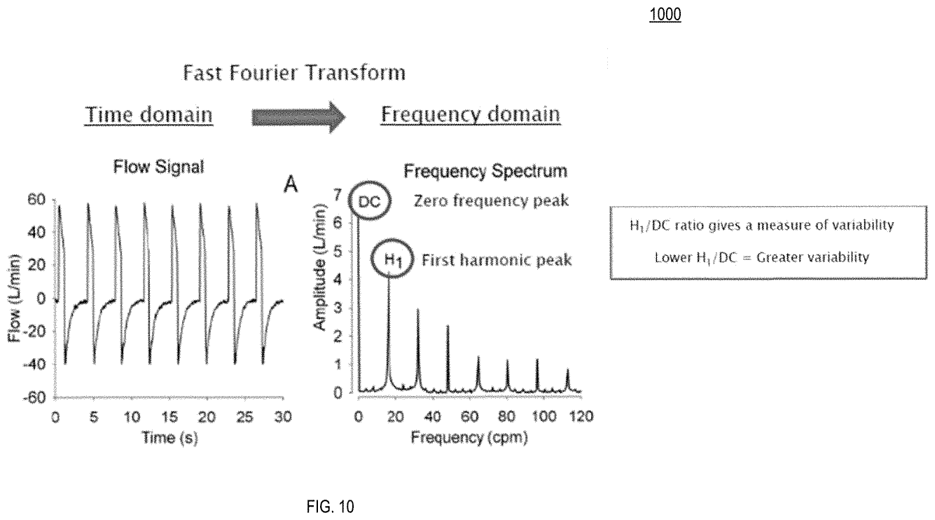

FIG. 10 depicts an exemplary embodiment of a diagram 1000 illustrating a fast Fourier transform between the time domain and frequency domain and the zero frequency peak DC voltage level as well as the H1 first harmonic peak, the ratio of which is illustrated, according to an exemplary embodiment.

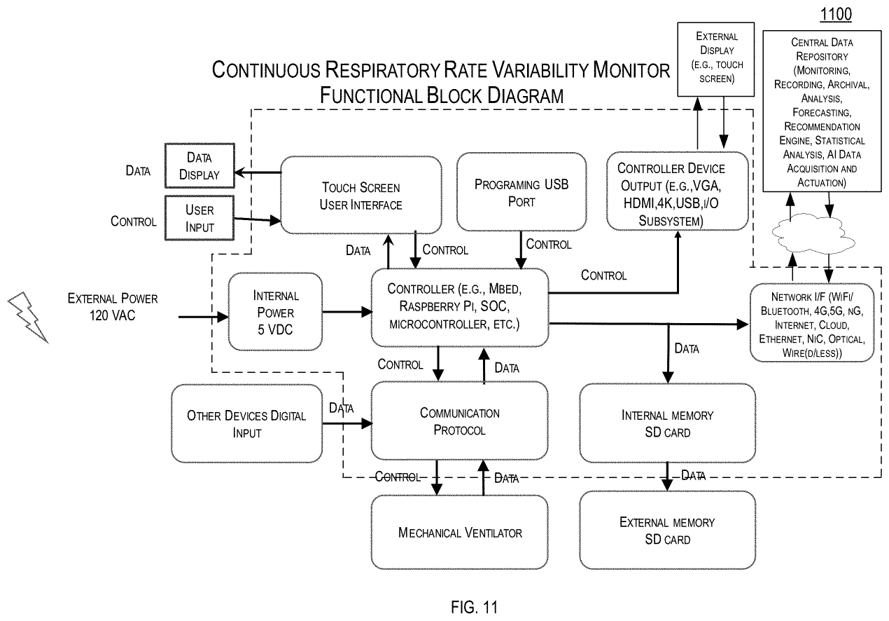

FIG. 11 depicts an exemplary embodiment of a diagram 1100 illustrating a top level functional block diagram of an example continuous respiration monitor, according to an exemplary embodiment of the present invention.

Diagram 1100 includes an exemplary functional block diagram including exemplary interfaces, including, e.g., but not limited to, exemplary controller, such as, e.g., but not limited to, an mbed processor, or alternatively a raspberry pi, or other system on a chip (SOC), or other microcontroller, to control, and/or provide exemplary interfaces such as, e.g., but not limited to, video interface (e.g., HDMI, VGA, SVGA, XGA, etc.), I/O interface (e.g., USB, etc.), and/or power interface (e.g., USB, coax plug, etc.), wired I/F (e.g., ethernet, NIC, RJ45, optical, coax, etc.), and/or wireless (e.g., WiFi, BLUETOOTH, 4G, 5G, nG, WiMax, etc.), and exemplary external memory (e.g., external SD card, USB memory stick, etc.), ventilator interface (e.g., interface to mechanical ventilator, alternative interface to intelligent ventilator, other interfaces, etc.), potential connection to a local, and/or remote central repository for exemplary processing including, e.g., but not limited to, monitoring, recording, archival, analysis, forecasting, a recommendation engine, statistical analysis, AI data acquisition and/or actuation, storage, backup, retention, interface to electronic medical records (EMR), etc., according to an exemplary embodiment. According to one exemplary embodiment, a mechanical ventilator can be tightly coupled to a monitor, or integrated as an intelligent mechanical ventilator. In another embodiment, the monitor can be separate as shown and can serve to interface bidirectionally with the mechanical ventilator. According to one exemplary embodiment, other interfaces can include interface to a pulse oximeter, such as a ZacVrate or Mosimo pulse oximeter, or other O₂ interface can be coupled to an external interface, according to an exemplary embodiment. According to one exemplary embodiment, a capnograph or other CO₂ meter can be coupled to an external interface. According to one exemplary embodiment, a blood pressure monitor, and/or a pulse meter, etc., can be coupled to an external interface, according to an exemplary embodiment. According to one exemplary embodiment, data can be captured from monitoring of the mechanical ventilator, and can include intelligent monitoring and recording and actuation and actin, including, e.g., but not limited to, acquiring information (e.g., pressure and volume curves), keep a record of what was done, provide a way to actuate, i.e., e.g., to adjust the ventilator to give more volume, increase rate, adjust pressure, etc. while adjusting ventilation, keeping a record of what was done, and maintaining and keeping the Clinician informed, maintain a library, perform continuous learning, provide for output to an EMR record, can be used by others, allowing updating via feedback to the AI library, of results of actions, e.g., success or failure, etc., informing a clinician that a change will be made, allowing making small, incremental changes, e.g., change by 1 breath/minute, etc., and the information and changes can be continually sent up, continuously gathering data and forwarding from the ventilator and/or monitor up to the central data repository, for monitoring, recording, storage, analysis, predictive forecasting, recommendations, and based on many instances and separate outcomes, as well as the interventions, and results, whether, e.g., good, neutral, or deleterious outcome, etc., and/or can use feedback and continuous iterative improvement, to improve over time, using machine learning to identify recommended future changes, according to an exemplary embodiment.

### Exemplary Device Components

The Respivar System can include, according to an exemplary embodiment, the following example components:
User interface—For entering patient information, displaying patient data, and indicating alarms.
Data Collection Ports (See back panel)—These consist of three male DB9 connections labeled “Input 1”, “Input 2”, and “Ventilator”. They are used for interfacing to the ventilator, pulse oximeter, and other monitoring devices.
A female output DB9 port: This is reserved for future use.
Power Switch (On/Off)—For controlling power to the device.
Storage (SD Card, Export)—For recording and retrieving data from system to view on other devices.
Version Control (Network, I/O)—For updating software.
Network Port—This is reserved for future use.

In addition, there can include a clamp to attach the monitor to an I.V. pole or directly to the ventilator, according to an exemplary embodiment.

### Intended Use

The Respivar System is intended to aid in the decision making process for sedative administration, according to an exemplary embodiment. It can monitor the degree of synchronous breathing between patient and ventilator, according to an exemplary embodiment.

### Intended User

The Respivar System, according to an exemplary embodiment, should be used only by those who:
Are professional health care providers, and
Have received training in the use of this system, and
Have experience in the use of mechanical ventilation.

### Intended Use Environment

The Respivar System, according to an exemplary embodiment, should be used only in facilities whose primary purpose is to provide healthcare to mechanically ventilated patients.

### Exemplary Operation Overview

### Exemplary Workflow Summary

The following summary procedure, according to an exemplary embodiment, provides an overview of the exemplary operation of the Respivar System.

### Set Up:

1.) Use clamp system to mount the monitor either to a nearby infusion pole or directly to the ventilator. Device should be firmly mounted.

2.) Plug male end of male-female DB9 cable into ventilator and tighten screws. Plug female end of same DB9 cable to “Ventilator” port on the back of the device and tighten screws.

3.) Plug power cord into a standard 120V wall outlet.

4.) Ensure there is an SD Card in the “SD Card” slot on the monitor's back panel.

5.) Flip the On/Off rocker switch to the “On” position. The device should turn on and a keypad is displayed on the screen. re there is an SD Card in the “SD Card” slot on the monitor's back panel.

### Location of RS-232 Port on Ventilator

Servo-i Ventilators have an RS-232 port located on their side in their module compartment depicted below [see (9)]. Servo-s Ventilators have an RS-232 port located on the back.

### Device Use:

1.) On turning on the device, it will perform a series of system tests to ensure proper functioning. These tests will last for 5 seconds. During this time a black screen with green text, “Initializing . . . ”, in the upper left corner will be displayed.

In the event of a failed test, the screen will display an error message as well as instructions for resolving the issue. For example, if there is no SD card present on turning on the device, the screen will display the message “External SD Card inoperable. Please reinsert”. The device will then wait until an SD card is inserted before resuming normal operation.

2.) After the device finishes its system tests, the intervention arm select screen will be displayed. A prompt, “Open envelope and press designated button.”, and two buttons “Study”, and “Control” are displayed. Open the provided envelope and press the indicated button.

3.) After pressing either the “Study” or the “Control” button, a 10-digit keypad is shown. Alongside are three buttons labeled “Delete”, “Accept”, and “Back”. Use the keypad to enter the patient's ID number and then press “Accept” to confirm. The study number must be 10 digits or it will not be accepted. If at any time one wishes to delete the last digit entered, press “Delete”. If one wishes to return to the Intervention Arm Select screen, press “Back”.

4.) After pressing the Study Number Keypad “Accept” button, the Current Time

Keypad screen will show. Use the keypad to enter the current time in the following 10 digit format:: 2-digit year (Y), 2-digit month (M), 2-digit day (D), 2-digit hour (24-hour clock) (H), 2-digit patient minute (M). and then press “Accept” to confirm.

5.) After pressing the Current Time Keypad “Accept” button, the Registered Time Verify screen will show. On this screen will be two two text boxes and two buttons: “Back”, and “Correct”. The uppermost text box will display either the word “Valid”, or the word “Invalid”. If it displays “Invalid” then the current time that was registered on the previous screen contained an error. For Example, the date registered may have been February 30th.

The lower text box will display the date in standard United States date and time notation (Month-Day-Year and 12-hour clock w/ AM/PM suffix).

Verify that this time is correct and press either “Back” to return to the Current Time Keypad screen to retry or “Correct” to continue.

6.) After pressing the “Correct” button, one of four screens will show: Cable Disconnected Screen: Will appear if the device is not properly connected. Check the connections of the RS232 cable between the ventilator and the Respivar. Once properly connected, either the Standby Screen, Gauge Screen, or Control Screen will display.

Standby Screen: Will appear if the device is properly connected and the ventilator is on standby mode.

Blank (Control Arm) Screen: Will appear if the device is properly connected, the ventilator is active, and the “Control” button was pressed on the Intervention Arm Select screen. The monitor will record data from the ventilator until disconnected but will not display results.

Gauge (Study Arm) Screen: Will appear if the device is properly connected, the ventilator is active, and the “Study” button was pressed on the Intervention Arm Select screen.

The gauge displays three colored arcs and a pointing arrow. In addition, there is a button, “Sedation Decreased” used to indicate action was taken, and a rectangular flashing alarm region. The graduated gauge displays the degree of respiratory variability. When the arrow points to red, it indicates very low variability, usually seen in patients who are overly sedated and synchronous with the ventilator. Green corresponds to synchrony and yellow to asynchrony

The gauge will initially display the leftmost value (25) until the first update. The first update occurs within 2.5-5 minutes. The gauge will then update every 2.5 minutes. Its value represents the 5 most recent minutes of data.

There will also be a red flashing alarm if the H1DC displayed by the gauge is in the red zone. The alarm is only visual and does not make sound.

7.) After pressing the “Sedation Decreased” button, the countdown screen will show. A clock shown on the screen will begin a 30-minute countdown. The user cannot navigate away from this screen. After the countdown finishes the gauge screen will be shown. While on this screen data continues to be collected and H1DC calculated.

### Patient ID Format

Patients should be submitted to the Respivar via keypad in the following format:

10 digits total: 4-digit year (Y), 2-digit month (M), 2-digit day (D), 1-digit hospital ID (H), 1 digit patient ID (P). YYYYMMDDHP

Hospital ID's are assigned. Patient ID's should be entered starting at 1 for the first patient each day and enumerating from there

EX) Patient 1, Hospital 9: December 1st, 2017: 2017120191.

### Disconnecting the System

To safely disconnect and stop data collection:

1) Physically disconnect the Respivar System from the ventilator.

2) Wait 5 seconds, Turn the Respivar off using the rear On/Off switch.

Note: The battery module will recharge as long as the Respivar is connected to AC power. It is not necessary to leave the Respivar turned on.

### Power Supply

Introduction

The Respivar System is equipped with one 7800 mAH AC to USB power supply which can maintain battery operation for up to 15 hours. The Respivar will automatically operate properly using 100-120 Volt AC outlets.

The Respivar's battery module will automatically supply 5-volt DC power in the case of an AC failure, ensuring that the Respivar settings and stored data remain intact.

AC power Failure

In the event of an AC power failure or disconnection, the Respivar switches to battery operation. In the event of complete loss of power, the device will shut down and all settings will be saved until the device is powered again.

### Transport and Storage

### Transport

Transporting Respivar with ventilator, follow facility guidelines and:

Leave Respivar turned on.

Be sure clamp is securely attached and locked

Be sure all cables are securely attached.

Be sure the batteries are fully charged.

Inspect the Respivar for damage.

### Storage

Turn off but, keep the Respivar plugged in so that the battery maintains a full charge.

Be sure the system is not exposed to temperatures below −25 C or above +60 C

Be sure the system is not exposed to a relative humidity above 95 percent.

Microcontroller (Nucleo F767ZI)

https://os.mbed.com/platform/ST-Nucleo-F767ZI/

This microcontroller was chosen because it was one of very few with the necessary number of UARTs required by the project. Below is a list of features it's features. Prominently, we are currently using 6 of the 8 UARTS and 2 of the 6 SPI channels.

### Microcontroller Features

STM32F767ZIT6 in LQFP144 package

ARM®32-bit Cortex®-M7+DPFPU+Chrom-ART™ Accelerator

216 MHz max CPU frequency

VDD from 1.7 V to 3.6 V

2 MB Flash

512 KB SRAM

GPIOs (114) with external interrupt capability

12-bit ADCs with 24 channels (3)

12-bit DAC channels (2)

USART/UART (8)

I2C (4)

SPI (6)

General Purpose Timers (10)

Advanced-control Timers (2)

Basic Timers (2)

Low-power Timers (1)

Watchdog Timers (2)

CAN 2.0 B active (3)

SAI (2)

SPDIFRX 4 inputs

SDMMC

Camera Interface

LCD-TFT

USB 2.0 OTG HS/FS

Random Number Generator (TRNG for HW entropy)

Ethernet

### RS-232 Connections

Each box has four DB9 connections, three male and one female. Each DB9 connection uses three pins: pin 5 to ground, pin 3 to transmit data, and pin 2 to receive data. Begin by soldering one long wire to each of the three pins for all four of the db9 connections.

Mount the DB9 connections to the back panel. Apply superglue to the nuts before tightening them. This is to ensure the nuts do not come loose from daily wear as people plug and unplug RS-232 connections from the monitors. Add heat shrinks to exposed wire.

### Switch

Cut a USB to USB cable in half. Strip the insulation and wires. Add heat shrinks to the wires. Solder the red and black wires of the USB cable to two separate tabs of the bottom pole of the switch. Solder two separate wires to the corresponding tabs of the middle pole of the switch. Add heat shrinks to the wires. Attach to the panel. Tighten by gripping the red body of the switch and the outer rubber boot. Twist both counter clockwise. Hold the rubber boot firm and twist the red body clockwise. Repeat until tight.

### Push Button

Solder two wires to the two tabs of the push button and add heatsinks. Attach the push button to the panel using the accompanying nut.

### USB-B to Micro USB

Attach to panel with accompanying screw and nut (comes with purchase in plastic bag).

### SD Card Extension

Secure the panel tightly in place, either in the box as pictured below or with helping hands. Epoxy may leak and be hard to control. Remove the Rj45 connection before continuing to make sure you do not accidentally glue it in place as well. Place the SD Extender in the hole in correct orientation (the small black square on the extender should be facing up). The extender will fit loosely in this hole. Secure the extender in place with a helping hand before continuing. Wear PPE according to recommendations on epoxy. Mix epoxy according to instructions on canister and apply generously around the extender. Ensure epoxy completely fills in the gaps and does not leak as it dries.

### Panel Mount RJ45 Extension

Attach to panel with 4-40 screws. Optional: Use file on bottom edge of cutout to allow extension to fit snugly.

### Battery

Use scissors to cut two velcro strips to the length of the battery. Place the two hook strips (pointy) in the location indicated below. Place the two loop strips (fuzzy) on the edge of the battery that will be resting against the back panel. See below image for orientation of battery.

### AC Adapter

Fit a strain relief boot into the hole on the bottom right of the back panel as pictured below. The fit will be tight. Use tools (pliers or other pointed objects) to help wedge in the boot if needed. The DC jack of the AC Adapter can not fit through the boot. Cut the wire to the desired length (suggest at least 6 inches from the DC jack).

Leave enough room to tie a knot on the inside of the box with the wire, to splice the wire back together and cover in heatshrink, and to account for a mistake while stripping the wires (the wires are thin and easy to accidentally fully cut rather than strip).

Run the cable through the boot. Strip the insulation from the cables, both the DC jack cable and AC Adapter side of cable. There will be two wires, one black and one red. Be sure to strip the insulation far enough to be able to fit a heat shrink on the exposed wires. Put one heat shrink on each wire and a larger heat shrink on the cable itself. Splice the black wire to the black wire and red to red. Fit the heat shrinks and tie a simple knot in the wire to keep it from being pulled out of the box. Tighten the knot as much as possible.

### Printed Circuit Board (PCB)

The PCB is designed to fit all the peripherals nicely to the microcontroller. This includes the two SD cards, the two max232's for the four RS232 connections, the serial screen connection, and the pushbutton. It does not have a pin for the power (E5V) and ground (any GND) from the AC adapter. Those must be soldered directly to the microcontroller. A further limitation is the PCB was designed such that input 2 uses USART 3. USART 3 is shared with the virtual com port used for reprogramming the monitor. Thus, we can not both use input 2 and reprogram the monitor over USART 3. We opt to not connect input 2. To fix this, future PCB's should reroute the pins to a different USART.

Break off enough male header pins of the appropriate lengths to solder to connectors CN7, CN8, CN9, and CN10. Keep in mind it is easier if the male header pins are kept connected to each other rather than breaking them off into individual pieces and soldering.

For each connected series of male header pins, place them into an appropriate slot on the PCB and first solder only a single header the the PCB. The pins will likely be misaligned at this point. Reheat the pin connected to the PCB and use your fingers to adjust the series of connected pins until they are straight. Verify that it is straight by fitting the PCB into the microcontroller. Do this for all connectors. Once all connectors are straight and fit into the microcontroller. Solder all the male header pins to the PCB.

Solder the two micro SD breakout boards to the PCB. the SD cards are labeled and so is the board. Fit the header pins into the appropriate labels. Solder a single header pin first. Then reheat the pin and adjust the micro SD breakout board until it is level and not touching the PCB. solder the remaining pins. Repeat for the second micro SD breakout board.

Solder a 5 pin long male header to the top left of the PCB board. One pin first, then straighten, then the rest of the pins as usual. Pay attention to the orientation of the pins. The longer side is on top this time. This will be used for serial communication between the microcontroller to the screen.

Solder the capacitors for the MAX232's to the PCB. Ensure the polarity is correct before soldering. Each MAX232 will have four capacitors. Use a wire cutter to snip the capacitor's wires close to the PCB.

Solder an integrated chip holder into the designated spot on the PCB.

Repeat for the upper MAX232 and check that the board fits in the microcontroller and that no wires from the PCB touch the microcontroller.

Finally solder terminal blocks to the bottom row of of the PCB labeled RX, TX, GND, etc.

### Assembling Enclosure

At this point all the wiring for the back panel and soldering for the PCB should be finished and all the remains is connect the back panel to the PCB, microcontroller and screen.You should have a box that looks like the picture below. It is easiest if you have also epoxied the SD extender at this point.

Remove the battery if it is in place and trim the wires to a more appropriate length. The easiest way to find the correct length is to place the PCB in the microcontroller. Then place the microcontroller into the the two small brackets on the bottom of the box as this is where the microcontroller will eventually rest. Drape the wires from each component to the intended spot past the microcontroller then snip the wire leaving a small amount of excess wire.

After trimming the wires it easiest to then separate them according to where they will be attached to the PCB. Tape the pushbutton into one unit, the RX and TX of ‘output’ into one unit, the RX and TX of ‘input ‘1 into one unit, and all the wires of ‘ventilator’, all the wires of ‘input 2’ (if input 2 was fixed, otherwise just cut the wires of input 2 off and ignore), the GND of ‘input1’, and the GND of ‘output’.

Begin by soldering the switch to the microcontroller and the pushbutton to the PCB. Power of the switch should be connected to E5V on the microcontroller and ground of the switch to any ground on the microcontroller. The push button should be soldered to the PCB to the pins labeled ‘PF2’ and ‘GND’ as indicated. Orientation does not matter for the pushbutton.

Based on the two pictures below, solder ‘output’ to the upper set of pins labeled ‘TX’ and ‘RX’ on the PCB. Solder pin 3 of the RS232 connections to ‘TX’ on the PCB, and pin 2 to ‘RX’ on the PCB. Repeat for ‘input 1’ to the lower set of pins labeled ‘TX’ and ‘RX’. The ground for these two RS232's will be screwed into the terminal blocks.

Attach the PCB to the microcontroller. Ensure jumper 3 is set to ESV. It is set to U5V by default. U5V is for 5V usb power. E5V is for the external power pin that we just soldered the switch to. Screw the wires from ‘ventilator’ to the terminal blocks. Be sure to screw pin 3 to ‘TX’ and pin 2 to ‘RX’ as labeled. Screw the grounds of ‘ventilator’, ‘input 1’, and ‘input 2’ into any of the terminals labeled GND. Cut the wires of ‘input 2’ away unless PCB was redesigned.

Attach the RJ45 extender and microusb to the microcontroller. Make sure the wire coils in a way that does not push hard on the microcontroller. Attach the DC jack and USB cable to the battery. Press the black switch on the battery. The red light and green light should both be turned on if the battery is plugged into the wall. This indicates the battery is in the correct continuous power mode. The red light should be on even if the battery is unplugged from the wall.

Screw in the base of the mount to the bottom of the box. Super glue the micro USB and the RJ45 extender to the PCB columns indicated by the red circles.

Program the screen and attach it to the microcontroller using the jumper cable that comes with each screen. Insert the SD extender into the external micro SD breakout board. Insert a microSD card into the internal SD breakout board. Put the cover on. The hardware of the box is now complete.

Downloading MBED Software

Note: When using E5V as an external power supply, it is mandatory to power the board first using E5V, then to connect the USB cable to the PC. In this way enumeration succeeds thanks to the external power source.

Note: If this order is not respected the board may be powered by USB first, then by E5V. Risks include damaging the PC and enumeration failure. The board will not be powered correctly.

Note: Before connecting the Nucleo-144 board to a Windows 7, 8, or XP PC via USB, a driver for ST-LINK/V2-1 must be installed. It can be downloaded from th www.st.com website.

1.) Log in to the mbed compiler web site (https://developer.mbed.org/), and set up the necessary drivers (see note) to compile successfully.

2.) Select “Compiler” from the top menu. A new window will open.

3.) Navigate to the most recent version of code. Click Compile. Downloaded code will appear at the bottom left of most browsers. Ensure downloaded code is on mbed os version 136 and no errors appear under “Compile output for program: xxx”.

4.) Power on the external power supply (battery/AC Outlet). The green LED LD6 should be turned on. 5.) Wait a few seconds and connect the PC to the USB connector CN1 (USB-B Connector on back of box). 6.) Drag/save the downloaded code of the device into the mbed folder.

7.) Code will run immediately. You may disconnect the USB at any time and return device to operation.

Touch Screen Software

Be sure to have the most up to date PMMC on the screen.

Please review first project application note from 4dsystems: 4D-AN-00106

https://www.4dsystems.com.au/appnote/4D-AN-00106

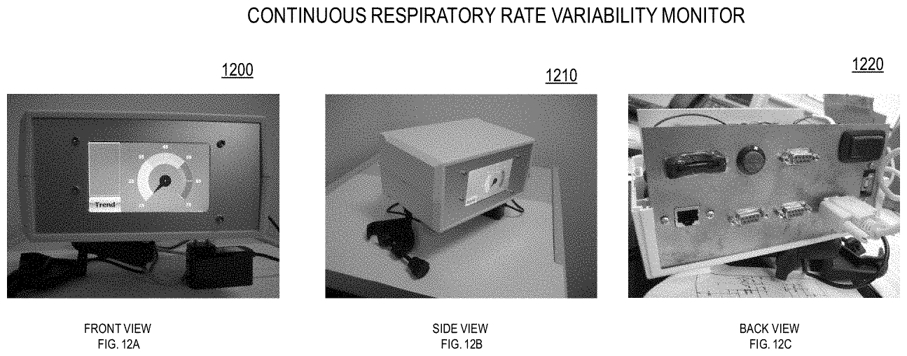

FIGS. 12A, 12B, and 12C depict exemplary embodiments of diagrams 1200, 1210, and 1220, respectively, illustrating an exemplary front view and exemplary back view of an example box and stand setup of the example continuous respiration monitor including a photograph image of the front of the box powered on mounted in a stand, and a photograph image of the back of the box mounted in an example stand, respectively, according to an exemplary embodiment of the present invention.

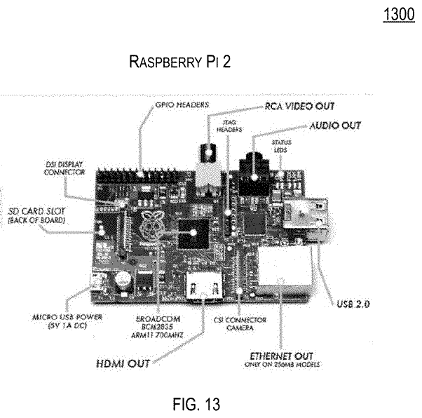

FIG. 13 depicts an exemplary embodiment of a diagram 1300 illustrating a raspberry pi 2 micro controller as could be used in one exemplary embodiment of monitor 1100, according to an exemplary embodiment of the present invention.

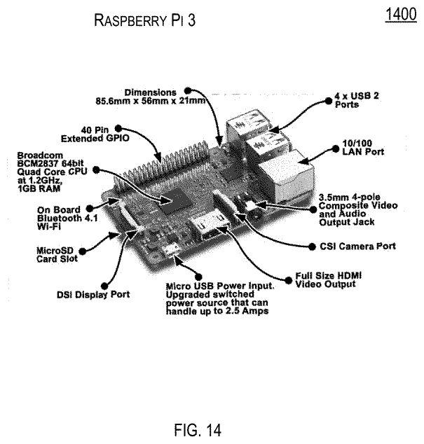

FIG. 14 depicts an exemplary embodiment of a diagram 1400 illustrating an an exemplary embodiment of a raspberry pi 3 micro controller as could be used in one exemplary embodiment of monitor, according to an exemplary embodiment.

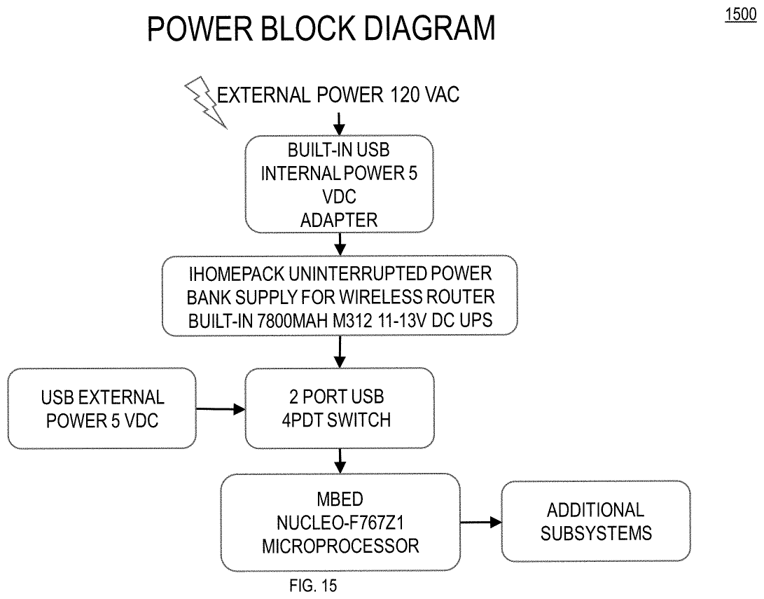

FIG. 15 depicts an exemplary embodiment of a diagram 1500 illustrating an example power overview of the example continuous respiration monitor device including an example power block diagram, the example MBed Nucleo microcontroller, powers all other example subsystems, red/blue illustrated arrows depict combined communication flows representing USB connectors, such as, e.g., but not limited to, an example USB-B micro to USB-B micro connector with panel mount between wall outlet to Apple Wall Adapter, an example USB-B to USB-B micro connectors between Apple Wall Adapter and 3000 mAh example battery with pass through charging, according to an exemplary embodiment, an example USB-B to USB-B micro connector between the 3000 mAh battery and USB-B micro break out board, example coupling of the USB-B micro break out board to example serial included switch, and to USB-B Micro break out board, and example USB-B micro to USB-B micro connector coupling to Mbed, according to an exemplary embodiment of the present invention.

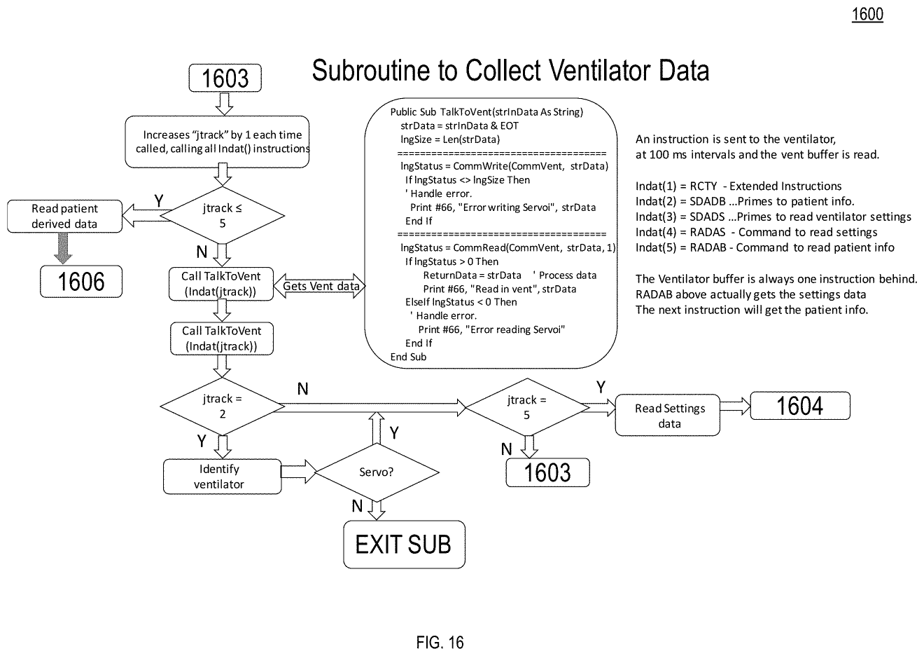

FIG. 16 depicts an exemplary embodiment of a diagram 1600 illustrating example an exemplary routine to collect example ventilator data, including example process steps 1603, decision processing and performance of illustrated exemplary steps 1606, and 1604 to collect ventilator data, sending exemplary messages at exemplary intervals, and processing decisions, sending instructions, and reading ventilator buffer, settings, and patient information, according to an exemplary embodiment of the present invention.

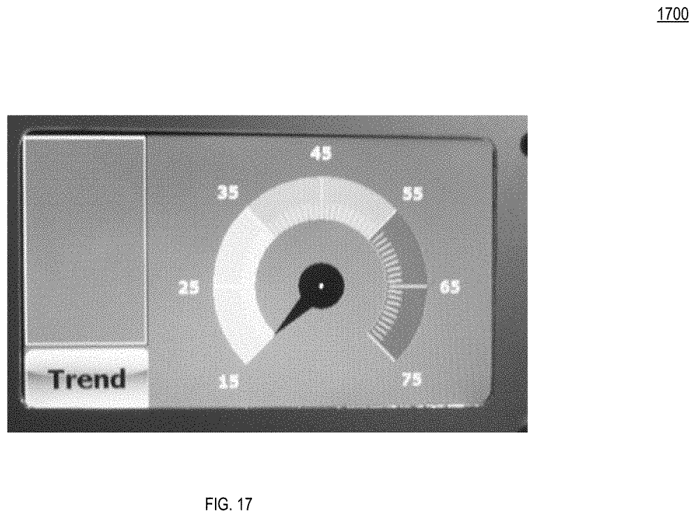

FIG. 17 depicts an exemplary embodiment of a diagram 1700 illustrating example output display with yellow, green, and red sectors, clockwise from left to right, according to an exemplary embodiment of the present invention.

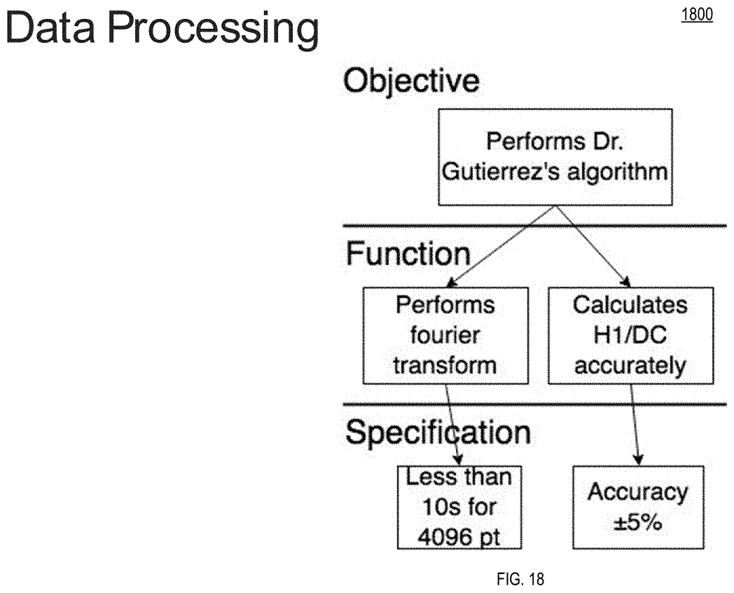

FIG. 18 depicts an exemplary embodiment of a diagram 1000 illustrating example data processing functions of the example continuous respiration monitor device, according to an exemplary embodiment of the present invention.

### Machine Learning Algorithms to Monitor Mechanical Ventilation

Rationale: Mechanical ventilation requires frequent assessment of patient-ventilator interaction. It is desirable, therefore, to have an intelligent monitor perform this function continuously and automatically.

Objective: Develop machine-learning algorithms to evaluate patient-ventilator interaction.

Methods: We enrolled 44 patients on invasive mechanical ventilation. Ventilator data and airway signals were captured continuously and displayed as 2.5-minute epochs for classification by clinicians experienced in mechanical ventilation and asynchrony identification. These data, along with the frequency spectra of airway signals, formed a database used to train Random Forest classifier algorithms to: 1) recognize ventilator disconnection; 2) detect asynchronous breathing; 3) classify asynchrony type; 4) grade its severity; and 5) identify breath-stacking. 70% of the database comprised a training set and 30% a test set. Algorithm accuracy was judged by the percentage of test set's epochs predicted correctly. Reliability was assessed from the measured agreement between clinicians and algorithms when classifying identical data sets.

Measurements and Main Results: We classified manually over 50,000 epochs, encompassing approximately 2.6 million breaths. Ventilator disconnection and asynchronous breathing were detected with 100% and 91% accuracy, respectively. Accuracy for asynchrony classification, severity grading and breath-stacking identification was 82%, 87% and 93%, respectively. There was substantial agreement between expert clinicians and the algorithms. Reliability was related to expertise in asynchrony classification (p<0.05). 28-day mortality was associated with greater time being asynchronous (p=0.015).

Conclusions: It is feasible to develop accurate machine-learning algorithms to monitor patient-ventilator interaction. Algorithm reliability, however, depends on the classifiers' expertise. Asynchrony classification standards are needed to promote algorithm development.

### Methods

This was a prospective, sequential, observational study performed at The George

Washington University Hospital ICU (IRB # 101228). Informed consent was obtained from all patients or surrogates. We enrolled adult patients of any gender within 24 hours from the initiation of invasive mechanical ventilation. Patients were ventilated with Servo ventilators (Getinge, Solna, Sweden) and monitored for four days, or earlier if weaned from the ventilator. We excluded, or removed from the study, patients requiring hourly neurological examinations or those treated with paralytic agents. De-identified demographics, diagnoses and hourly use of i.v. propofol were later extracted from the clinical chart. We also noted days on mechanical ventilation, ICU and hospital length of stay, and all-cause mortality at 28 days.

Clinicians caring for the patients chose the manner of ventilation with no input from the research team. We acquired data continuously with a CS1 monitor (Respivar LLC, Fairfield, Pa.) connected to the ventilator's RS-232 port. This monitor directs the ventilator to sample airway signals, computes their frequency spectra and stores the data as consecutive 2.5-minute time-windows. The monitor also gathers ventilator settings and ventilator measured breathing data, such as the respiratory rate and expired tidal volume.

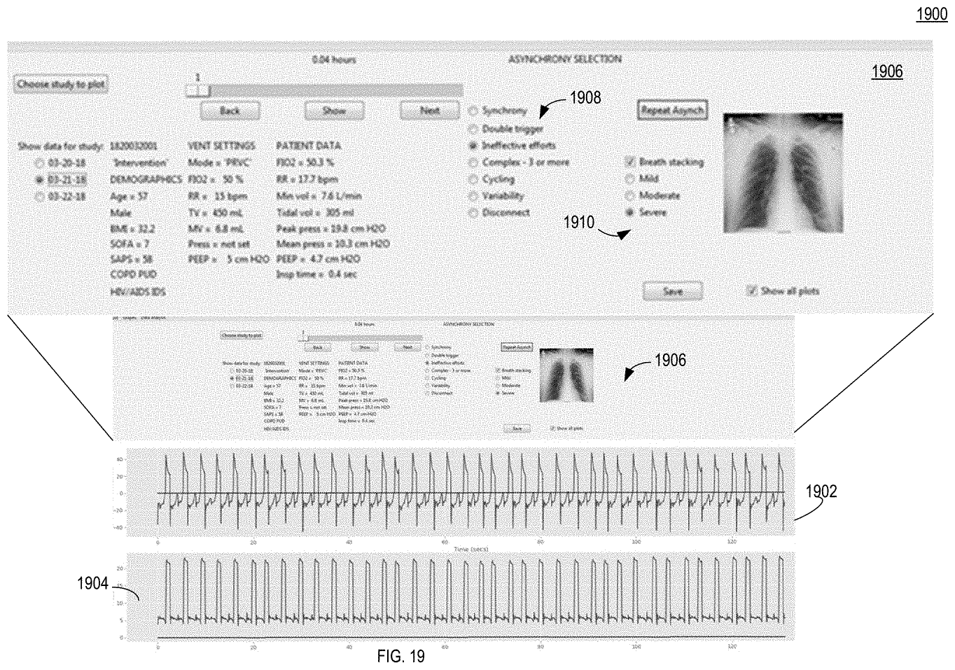

Monitor data were transferred to a digital computer for analysis once the patient completed the study. Software was written to display the data from each time-window, or epoch, as a composite dashboard (FIG. 19) showing the time and date when collected, demographics, ventilator settings, measured breathing data and a chest roentgenogram taken on the day of enrollment. Airway flow and pressure signals sampled at 31.25 Hz for 131 seconds were displayed in a separate panel. Radio buttons listed choices for breathing pattern classification, severity, and breath-stacking (5).

FIG. 19 depicts an exemplary embodiment of diagram 1900 illustrating exemplary dashboard 1906, including an exemplary blown up/zoomed in version illustrating exemplary clinician interface to gather exemplary monitoring data and clinician scoring, by an example clinician based on interpreting of ventilator data streams 1902, 1904, and captured scoring 1908, 1910, for a given epoc, according to an exemplary embodiment of the present invention. Screen shot of an epoch's dashboard showing the flow and pressure signals sampled at 31.25 Hz for 131 seconds. Ineffective efforts are seen within each breath cycle. Also shown are demographics data, ventilator settings and ventilator measured patient data. The left radio buttons and the slider allow access to data from different days and times. Other radio buttons are used for epoch classification. A chest roentgenogram taken on the day of enrollment is shown as a small picture capable of enlargement when clicked.

FIG. 19. depicts an exemplary embodiment of an exemplary composite dashboard according to an exemplary embodiment of the present invention, including an exemplary Screen shot image of an epoch's dashboard showing the flow and pressure signals sampled at 31.25 Hz for 131 seconds; exemplary Ineffective efforts are seen within each breath cycle; Also shown are exemplary demographics data, exemplary ventilator settings and exemplary ventilator measured patient data; the exemplary left radio buttons and the exemplary slider graphical user interface element can allow access to data from different days and times, according to an exemplary embodiment. Other radio buttons, or other user interface elements can be used for epoch classification, according to an exemplary embodiment. An exemplary chest roentgenogram taken on the day of enrollment is shown as a small picture capable of enlargement when clicked, according to an exemplary embodiment.

Epoch scoring: Five clinicians with expertise in mechanical ventilation and asynchrony identification (assessors) scored each epoch by visual inspection. Assessors were instructed to classify epochs based on the type of asynchronies observed in the airway signal panel according to accepted definitions of double triggering, ineffective effort, and either delayed or premature cycling (6). We defined asynchronous epochs as those displaying four or more asynchronous breath-cycles. Whenever two types of asynchronies were seen within the signal panel, the epoch was classified according to the most frequently observed asynchrony type. Epochs displaying all three types of asynchronies were classified as “complex”. Some epochs were classified as “ventilator disconnect” when presenting a pattern characteristic of this condition. Epochs with <4 asynchronies were classified as either “synchronous” or “variable breathing”, depending on their degree of breathing regularity. (See the online data supplement for examples of asynchrony identification)

All epochs were graded as mild, moderate and severe according to the assessor's perceived degree of airway signal disruption. Synchronous epochs were automatically graded as mild. Epochs where the end-expiratory flow failed to reach the zero baseline in >4 breaths were further classified as breath-stacking. Due to the large number of epochs produced during the study, each epoch was classified by only one assessor. All information pertaining to an epoch was saved as a row, or instance, in the database.

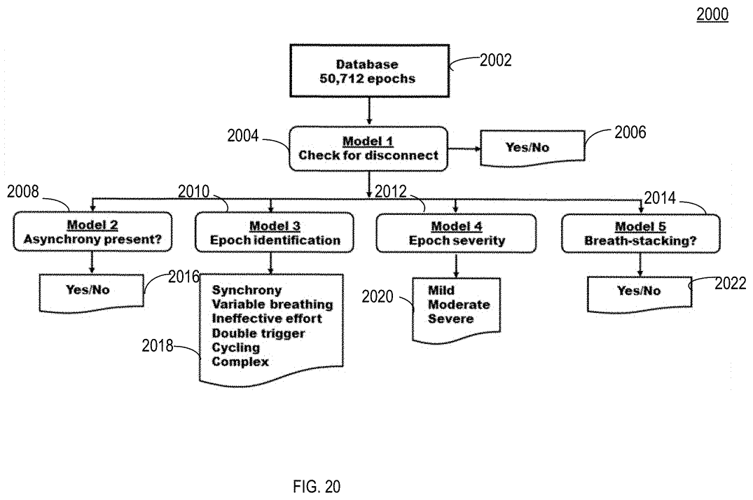

Machine learning algorithms: We used 70% of the database (Train dataset) to develop the five Random Forest algorithms, or models, shown in FIG. 20. Model 1, 2 and 5 are binary classifiers trained to detect ventilator disconnection, the presence of asynchronous breathing, and breath-stacking, respectively. Model 3 is a multiclass classifier trained to recognize signal morphology, including synchrony, variable breathing and the four types of asynchronies previously described. Model 4 is another multiclass classifier trained to predict epoch severity.

FIG. 20. depicts an exemplary embodiment of an exemplary schematic diagram showing various machine learning algorithms, according to an exemplary embodiment of the present invention, including an exemplary schematic showing the various machine learning algorithms, or models, trained with 70% of the database instances, according to an exemplary embodiment.

We used 30% of the database to test the performance of the various algorithms (Test dataset). Accuracy was defined as the fraction of correct predictions made by the algorithms using the Test dataset. We used the following standard metrics to evaluate individual model prediction: precision, or positive predictive value (PPV), sensitivity, specificity, and the fl-score, defined as the harmonic mean of precision and sensitivity.

Algorithm reliability was evaluated by comparing the predictions of Models 2,3 and 4 to those made by clinicians evaluating identical datasets. To that effect, we culled randomly 1000 instances from the Test data to create seven identical versions of a “Concordance dataset”. Four of these sets were given to junior clinicians not experienced in asynchrony identification (Naïve readers). Each was asked to score the Concordance dataset following a one-hour lecture on epoch classification. The remaining sets were given to classify by three individuals with considerable expertise in asynchrony identification (Expert readers).

Statistics. We used Cohen's kappa statistic (7) to compare the agreement in Concordance dataset predictions made by each reader and those of models 2, 3, and 4. One-sided Student's t-test was used to compare mean kappa statistic for the Expert and Naïve groups. Fleiss kappa statistic (8) was used to determine within-group agreement. The magnitude of agreement was assessed using the scale of Landis and Koch (9) for kappa statistic as poor (0 to 0.19), fair (0.20 to 0.39), moderate (0.40 to 0.59) and substantial (0.60 to 0.79). Chi-Square was used to test for differences in the percentages of epochs associated with the use of i.v. propofol. The Mann-Whitney test was used to compare the difference in percent time being asynchronous by survivors and decedents. Unless otherwise stated, data are shown as median [25%, 75% quartiles]. Additional information on epoch scoring and algorithm development may be found in the online data supplement.

### Results

Demographics and ventilator data: We enrolled 44 patients aged 62.5 [57.0, 70.8] years from February through December 2018. (Table 1E, online data supplement). All were medical patients, the majority were African-American and 50% were male. Most patients were intubated in response to a pulmonary condition (52.3%), including COPD, pneumonia and ARDS. Patients remained on ventilatory support for 3[2, 5] days with ICU and hospital stay of 6 [3,11] days and 15 [8, 26] days, respectively. Modes of ventilation used were pressure regulated volume control (PRVC; 61% of epochs), pressure control (PC; 31%), pressure support (6%) and volume control (2%).

Mortality: 28-day mortality rate for the entire cohort was 31.8%, a rate that compares favorably to the 44% predicted by their SAPS II scores of 54.5 [47.0, 63.8] (10). Decedents were asynchronous for 52.4% [41.0, 61.1] of their monitored time, compared to 33.7% [13.5, 45.4] for survivors (p=0.015). No differences were found in SAPS II scores between decedents and survivors of 59.5 [53.5, 67.0] and 51.5 [46.3, 60.8], respectively.

Asynchronies: We enrolled patients within 13 hours [6, 17; min=1; max=23] from the time they were placed on invasive ventilatory support. Each patient was monitored for 46 hours [24, 70; min 2; max 106] for a total cohort monitoring time of 2112 hours. We captured and classified 50,712 epochs, encompassing approximately 2.6 million breathing cycles. Variable breathing was the most common breathing pattern (36.5%). Asynchronous breathing accounted for 42.0% of the epochs and full synchrony 21.1% (Table 1).

All patients experienced asynchronies while monitored and were asynchronous for 39% [20, 59; min=4; max=100] of the time monitored. 57.9% of all asynchronous epochs were classified as premature or late cycling, followed by 20.4% as ineffective efforts, 13.2% as double triggering and 8.5% as complex. 25.3% of epochs classified as asynchronous displayed two types of asynchronies.

There were differences in the distribution of epoch severity. Only 7.4% of variable breathing epochs were graded severe, compared to 48.4% of all epochs. The highest percentages of asynchronous epochs graded as severe were among those classified as double triggering (78%) and complex (88%).

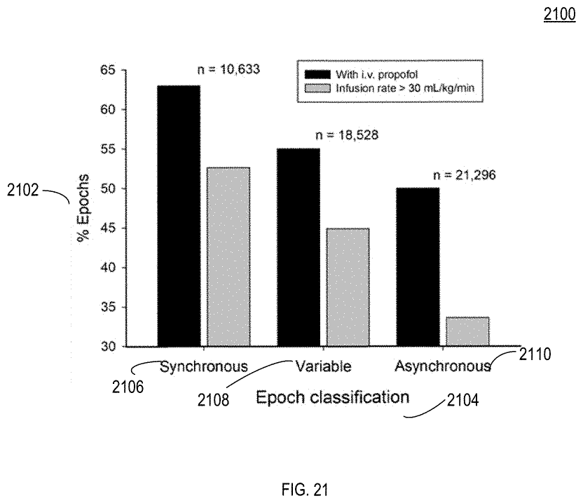

Sedative administration. A shown in FIG. 21, i.v. propofol administration was noted in 63% of synchronous epochs, compared to 55% for variable breathing and 50% for asynchronous epochs (p<0.001). A greater percentage of synchronous epochs (53%) were treated with a propofol infusion rate≥30 mcg·kg−1·min, compared to 45% and 34% for variable breathing and asynchronous epochs (p<0.001).

FIG. 21 depicts an exemplary embodiment of exemplary black bars showing the percentage of exemplary synchronous, variable breathing and asynchronous epochs associated with the use of i.v. propofol, according to an exemplary embodiment. According to an exemplary embodiment, the exemplary grey bars can represent the percentage of these epochs infused with propofol rates≥30 mcg·kg−1·min, according to an exemplary embodiment. A larger percentage of synchronous epochs were both associated with the use of propofol and infused at rates≥30 mcg·kg−1 (p<0.001 by Chi-Square), according to an exemplary embodiment. Asynchronous epochs had the lowest percentage of propofol use and i.v. infusion rate, according to an exemplary embodiment. The number of epochs in the groups are shown above each set of bars, according to an exemplary embodiment.

Algorithm development: Nineteen variables, or features, having ≥5% correlation with the various predicted outcomes were used to develop all five models. Ten of these variables were related to the frequency spectra of airway pressure and flow (online data supplement). The others were: FIO2, respiratory rate, tidal volume, minute ventilation, peak airway pressure, PEEP, the patient's age and the covariances of total breath cycle timing and of peak pressure. Neither ventilator mode nor primary diagnosis met the correlation requirement. Except for age, no other demographic variable appeared to improve model prediction.

Algorithm accuracy: Table 2 shows evaluation metrics for the five Random Forest models. Model 1 detected ventilator disconnection with 71% sensitivity and 100% specificity, a desirable characteristic for an alarm producing monitor. Model 2 detected asynchronous epochs with 91% accuracy and excellent values for sensitivity and specificity. Model 3 performed best when identifying synchronous breathing and worst at identifying complex asynchronies. Considering the relatively small percentage of some asynchronies in the database, asynchrony discrimination was reasonably good, with PPVs in the range of 70% to 84%. Model 4 performed best at identifying mild or severe epochs, with 94% and 82% precision, respectively, and Model 5 showed excellent accuracy in detecting breath-stacking.

Algorithm reliability: Table 3 and Table 4E show Cohen kappa statistics for agreement between each individual reader and the predictions of Models 2, 3 and 4 in classifying identical 1000-instance Concordance datasets. Also shown are the degrees of agreement between the models' predictions and the assessors' original classification of the 1000 instances culled from the Test dataset. The magnitude of the agreement between Naïve readers and all three models was as fair, rating best for asynchrony severity. Conversely, the agreement magnitude for the Expert readers was rated as moderate for all three models, being nearly substantial for Models 2 and 4. The kappa statistics for Expert readers were greater than those for the Naive readers (p=0.04; 0.02 and 0.04 for Models 2, 3 and 4, respectively). The models' predictions were in perfect agreement with the assessors' classifications for all three groups, with kappa values lying three standard deviations outside the mean for Expert readers (p<0.01). The Fleiss kappa statistics for inter-rater agreement for the Naïve group were approximately half of those of the Expert group for all three models (Table 5E).

### Discussion

The current standard for detecting asynchronous breathing consists of visually inspecting 30-minute long recordings of airway signals (11, 12). Quantification is based on the asynchrony index (AI), defined as the percent ratio of identified asynchronies to total respiratory rate. In addition to its post hoc nature, this subjective and labor-intensive method is low in sensitivity and positive predictive value (13).

Computer driven methods for asynchrony identification have focused mainly on breath-by-breath differentiation of normal from asynchronous breathing. One approach has been the formulation of algorithms comparing signal morphology to a priori defined mathematical descriptions of airway pressure and flow (14, 15, 16, 17, 18). Others have proposed algorithms derived with machine-learning techniques to identify specific types of asynchronies (19, 20, 21, 22). These monitoring approaches, while valid and providing clarity to our understanding of asynchronous breathing, do not address the important issues of reliability and clinical relevance.

Our approach to automatic asynchrony identification is fundamentally different from those described above. Instead of analyzing each breath cycle in search of asynchronies, we aimed to replicate the process of clinical decision-making. Expert clinicians classified 2.5-minute epochs based on the contextual evaluation of airway signals and other clinical data. In addition to asynchronous breathing, the assessors considered full synchrony, variable breathing and ventilator disconnect. Epochs were further evaluated for breath-stacking and graded in severity based on the assessors' perceived degree of airway signal disruption.

Our study made significant use of spectral analysis. Each spectrum was computed by applying the Fast Fourier Transform to airway signals sampled during 131 seconds (online data supplement). Since each epoch's duration was 150 seconds, the spectra provided a comprehensive assessment of signal behavior during most of the epoch's time-window. These frequency spectra are relatively insensitive to artifacts that may distort airway signals, such as strong heart palpitations and water present in the ventilator tubing. Further, being mainly a function of breath-cycle timing, the frequency spectrum's pattern is mostly independent of ventilatory mode. This is an important characteristic, since most patients were ventilated with two or more modes during the time monitored.

Variable breathing had the highest occurrence among all classified epochs, and most were graded as mild or moderate in severity. This is not surprising, since variable breathing likely approximates normal respiratory variability. We also noted that one fifth of the epochs were fully synchronous. A larger percentage of these epochs were associated with the use of propofol, and at a greater infusion rate, than variable breathing or asynchronous epochs. A possible interpretation is that full patient-ventilator synchrony may represent a reliable marker of patient over-sedation.

Epochs were considered asynchronous when displaying four or more asynchronies. Assuming a respiratory rate of 16 breaths per minute, we reasoned that the occurrence of four asynchronies during an epoch's time window is equivalent to an AI>10%, the accepted threshold for asynchronous breathing (11). As initially reported by Blanch et al (23), we found that all patients exhibited asynchronous breathing at one time or another during the time monitored.

Asynchronous breathing comprised 42% of all epochs, with cycling asynchrony being the most frequently used classification. Since 48% of asynchronous epochs were graded as severe, it follows that mechanical ventilation was inadequate during 20% of the monitored time. Although fewer in number, the vast majority of double triggering and complex epochs were graded in the severe category, suggesting that therapeutic interventions should be directed mainly at managing these asynchrony types.

Breath-stacking (24) was scored in association with other breathing patterns in 25% of all epochs. This observation is compatible with the majority of our cohort having a pulmonary condition, given that breath-stacking was noted during 32% of recording time in acute lung injury patients (25).

Also in agreement with the work of Blanch et al (23), we noted that patients asynchronous during a larger percentage of their monitored time experienced greater 28-day mortality. This retrospective finding should be interpreted with care. The study was not powered for 28-day mortality and we did not monitor all patients for their entire time on ventilatory support. It does stress, however, the importance of research directed at asynchrony monitoring and management.

We used relatively few variables to train our models, and except for age, all were derived from ventilator acquired data. Although the ensemble Random Forest algorithm performed optimally with our data, it does not preclude the possibility of other types of machine-learning algorithms performing just well when applied to a larger database (see the electronic data repository for algorithm comparison). Algorithm performance was rated as excellent for Models 1, 2, and 5 and very good for Models 3 and 4. The ability of Model 3 to detect full synchrony with high sensitivity and specificity is important, since these patients are known to experience worse outcomes (26). To a lesser degree, but still with high precision, Model 3 identified variable breathing, the most common pattern encountered in the study.

The algorithms showed good predictive values and excellent specificity in identifying asynchronous epochs. Sensitivity was fair to poor for double triggering and complex asynchronies, maybe a result of their low occurrence in the database. Model 4 identified Mild and Severe breathing patterns with high certainty, but performed less well in predicting Moderate severity, perhaps reflecting the discrepancy in scorer's opinions when assigning this level of severity.

Algorithm performance is usually judged by its accuracy, or number of correct predictions made on an unseen Test set. Whereas models trained with reliable data are likely to be both accurate and reliable, algorithms trained with unreliable data may be accurate, but totally unreliable. Artificial intelligence can minimize human bias by discerning relational patterns within a mass of information, but it is unable to insure model reliability

We addressed the issue of reliability by using identical datasets to compare the classifications made by models 2, 3 and 4 to those of clinicians with diverse experience in asynchrony classification. We reasoned that unreliable models would result in small and similar kappa statistics for both Expert and Naive groups. Instead, we found the Expert group to be in moderate to substantial agreement with predictions made by the models, whereas those for the Naïve group only rated as fair. Kappa values for the Expert group also were significantly greater than those for the Naive group. These observations support the reliability of our algorithms.

Our models are lacking in several respects. The database was derived from a single center and reflects our clinical practice, as evidenced by the use of PRVC and PC as preferred modes of ventilation, it. Given the ethnic composition of our patient population, only 18% of the individuals enrolled in the study were Caucasian, raising the possibility of ethnic imbalance bias (27). Assessors were at times uncertain regarding the classification of epochs displaying two asynchrony types and we did not account for all types of asynchronies, in particular flow asynchronies, given their low incidence in patients ventilated with Servo ventilators.

The present study demonstrates the feasibility of developing machine learning algorithms to evaluate breathing patterns during invasive mechanical ventilation. Model accuracy could be improved by increasing the size of the database and widening the range of breathing patterns allowed. These may include auto and reverse triggering, flow asynchronies and the combination of two different asynchronies in one epoch. We also must assure model transportability by incorporating into the database the experience of clinicians caring for mechanically ventilated patients in the United States and other countries.

Model reliability remains a most difficult challenge. The considerable variation in asynchrony classification, noted here and reported by others (28), reflects the divergence of opinions found in the medical literature (1,11,12,13, 29,30,31). It is likely, therefore, that interpreter variability and subjectivity will remain problematic until consensus is reached among thought-leaders on how to define patient-ventilator asynchrony (32). The formulation of standards on the identification and classification of asynchronies will greatly enhance model reliability and promote the development of machine-learning algorithms.

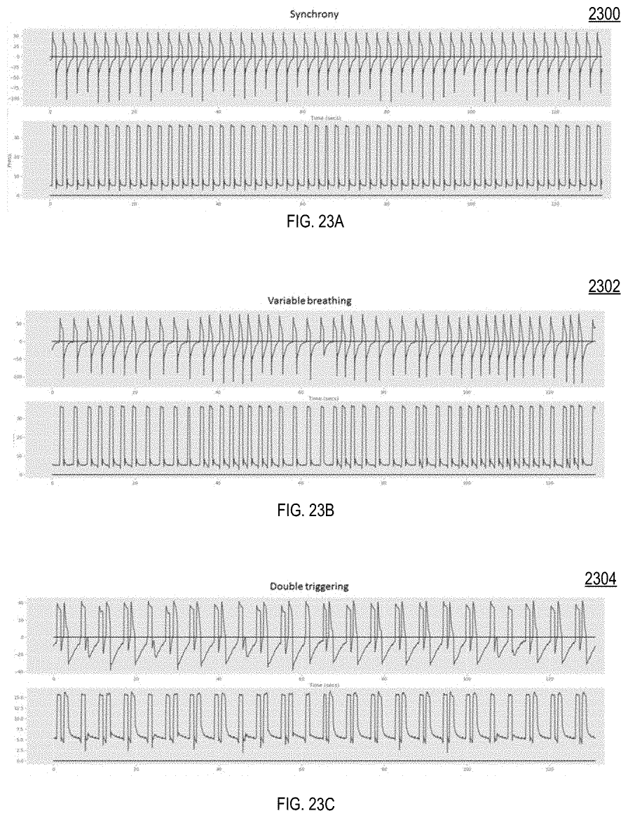

According to one exemplary embodiment, results of the exemplary intelligent monitoring application's machine learning algorithm based on analysis of the monitoring data of the mechanical ventilators, could be incorporated into an exemplary autonomous intelligent ventilator, according to an exemplary embodiment. An electronic monitor, according to one embodiment, can collect data, and send via, e.g., but not limited to, a WiFi or Bluetooth connection data from a coupled mechanical ventilator being monitored, along with any additional data captured, such as, e.g., any interventions that resulted in good outcomes, and together the data and good outcome interventions can be used and incorporated into an autonomous ventilator, according to an exemplary embodiment. According to one exemplary embodiment, a whole epoch can be reviewed, e.g., a 2.5 minute window, and in an exemplary continuous monitoring situation, can gather extensive 2.5 minute windows, day and night, according to an exemplary embodiment, rather than breath by breath, or breath cycle. The data can be looked at and can be matched to an exemplary 5 models, as illustrated in FIG. 23, according to an exemplary embodiment. Then using machine learning, and a large number of epochs, say, e.g., 50,000, an ML/AI model can overtime be found to be able to recommend the proper amount of sedation to achieve the positive outcomes desired. According to an exemplary embodiment, a vector of results can be obtained from looking at a group of experts, using an exemplary 19 variable matrix, and machine learning/artificial intelligence algorithms review approximately 1000 epochs, according to an exemplary embodiment. The test epochs of the ML results in testing, were found to correlate better with the expert clinicians, than the naïve clinicians, indicating the ML approach was effective, and worth further work, according to an exemplary embodiment. The exemplary algorithms were written in C++ or Python, according to an exemplary embodiment. Previously the monitoring device, coupled to the mechanical ventilator was hoped to encourage the care giver/nurse to do what the monitor recommended, however, unfortunately, the nurse did not always do what the nurse was supposed to do, e.g., the nurse might not be paying attention, or might be on break, or might not want to be bothered to change sedation based on the monitor's suggestion, but by using an automated and coupled, e.g., by WiFi of the raspberry pi controlled monitor, data from many good outcomes can be combined, and can identify the type of actuation helpful to bring about the kind of outcomes desired, and the system can continually improve by using iterative feedback to improve over time, and the intelligent, autonomous ventilator, according to an exemplary embodiment, can use such ML data to autonomously monitor a patient on a ventilator, and to provide better patient outcomes.

TABLE 1
Distribution of epoch's patterns and
severity, as classified by five experts.

| | | Severity | | |
|---|---|---|---|---|
| | % of | Mild | Moderate | Severe |
| Total epochs = 50,712 | total | (%) | (%) | (%) |
| Synchronous breathing | 21.0 | 100.0 | 0.0 | 0.0 |
| Variable breathing | 36.5 | 69.8 | 22.8 | 7.4 |
| Ventilator disconnect | 0.5 | 2.4 | 7.5 | 90.2 |
| Asynchronies: | | | | |
| Double Triggering | 5.5 | 6.5 | 15.4 | 78.1 |
| Ineffective Effort | 8.6 | 20.9 | 37.2 | 41.9 |
| Cycling | 24.3 | 33.7 | 28.4 | 37.9 |
| Complex | 3.6 | 2.7 | 9.1 | 88.2 |
| Asynchronies total: | 42.0 | 24.8 | 26.8 | 48.4 |

TABLE 2
Evaluation metrics for the different
Random Forest Classifier models (%)

| | Instances | | | | |
|---|---|---|---|---|---|
| | in data- | | Sensi- | Speci- | f1- |
| | set (%) | PPV | tivity | ficity | score |
| Model 1 (Accuracy 100%) | | | | | |
| Presence of disconnection | | | | | |
| No | 99.5 | 99.9 | 100 | 71 | 100 |
| Yes | 0.5 | 89 | 71 | 100 | 77 |
| Model 2 (Accuracy 91%) | | | | | |
| Presence of asynchrony | | | | | |
| No | 58.2 | 92 | 92 | 89 | 92 |
| Yes | 41.8 | 86 | 89 | 92 | 89 |
| Model 3 (Accuracy 82%) | | | | | |
| Epoch identification | | | | | |
| Synchronous breathing | 21.1 | 92 | 91 | 97 | 91 |
| Variable breathing | 36.7 | 80 | 87 | 87 | 83 |
| Asynchronies: | | | | | |
| Cycling | 24.4 | 77 | 81 | 92 | 79 |
| Ineffective Effort | 8.6 | 83 | 67 | 99 | 74 |
| Double Triggering | 5.6 | 84 | 64 | 99 | 73 |
| Complex | 3.6 | 70 | 52 | 100 | 59 |
| Model 4 (Accuracy 87%) | | | | | |
| Epoch Severity | | | | | |
| Mild | 60 | 94 | 94 | 89 | 94 |
| Moderate | 19 | 72 | 69 | 94 | 70 |
| Severe | 21 | 82 | 85 | 95 | 84 |
| Model 5 (Accuracy 93%) | | | | | |
| Breath-stacking detection | | | | | |
| No | 74.6 | 95 | 97 | 83 | 96 |
| Yes | 25.4 | 90 | 83 | 97 | 86 |

PPV = Positive predictive value

TABLE 3
Kappa statistics comparing individual and within
group reliability for the Concordance dataset

| | Model 2 | Model 3 | |
|---|---|---|---|
| | Asynchrony | Asynchrony | Model 4 |
| | (Yes/No) | Type | Severity |
| | Cohen's kappa for agreement between | | |
| | individual reader classification of the | | |
| | Concordance dataset and Models 2, 3 and 4 | | |
| Naïve readers | 0.29 ± 0.18 | 0.25 ± 0.09 | 0.38 ± 0.15 |
| (n = 4) | | | |
| Expert readers (n = 3) | 0.58 ± 0.08 * | 0.46 ± 0.10 * | 0.59 ± 0.11 * |
| Original classification | 0.97 † | 0.91 † | 0.95 † |
| | Fleiss' kappa statistic for within | | |
| | members of each group of readers in | | |
| | classifying the Concordance dataset | | |
| Naïve readers | 0.38 | 0.21 | 0.22 |
| (n = 4) | | | |
| Expert readers (n = 3) | 0.53 | 0.44 | 0.43 |

* p < 0.05 comparing Naïve to Expert readers
† p < 0.01 comparing original classification to Expert readers.

Further references to be considered, relevant for further study, include the following:

### REFERENCES

1. Epstein S K. How often does patient-ventilator asynchrony occur and what are the consequences? Respir Care 2011;56:25-38.

2. Gilstrap D, MacIntyre N. Patient-ventilator interactions. Implications for clinical management. Amer J Respir Crit Care Med 2013;188:1058-1068.

3. Gutierrez G, Ballarino G J, Turkan H, Abril J, De La Cruz L, Edsall C, George B, Gutierrez S, Jha V, Ahari J. Automatic detection of patient-ventilator asynchrony by spectral analysis of airway flow. Crit Care 2011;15:R167.

4. Gutierrez G, Williams J, Alrehaili GA, McLean A, Pirouz R, Amdur R, Jain V, Ahari J, Bawa A, Kimbro S. Respiratory rate variability in sleeping adults without obstructive sleep apnea. Physiol Rep 2016;4. pii: e12949.

5. Kallet R H, Luce J M. Detection of patient-ventilator asynchrony during low tidal volume ventilation, using ventilator waveform graphics. Respir Care 2002;47:183-185

6. Dres M, Rittayamai N, Brochard L. Monitoring patient-ventilator asynchrony. Curr Opin Crit Care 2016;22:246-253.

7. Cohen J. A coefficient of agreement for nominal scales”. Educational and Psychological Measurement 1960;20: 37-46.

8. Fleiss J L. Measuring nominal scale agreement among many raters. Psychological Bulletin 1971;76: 378-382.

9. Landis J R, Koch G G. The measurement of observer agreement for categorical data. Biometrics 1977;33: 159-174.

10. Le Gall J R, Neumann A, Hemery F, Bleriot J P, Fulgencio J P, Garrigues B, Gouzes C, Lepage E, Moine P, Villers D. Mortality prediction using SAPS II: an update for French intensive care units. Crit Care 2005;9:R645-652.

11. Thille A, Rodriguez P, Cabello B, Lellouche F, Brochard L. Patient-ventilator asynchrony during assisted mechanical ventilation. Intensive Care Med 2006,32:1515-1522.

12. Georgopoulos D, Prinianakis G, Kondili E. Bedside waveforms interpretation as a tool to identify patient-ventilator asynchronies. Intensive Care Med 2006;32:34-47.

13. Colombo D, Cammarota G, Alemani M, Carenzo L, Barra F L, Vaschetto R, Slutsky A S, Della Corte F, Navalesi P. Efficacy of ventilator waveforms observation in detecting patient-ventilator asynchrony. Crit Care Med 2011;39:2452-2457.

14. Younes M, Brochard L, Grasso S, Kun J, Mancebo J, Ranieri M, Richard J C, Younes H. A method for monitoring and improving patient: ventilator interaction. Intensive Care Med. 2007;33:1337-1346.

15. Mulqueeny Q, Ceriana P, Carlucci A, Fanfulla F, Delmastro M, Nava S. Automatic detection of ineffective triggering and double triggering during mechanical ventilation. Intensive Care Med 2007;33:2014-2018.

16. Chen C W, Lin W C, Hsu C H, Cheng K S, Lo C S. Detecting ineffective triggering in the expiratory phase in mechanically ventilated patients based on airway flow and pressure deflection: feasibility of using a computer algorithm. Crit Care Med 2008;36:455-461.

17. Blanch L, Sales B, Montanya J, Lucangelo U, Garcia-Esquirol O, Villagra A, Chacon E, Estruga A, Borelli M, Burgueño M J, Oliva J C, Fernandez R, Villar J, Kacmarek R, Murias G. Validation of the Better Care® system to detect ineffective efforts during expiration in mechanically ventilated patients: a pilot study. Intensive Care Med 2012;38:772-780.

18. Adams J Y, Lieng M K, Kuhn B T, Rehm G B, Guo E C, Taylor S L, Delplanque J P, Anderson N R. Development and Validation of a Multi-Algorithm Analytic Platform to Detect Off-Target Mechanical Ventilation. Sci Rep 2017;7:14980.

19. Mulqueeny Q, Redmond S J, Tassaux D, Vignaux L, Jolliet P, Ceriana P, Nava S, Schindhelm K, Lovell N H. Automated detection of asynchrony in patient-ventilator interaction. Conf Proc IEEE Eng Med Biol Soc 2009;2009:5324-5327.

20. Sottile P D, Albers D, Higgins C, Mckeehan J, Moss M M. The Association Between Ventilator Dyssynchrony, Delivered Tidal Volume, and Sedation Using a Novel Automated Ventilator Dyssynchrony Detection Algorithm. Crit Care Med 2018;46:e151-157

21. Gholami B, Phan T S, Haddad W M, Cason A, Mullis J, Price L, Bailey J M. Replicating human expertise of mechanical ventilation waveform analysis in detecting patient-ventilator cycling asynchrony using machine learning. Comput Biol Med 2018;97:137-144.

22. Loo N L, Chiew Y S, Tan C P, Arunachalam G, Ralib A M, Mat-Nor M B. A machine learning model for real-time asynchronous breathing monitoring. IFAC-PapersOnLine 2018;51; 378-383.

23. Blanch L, Villagra A, Sales B, Montanya J, Lucangelo U, Luján M, Garcia-Esquirol O, Chacón E, Estruga A, Oliva J C, Hernández-Abadia A, Albaiceta GM, Fernández-Mondejar E, Fernández R, Lopez-Aguilar J, Villar J, Murias G, Kacmarek RM. Asynchronies during mechanical ventilation are associated with mortality. Intensive Care Med 2015;41:633-641.

24. Beitler J R, Sands S A, Loring S H, et al. Quantifying unintended exposure to high tidal volumes from breath-stacking dyssynchrony in ARDS: the BREATHE criteria. Intensive Care Med 2016;42:1427-1436.

25. Pohlman M C, McCallister K E, Schweickert W D, Pohlman A S, Nigos C P, Krishnan J A, Charbeneau J T, Gehlbach B K, Kress J P, Hall J B. Excessive tidal volume from breath stacking during lung-protective ventilation for acute lung injury. Crit Care Med 2008;36:3019-3023.

26. Gutierrez G, Das A, Ballarino G, Beyzaei-Arani A, Türkan H, Wulf-Gutierrez M, Rider K, Kaya H, Amdur R. Decreased respiratory rate variability during mechanical ventilation is associated with increased mortality. Intensive Care Med 2013;39:1359-1367.

27. Rajkomar A, Hardt M, Howell M D, Corrado G, Chin M H. Ensuring Fairness in Machine Learning to Advance Health Equity. Ann Intern Med. 2018;169:866-872.

28. Ramirez I I, Arellano D H, Adasme R S, Landeros J M, Salinas F A, Vargas A G, Vasquez F J, Lobos I A, Oyarzun M L, Restrepo R D. Ability of ICU Health-Care Professionals to Identify Patient-Ventilator Asynchrony Using Waveform Analysis. Respir Care 2017;62:144-149.

29. Liao K M, Ou C Y, Chen C W. Classifying different types of double triggering based on airway pressure and flow deflection in mechanically ventilated patients. Respir Care 2011;56:460-466.

30. Branson R D, Blakeman T C, Robinson B R H. Asynchrony and dyspnea. Respir Care 2013;58:973-989.

31. Murias G, Lucangelo U, Blanch L. Patient-ventilator asynchrony. Curr Opin Crit Care 2016;22:53-59

32. Mireles-Cabodevila E, Dugar S. On the Need for Standard Definitions and Education to Optimize Patient-Ventilator Interactions. Respir Care 2017;62:248-249.

TABLE 1E
Patient Demographics and Admission Data

| Demographics: | | |
|---|---|---|
| Age (Years) | 62.5 | [57.0, 70.8] |
| Gender | 50% male | |
| Ethnicity (%): | | |
| Black | 68.2 | |
| Caucasian | 18.2 | |
| Hispanic | 11.4 | |
| Asian | 2.3 | |
| Risk factors: | | |
| Smoker | 22.7% | |
| Alcohol abuse | 20.5% | |
| IV drug user | 18.2% | |
| COPD | 13.6% | |
| Admission diagnosis: | | |
| Pulmonary | 52.3% | |
| Sepsis or septic shock | 18.2% | |
| Neurologic | 15.9% | |
| Cardiac | 4.5% | |
| Gastroenterological | 4.5% | |
| Oncological | 2.3% | |
| Renal | 2.3% | |
| Reason for intubation: | | |
| Airway protection | 45.5% | |
| Hypoxemia | 34.1% | |
| Hypercapnia | 20.5% | |
| Other admission data: | | |
| SOFA | 7.0 | [6, 9] |
| SAPS | 54.5 | [47.0, 63.8] |
| Glasgow scale* | 7 | [6, 9] |
| BMI (kg/m²) | 28.9 | [25.9, 36.0] |

*Ventilator modified Glasgow scale with a maximum of 10 points.

TABLE 2E
Epoch classification for enrollees according to asynchrony type

| Patient | | Variable | Double | Ineffective | | | | Monitored | # of | % Time |
|---|---|---|---|---|---|---|---|---|---|---|
| Number | Synch | Breathing | Triggering | Efforts | Cycling | Complex | Disconnect | Time (hr) | Asynchs | Asynch |
| 1820022301 | 0 | 80 | 56 | 6 | 341 | 13 | 1 | 20.7 | 416 | 84% |
| 1820022601 | 207 | 588 | 17 | 1 | 272 | 9 | 2 | 45.7 | 299 | 27% |
| 1820030701 | 414 | 595 | 49 | 4 | 22 | 23 | 5 | 46.3 | 98 | 9% |
| 1820030801 | 5 | 1 | 3 | 35 | 0 | 1 | 0 | 1.9 | 39 | 87% |
| 1820030802 | 102 | 638 | 6 | 1 | 354 | 22 | 2 | 46.9 | 383 | 34% |
| 1820030803 | 0 | 0 | 0 | 1 | 54 | 0 | 0 | 2.3 | 55 | 100% |
| 1820030804 | 35 | 273 | 29 | 55 | 108 | 8 | 1 | 21.2 | 200 | 39% |
| 1820031601 | 197 | 854 | 49 | 397 | 470 | 277 | 9 | 93.9 | 1193 | 53% |
| 1820031602 | 689 | 318 | 42 | 3 | 546 | 47 | 9 | 68.9 | 638 | 39% |
| 1820032001 | 413 | 37 | 30 | 536 | 65 | 55 | 11 | 47.8 | 686 | 60% |
| 1820032101 | 143 | 43 | 6 | 8 | 301 | 6 | 5 | 21.3 | 321 | 63% |
| 1820032201 | 927 | 663 | 3 | 9 | 16 | 43 | 6 | 69.5 | 71 | 4% |
| 1820032301 | 80 | 0 | 7 | 4 | 3 | 7 | 1 | 4.3 | 21 | 21% |
| 1820032401 | 417 | 138 | 6 | 30 | 35 | 13 | 2 | 26.7 | 84 | 13% |
| 1820033101 | 28 | 521 | 1 | 0 | 25 | 24 | 3 | 25.1 | 50 | 8% |
| 1820040101 | 5 | 851 | 8 | 59 | 1101 | 24 | 9 | 85.7 | 1192 | 58% |
| 1820040201 | 0 | 404 | 1 | 32 | 137 | 33 | 2 | 25.4 | 203 | 33% |
| 1820040501 | 393 | 195 | 0 | 20 | 22 | 24 | 0 | 27.3 | 66 | 10% |
| 1820040502 | 459 | 517 | 9 | 13 | 87 | 32 | 3 | 46.7 | 141 | 13% |
| 1820040601 | 680 | 490 | 10 | 2 | 620 | 7 | 8 | 75.7 | 639 | 35% |
| 1820041301 | 217 | 760 | 11 | 24 | 88 | 26 | 6 | 47.2 | 149 | 13% |
| 1820042401 | 0 | 214 | 2 | 3 | 368 | 44 | 0 | 26.3 | 417 | 66% |
| 1820042701 | 303 | 149 | 14 | 5 | 16 | 8 | 6 | 20.9 | 43 | 9% |
| 1820050801 | 75 | 229 | 66 | 25 | 91 | 48 | 2 | 22.3 | 230 | 43% |
| 1820051401 | 65 | 304 | 5 | 104 | 108 | 13 | 3 | 25.1 | 230 | 38% |
| 1820051501 | 243 | 935 | 24 | 15 | 344 | 59 | 23 | 68.5 | 442 | 27% |
| 1820053001 | 534 | 317 | 136 | 212 | 825 | 161 | 10 | 91.5 | 1334 | 61% |
| 1820061301 | 0 | 917 | 313 | 31 | 338 | 61 | 6 | 69.4 | 743 | 45% |
| 1820071201 | 216 | 154 | 32 | 0 | 313 | 4 | 3 | 30.1 | 349 | 48% |
| 1820071801 | 279 | 281 | 11 | 28 | 27 | 18 | 6 | 27.1 | 84 | 13% |
| 1820072401 | 74 | 318 | 40 | 1 | 50 | 27 | 4 | 21.4 | 118 | 23% |
| 1820073101 | 0 | 34 | 4 | 434 | 12 | 0 | 2 | 20.3 | 450 | 93% |
| 1820080101 | 18 | 218 | 1 | 274 | 1692 | 229 | 14 | 101.9 | 2196 | 90% |
| 1820081601 | 352 | 1292 | 32 | 86 | 712 | 32 | 9 | 104.8 | 862 | 34% |
| 1820082201 | 410 | 615 | 35 | 728 | 262 | 19 | 32 | 87.5 | 1044 | 50% |
| 1820082801 | 163 | 26 | 9 | 230 | 55 | 15 | 0 | 20.8 | 309 | 62% |
| 1820090401 | 124 | 281 | 149 | 252 | 813 | 44 | 6 | 69.5 | 1258 | 75% |
| 1820091301 | 88 | 418 | 1478 | 11 | 407 | 124 | 11 | 105.7 | 2020 | 80% |
| 1820103101 | 490 | 179 | 36 | 201 | 169 | 36 | 0 | 46.3 | 442 | 40% |
| 1820110101 | 183 | 1085 | 0 | 0 | 346 | 54 | 4 | 69.7 | 400 | 24% |
| 1820110801 | 426 | 885 | 31 | 120 | 115 | 25 | 11 | 67.2 | 291 | 18% |
| 1820111601 | 703 | 1355 | 43 | 150 | 129 | 35 | 12 | 101.1 | 357 | 15% |
| 1820121101 | 89 | 209 | 0 | 188 | 150 | 33 | 3 | 28.0 | 371 | 55% |
| 1820121201 | 387 | 147 | 9 | 2 | 325 | 26 | 3 | 37.5 | 362 | 40% |
| | | | | | | | | MEAN | VALUES | |
| | No | TI | DT | IE | DC | CM | DX | Time (hr) | Asynchs | % Time |
| Total | 10633 | 18528 | 2813 | 4340 | 12334 | 1809 | 255 | 48.0 | 484.0 | 42.0% |
| | 21.1% | 36.7% | 5.6% | 8.6% | 24.4% | 3.6% | 0.5% | 30.2 | | |

Modes of Ventilation

| | | | | | | | |
|---|---|---|---|---|---|---|---|
| Total | 50712 | | Total Epochs = 50,712 | Median | 46.0 | 353.0 | 38.9% |
| PRVC | 30994 | 61.1% | minus DX = 50,457 | 1st Q | 24.4 | 135.3 | 20.0% |
| PC | 15769 | 31.1% | | 3rd Q | 69.5 | 638.8 | 59.3% |
| VC | 1054 | 2.1% | | min | 1.9 | 21.0 | 4.3% |
| PSPC | 2895 | 5.7% | | max | 105.7 | 2196.0 | 100.0% |

### Machine Learning Algorithms

We used scikit-learn, an open source machine learning library for the Python programming language, to develop the classification supervised machine learning algorithms. We tested several algorithms to determine which proved to be the best predictor. They were Random Forest, k-Nearest Neighbors, Convolutional Neural Networks, Quadratic Discriminant Analysis, Logistic Regression, Gradient Boosted Decision Trees, Ada Boost, Naïve Bayes Classifiers and Support Vector Machines. For the purpose model development, the instances were distributed randomly into a Train data set encompassing 70% of the instances. The remaining instances were assigned to a Test data set and used to evaluate model performance.

TABLE 3E
Overall accuracies of different machine-learning algorithms
when applied to the Train dataset for the five models

| | Model 1 | Model 2 | Model 3 | | Model 5 |
|---|---|---|---|---|---|
| | Discon- | Asyn- | Asyn- | | Breath- |
| Classifier | nection | chrony | chrony | Model 4 | stacking |
| Algorithm | (Y/N) | (Y/N) | Identification | Severity | (Y/N) |
| Random Forest | 99.9 | 89.9 | 82.0 | 87.5 | 92.8 |
| k-Nearest | 99.7 | 85.3 | 57.7 | 80.6 | 88.7 |
| Neighbors, n = 3 | | | | | |
| Neural Networks | 99.9 | 81.2 | 68.5 | 0.80 | 87.2 |
| Quadratic | 97.5 | 73.3 | 57.7 | 75.1 | 42.9 |
| Discriminant | | | | | |
| Logistic | 99.8 | 75.9 | 57.7 | 79.6 | 87.1 |
| Regression | | | | | |
| Decision Trees | 96.3 | 79.7 | 64.8 | 81.1 | 87.5 |
| ADA Boost | 99.8 | 82.0 | 61.3 | 80.2 | 88.5 |
| Naïve Bayes | 96.5 | 74.4 | 58.2 | 72.7 | 70.4 |
| Support Vector | 99.7 | 58.2 | 40.5 | 57.3 | 73.8 |
| Machines | | | | | |

TABLE 4E
Cohen's kappa values
For the following table, all asynchrony types and variable
breathing are consolidated into a single group and compared
to synchronous breathing synchrony are consolidated into
another group. The classification becomes a binary variable
(synchrony vs. asynchrony).

| | | | |
|---|---|---|---|
| Observed | Expected | | |
| agreement | agreement | kappa | SE |

Each rater and original classification is compared with Model 2

| | | | | |
|---|---|---|---|---|
| Naïve 1 | 0.696393 | 0.550602 | 0.324413 | 0.030186 |
| Naïve 2 | 0.692385 | 0.514337 | 0.366607 | 0.031562 |
| Naïve 3 | 0.765531 | 0.52003 | 0.511492 | 0.031648 |
| Naïve 4 | 0.749499 | 0.501898 | 0.497089 | 0.031059 |
| Expert1 | 0.776553 | 0.507169 | 0.546606 | 0.031323 |
| Expert 2 | 0.844689 | 0.519398 | 0.676842 | 0.031643 |
| Expert 3 | 0.784569 | 0.549337 | 0.521969 | 0.030324 |
| Original | 0.969940 | 0.518343 | 0.93759 | 0.031632 |
| classification | | | | |

Comparing each rater and original classification with Model 3.
Asynchrony Type - Model 3

| | | | | |
|---|---|---|---|---|
| Naïve 1 | 0.406814 | 0.302967 | 0.148984 | 0.013621 |
| Naïve 2 | 0.396794 | 0.235134 | 0.211357 | 0.016565 |
| Naïve 3 | 0.487976 | 0.253916 | 0.313718 | 0.017651 |
| Naïve 4 | 0.493988 | 0.236899 | 0.336901 | 0.015678 |
| Expert1 | 0.57014 | 0.243069 | 0.432102 | 0.017142 |
| Expert 2 | 0.684369 | 0.255243 | 0.576196 | 0.017658 |
| Expert 3 | 0.533066 | 0.240404 | 0.385287 | 0.015406 |
| Original | 0.931864 | 0.260876 | 0.907815 | 0.017789 |
| classification | | | | |

Comparing each rater and original classification with Model 4.
Severity - Model 4

| | | | | |
|---|---|---|---|---|
| Naïve 1 | 0.633267 | 0.559216 | 0.167998 | 0.018418 |
| Naïve 2 | 0.743487 | 0.552694 | 0.426538 | 0.027498 |
| Naïve 3 | 0.781062 | 0.564261 | 0.497548 | 0.02537 |
| Naïve 4 | 0.732966 | 0.524614 | 0.438279 | 0.023833 |
| Expert1 | 0.822144 | 0.601231 | 0.553989 | 0.026245 |
| Expert 2 | 0.87976 | 0.573763 | 0.717902 | 0.027168 |
| Expert 3 | 0.82014 | 0.633104 | 0.50978 | 0.023394 |
| Original | 0.980461 | 0.581233 | 0.953341 | 0.026843 |
| classification | | | | |

TABLE 5E
Within group Inter-rater agreement (Fleiss kappa)

| | Naïve | Experts |
|---|---|---|
| | Readers(n = 4) | Readers (n = 3) |
| Asynchrony Scoring | | |
| Model 2 | | |
| Asynchrony (Yes/No) | 0.38 | 0.53 |
| Epoch identification | | |
| Model 3 | | |
| Synchronous breathing | 0.22 | 0.62 |
| Variable breathing | 0.11 | 0.37 |
| Double triggering | 0.47 | 0.63 |
| Ineffective effort | 0.25 | 0.58 |
| Cycling | 0.25 | 0.18 |
| Complex | 0.30 | 0.39 |
| Combined κ | 0.21 | 0.44 |
| Severity Scoring | | |
| Model 4 | | |
| Mild | 0.29 | 0.63 |
| Moderate | 0.02 | 0.21 |
| Severe | 0.36 | 0.37 |
| Combined κ | 0.22 | 0.43 |

### Data Acquisition Details

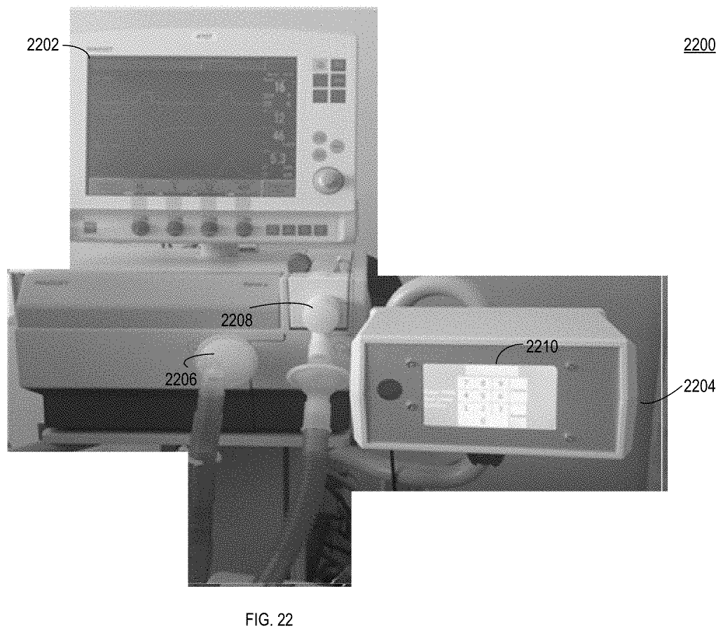

FIG. 22 depicts an image of an exemplary embodiment of a monitor 2204 including exemplary integrated display 2210, including ventilator hoses 2206 and 2208 coupled to a patient, and ventilator 2202 coupled electronically as discussed above with reference to FIG. 11 for exemplary monitoring and analysis functions; monitor 2204 is shown in schematic form in block diagram 1100 of FIG. 11, illustrated here coupled via various exemplary interfaces to a mechanical ventilator 2202, according to an exemplary embodiment of the present invention.

We acquired ventilator data continuously with a CS1 monitor (available from Respivar, LLC, Orrtanna, Pa. USA, see image of monitor 2204 coupled to a mechanical ventilator 2202, shown in image 2200 of FIG. 22). The monitor incorporates a Nucleo F787ZI microcontroller (https://os.mbed.com/platforms/ST-Nucleo-F767ZI/) connected to the ventilator's RS-232 port to interact with the Servo ventilator Computer Interface Emulator, according to an exemplary embodiment. Data gathering, according to an exemplary embodiment, can begin with the Nucleo microprocessor signaling the ventilator to acquire, in parallel, 4096 samples of the airway flow and pressure signals at 31.25 Hz, a rate satisfying the Nyquist-Shannon criterion to avoid aliasing of data, according to an exemplary embodiment. Once signal sampling was completed (131.07 seconds), according to an exemplary embodiment, frequency spectra for airway pressure and the inspiratory and expiratory phases of airway flow signals were computed by the microprocessor using, according to an exemplary embodiment, the Fast Fourier Transform (FFT) algorithm (Duhamel P, Vetterli M: Fast Fourier transforms: a tutorial review and a state of the art. Signal Processing 1990, 19:259-299). Spectral frequency granularity was 0.46 cycles per minute, according to an exemplary embodiment.

Algorithm comparison: We initially identified 33 variables related to patient demographics and ventilator data as model features. From these, we further selected 19 having ≥5% correlation with the predicted asynchronies. Application of the supervised machine learning algorithms to the Train data set resulted in the accuracy scores shown in Table 3e. The Random Forest algorithm yielded the highest accuracy score and Support Vector Machines the worst for all three models. The k-Nearest Neighbors and Convolutional Neural Networks and ADA Boost algorithms also performed well.

FIGS. 23A-23G depict various exemplary Examples of Epoch Classification, according to an exemplary embodiment.

Specifically, FIG. 23A illustrates an exemplary Synchrony example Epoch 2300, FIG. 23B illustrates an exemplary Variable Breathing example Epoch 2302, FIG. 23C illustrates an exemplary Double Triggering example Epoch 2304, FIG. 23D illustrates an exemplary Ineffecive Efforts example Epoch 2306, FIG. 23E illustrates an exemplary Cycling with breath stacking example Epoch 2308, FIG. 23F illustrates an exemplary Complex example Epoch 2310 including ineffective efforts, double triggering, and cycling, and FIG. 23G illustrates an exemplary Disconnect example Epoch 2312, according to an exemplary embodiment.

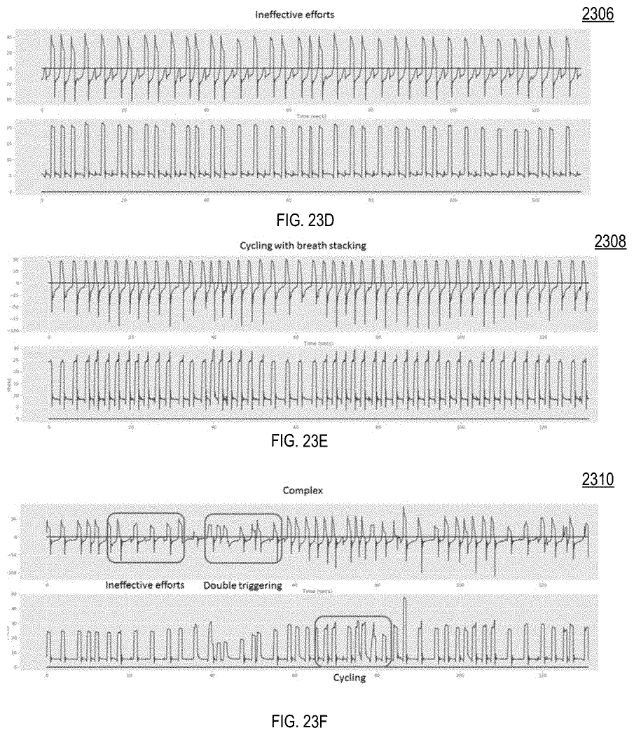

FIG. 24 depicts an exemplary embodiment of a diagram 3500 illustrating example formatting of data referenced in an accompanying appendix, according to an exemplary embodiment of the present invention.

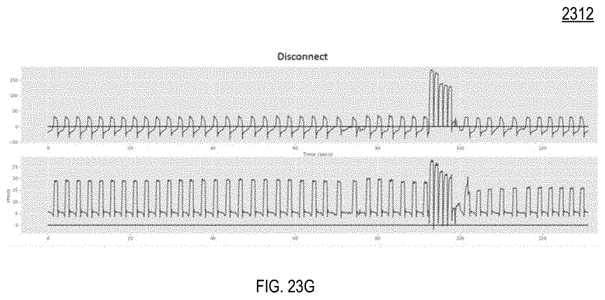

FIG. 25 depicts an exemplary embodiment of a diagram 3600 illustrating an example chart graphing average vs. difference for ration H1/DC accuracy testing, graphing differences between example Mbed and MATLAB H1/DC results, according to an exemplary embodiment of the present invention.

Obtaining an optimal level of sedation in mechanically ventilated patients is a major clinical challenge. Currently, the degree of sedation is determined by largely qualitative methods-sedation scales based on highly subjective clinical and visual patient assessment. Patient sedation needs can be more accurately assessed by determining the level of patient ventilator asynchrony (PVA), a condition where the patient's neural ventilator rhythm fails to coincide to that of the mechanical ventilator. Dr. Gutierrez of GW Medical Faculty Associates previously discovered a quantifiable method to determine PVA using spectral analysis of airflow signals. The objective of this project was to build a stand-alone monitoring device that incorporates Dr. Gutierrez's method to automatically, continuously, and noninvasively track PVA.

Our device is a 7.95×5.98×3.54 inch box weighing under 3 pounds with an enclosed Nucleo F767ZI mbed microcontroller, a 5V USB power source, and a 10.9 cm ULCD 43DT 4D Systems touch-sensitive screen display on the front of the box. The microcontroller was programmed with a C/C++ integrated development environment to acquire and process data from the Servo-i ventilator and to display data on the screen. The screen is menu-driven and current data is visible in a color-coded gauge and past data is accessible on one, twelve, and twenty-four hour history graphs.

Initial testing results indicate that the device's alarm system is accurate and that its algorithm produces values with a processing time of 60 milliseconds and errors of at most ±3.5%. 25 out of 25 users found the device easy to mount, and 100% could discern data on the default screen from 15 ft away. Shortcomings include unsatisfactory touch sensitivity with only 60% of users successful at pressing a button by the 2^{nd} attempt, and failure to provide a means to distinguish patients.

The monitor is an imaginative device that addresses a pertinent clinical problem and meets some of the clinical needs of ICU. Future development offers the opportunity for the device to communicate with other ventilator models, provide means of distinguishing patients via MRN, communicate with and store data from a capnograph and pulse oximeter, and meet all of the clinical needs of ICU.

There is an ideal range for the use of sedation medication in patients on mechanical ventilation. Most ventilated patients receive sedatives and analgesics to reduce pain and wakefulness, thereby reducing their risk of self-extubation [12]. On the other hand, oversedation can be harmful, as it can prevent the body from being able to breathe on its own and induce the patient into deep sleep. Overuse of sedatives has been shown to increase the duration of a patient's time spent under mechanical ventilation and in the ICU [12].

Sedative overuse is motivated by an erroneous idea. It is convention for clinicians to administer more sedatives when they find a patient's respiratory rate variability to be too high [3]. This is done to increase the synchrony of the ventilator's mechanical input with the patient's respiratory output. This thinking fails to recognize variability in breathing as a sign of good health. A healthy human exhibits a wide range of respiratory variability and it has been shown that patients with greater variability are more successful in weaning off their ventilators [14].

There are few efficient and quantitative approaches in monitoring patient sedative needs, and even fewer devices have been designed to accomplish this goal. The most widely used current method to assess a patient's respiratory variability rate is to calculate the Asynchrony Index (AI). This is derived from the real-time airway flow and pressure tracings provided by the ventilator [Dr. G 3]. The AI is the number of asynchronous breathing events within a certain period divided by the number of respiration cycles within that period [2]. This calculation relies on human observation, which is inherently imprecise, and leads to subjective sedative dispensing. It also encourages clinicians to simply increase a patient's dose when he exhibits a high index.

In an effort to provide a more automated, real-time, and accurate sedation assessment, Dr. Guillermo Gutierrez at The George Washington University Hospital devised an approach for monitoring sedation levels based on spectral analysis of ventilator respiratory signals. Spectral analysis can detect alterations in how periodic recurrent signals are, thus serving as a good tool for RRV measurements. Specifically, RRV can be assessed by using the amplitude ratio of the expiratory airway flow's first harmonic to the zero frequency component (DC) [3]. This ratio will henceforth be referred to as H1/DC.

This team set out to utilize Dr. Gutierrez's approach, creating a standalone device capable of implementing his algorithm and displaying the real-time results in a manner conducive to work in the hospital environment. This device, a respiration monitor, consists of 7.95×5.98×3.54 inch box with a touch screen and external memory drive accessible on its front side and inputs for the power and ventilator communication available on the back. A microcontroller, designated at CPU in FIG. 11, that sits inside the box draws the respiration signal of the patient from the ventilator, implements Dr. Gutierrez's algorithm and displays its results on the touch screen.

### Exemplary Design of Example Embodiments

In this section, we will first talk about the hardware of the device and its power subsystem, followed by a discussion on using the CPU to draw respiratory signals from the ventilator and to process the signals. Finally, we will explore the user interface subsystem, and the data export functionality of the device.

In order to create a stand-alone device, a fully encased box needed to be designed that could be implemented in the hospital setting. Pictures of the front and back of the finished box can be seen in FIGS. 12A and 12B, respectively. As previously mentioned, inputs for the power supply, ventilator, and external memory drive are accessible ports on the box. Clinicians may export data by inserting an SD card adapter into the slot on the front of the box, and by following the prompts on the screen. The microcontroller, portable power supply, and all other hardware is contained within the box (summarized in FIG. 11). The dock seen beneath the box in the photos can be attached to the top of the mechanical ventilator via a clamping mechanism. The box is placed in this dock and secured by, e.g., but not limited to, a simple VELCRO system.

This box was ordered from OkwUSA, Inc. Proven Process Medical Manufacturing offers cCGMP specification (Current Good Manufacturing Processes) on manufacturing plastic subcomponents according to FDA specifications for class II devices [9]. The device is considered a class II device because it is a monitoring device that will never directly interact with the patient. Machine grade polycarbonate was selected as a material for the box due to its impact resistance, good electrical characteristics, and ability to withstand high temperature sterilization practices. These considerations addressed the objective for the device to “maintain function in the hospital setting.”

An overall diagram describing power configuration in this device can be seen in FIG. 15. Because of the low voltage and controlled current into the device, the only requirement to meet hospital safety regulation standards was the inclusion of doubly insulated cords [1]. Key decisions in this subsystem included the selection of the 5V wall adapter, use of a battery that had pass-through charging to enable the simple series configuration to be maintained, and finally, feeding power into the USB communication port of the mbed. This power input is a temporary decision; the team found out late in the process that the Vin pin regulator requires between 7 and 12V to power the board. This configuration does allow the device to be used for an example eight hours without external power supply. This design decision met the objective that the device must be able to rely on its own power source for short periods of time, according to an exemplary embodiment.

The only subsystem external to the box is the ventilator communication. This communication of the mbed microcontroller with the Servo-i Computer Interface Emulator (CIE) requires an RS-232 serial interface at 9600 baud [11]. The RS-232 communication uses the 9600 8E1 serial protocol, meaning that the data is transmitted at 9600 baud with eight data bits, an even parity bit, and one stop bit [11]. Data collection code begins by formatting the RS-232 serial connection to meet the aforementioned requirements. This is accomplished with the call “vent.format(9, SerialBase: :Even, 1)”. Mbed documentation says that the first argument, number of data bits, should be a value between 6-8. However, it was found that including a parity bit overwrites the last data bit. The issue was solved by instead requesting 9 data bits which allowed for properly formatted commands to be transmitted. A UART for serial communication was then assigned to the ventilator.

Since the ventilator sends responses as ASCII strings, character buffers to store all of the to-be-collected data are also allocated. Pertinent settings information to be collected are respiratory rate and current time. In addition to this we also collect airway flow and airway pressure curves. These signals are transmitted in the form below seen in FIG. 24.

Since the signals are generated external to our device and must be constantly collected for the entirety of the time the device is on, a receive (RX) interrupt was written to collect them. This routine allows our device to use its processing power, outside the minor fraction that is committed to data collection, to continuously make other decisions and interact with the screen. See the code for a detailed description of the routine's logic. After all the above is set up, the main logic for the ventilator begins and a flow chart describing collection of ventilator data operations can be seen in FIG. 16. A tracker is used to control what functions should execute under the following conditions: more data is needed, data collection is done, and data is still being collected. The main while loop then begins. At the beginning of each collection cycle a check is sent to make sure the vent is not on standby. Once this check is passed, the code will then collect respiratory rate and date settings. A command will then be sent to request flow data from the ventilator. While collecting data, the code will maintain a loop of checking and updating the screen and checking for an external card for the two minutes that data is collected via the RX interrupt. Once data is collected, the control flag is changed to linearly execute data processing, gauge updating (with new H1/DC values from data processing), history graph updating, and exporting to the external card. The code will then repeat the loop starting at the standby check.

The data processing subsystem code begins by iterating through the airway flow signal and setting all positive values to zero to remove the inspiratory flow component of the signal. This is because the mechanical ventilator controls the inspiratory flow, while the patient controls the expiratory flow, so the expiratory flow is the component we are interested in [4]. Next, we perform the Fast Fourier Transform (FFT) to obtain the frequency spectrum of the expiratory flow signal. The FFT and related functions were provided to the team by Dr. Carl Wick.

Bit reversal is performed on the FFT output to order the output sequentially by increasing frequency. The real and imaginary components are squared and added together to obtain the magnitude of each point. The H1 and DC peaks are then obtained from these magnitudes. The DC peak is simply the first value in the output array representing zero frequency. The H1 peak should be at about the same frequency as the respiratory rate setting given by the ventilator. The neighborhood of the respiratory rate is searched and the highest peak is chosen as the H1 value. The final H1/DC value is then calculated, completing Dr. Gutierrez's algorithm [4].

The H1/DC is converted to a percentage to avoid issues with storing non-integer variables. This value is added to the queue to be displayed on the screen. Finally, the internal SD card is mounted and the data is appended to the file. For each 2.5 minute interval, one line is added to the file containing a tab-separated list of the timestamp, the current H1/DC value, the airway flow signal, and the FFT output. MRN will be added as another column in the file once it can be successfully collected.

The user interface subsystem is responsible for displaying the results of data processing to the clinician. It was designed to satisfy the objectives of “users can easily discern results”, “the device can be easily integrated into the hospital setting”, and the corresponding functions and specifications for these objectives. To implement the user interface, a 4D systems ULCD-43-DT was chosen in consideration of its advertised good touch screen sensitivity and easy to program GUI package. The resistive touch screen variant was chosen over the capacitive touch screen as it is expected that clinicians who will be using the device will often be wearing gloves that would prevent the capacitive screen from registering touches.To ensure that users could easily discern results, a gauge representing the H1/DC value is indicated by the position of the needle relative to the color-coded zones, with yellow corresponding to a patient being under synchronous, red as over synchronous, and green as ideally synchronous with their ventilator.

An mbed genie library is used to set the gauge with a new H1/DC value and to write to the LEDs on the default screen.

The “trend” button on the default screen (FIG. 17), H1/DC information from the queue/buffer is stored in an array of appropriate size for the specific time period. Then, for the past 12 hour and 24 hour graphs, every 4 and 6 consecutive H1/DC values for the past 12, 24 hours are averaged to fill arrays of 72 and 96 points, respectively. For the past hour graph, no averaging is conducted and 25 raw H1/DC values are plotted and magnified to stretch across the width of the scope. We only used averaging as it was necessary to condense information to fit the scope for the 12 and 24 hour graphs. The arrays of 25, 72, and 96 values are then converted to arrays of y-coordinate locations (shown by equation/ in the code on line).The scope runs from 15% to 75% and each H1/DC raw value or average below 15% is set to appear at the bottom of the scope while each value above 75% is set to appear at the top of the scope. The converted arrays are finally written to the scope objects using the function for display on the screen.

The device is memory includes data export functionality to offload 32 GB of patient data from the internal SD card to a USB mass storage device.

### Technical Assembly Instructions

Assembling Power

Gather:

1. Toggle Switch

2. ulcd 43DT Touchscreen

3. 4GB microSD Memory card for screen

4. Cablecc Down Angled 90 Degree STP UTP Ethernet Network Extension Cable 30 cm

5. CY 90 Degree Left Angled Micro USB Spin Male to USB B Female Panel Mount

6. Uninterrupted Power Bank Supply 7800 mah M312 11-13V DC UPS

7. Micro SD Breakout Board

8. Micro SD TO SD Card Extension Cable Adapter Flexible Extender SD/RS-MC/SDHC/MMC

9. Printed Circuit Board

10. OMNIHIL 12 Volt 2 Amp Power Adapter, AC to DC, Regulated 12V 2A

11. Two Pin 5 mm Pinch PCB Mount Screw Terminal Block Connector)

12. Male Header Pins for PCB

13. Adafruit 16 mm Panel Mount Momentary Pushbutton—Red [ADA1445]

14. SanDisk Ultra 32 GB microSDHC UHS-I Card with Adapter

### Instructions:

Connect these components as seen in the power diagram in FIG. 15.

### Software Assembly

Usage Instructions

Set up:

1.) Use clamp system to mount the monitor to an i.v. pole or use free standing. ventilator. Stand should be firmly mounted before proceeding.

2.) Use male d-sub9 connector from the ventilator to connect into female port on the back panel of the monitor.

3.) If device is uncharged, plug in to a standard 120V wall outlet. If the device is charged, you may proceed with or without plugging it in.

4.) Flip the switch on the back panel; check for the red indicator light and the home screen seen below to boot up. The monitor will detect data output by the ventilator and will display an H1/DC value on the screen gauge.

### Device Use:

1.) On the home screen reachable from any form, the user can see the gage displaying an indication that a patient might be under (yellow), ideally (green), or overly (red) sedated. User can only make a sedation decision to not give more sedative if it is indicated that the patient is over sedated.

2.) A user can also navigate to graphs of different intervals of data (past hour, past 12 hours, past 24 hours). This could help a user make more informed decisions when it comes to sedating a patient.

3.) Data can be exported by inserting the SD card adapter into the slot on the top right hand side of the front of the box. Watch the prompts on the screen to see when it is safe to remove the card.

### An Exemplary Computer Application Program to Exemplary Double Book MFA Clinic Slots Using Statistical Simulation

The aim of this project is to develop an interactive computer program capable of optimizing the use of GWU MFA outpatient clinics slots.

The problem:

Patients fail to show up for clinic appointments (so-called “No-shows”).
No-shows have a negative impact on:

Clinical income.

Clinic workflow.

Clinician's morale.

### Possible Solutions:

Charge a fee to patients who fail to show up.

ate a shadow schedule.

Double book all clinics with a fixed number of patients.

We (clinicians) need to differentiate between the “No show” rate and the clinic time utilization rate.

The no show rate is a patient related variable.

Depends largely on payer mix.

Difficult to change. i.e. phone reminders, financial incentives, etc.

On the other hand,

Clinic time utilization rate is a system's variable.

Clinic time slots are comparable to seats in an airplane.

They are amenable to manipulation.

The goal should be to fill as many clinic slots as possible without incurring a high cost.

Compression vs. Double Booking.

1. The cost of patient waiting?

2. The cost of harried clinic staff?

3. The cost of overworked clinicians?

Use of statistical simulation to develop a reliable model of clinic time utilization.

Build a stochastic model based on the known probabilities of:
A new (NPV) or a return (RPV) patient making an appointment.
The type of insurance they carry.
The likelihood of showing up to the appointment.

Once a reliable model is created, it can be used to forecast scenarios aimed at maximizing clinic time utilization.

Stochastic—Having a random probability distribution or pattern that may be analyzed statistically but may not be predicted precisely.

The model can use an approach that is analogous to, or similar to, airlines which may, from time to time, double book seats, according to one exemplary embodiment.

The model applies random numbers and known probabilities to forecast the composition of a given clinic according to the type of patient, new or return, as well as their type of insurances. It then determines whether the patients showed up, or not. It repeats this process thousands of times, until the results of the simulation equal the observed data.

Let's see how it works. To begin, we must have the no-show data according to type of patient and insurance type:

TABLE A1
No show data

| | | | Grand |
|---|---|---|---|
| No Show Report February YTD FY 19 | Arrived | No Show | Total |
| PANJRATH MD, GURUSHER | 435 | 93 | 528 |
| RETURN | 386 | 81 | 467 |
| MANAGED CARE | 102 | 19 | 121 |
| MEDICAID | 114 | 35 | 149 |
| MEDICARE | 168 | 27 | 195 |
| SELF-PAY | 2 | 0 | 2 |
| NEW | 49 | 12 | 61 |
| MANAGED CARE | 13 | 3 | 16 |
| MEDICAID | 18 | 7 | 25 |
| MEDICARE | 16 | 1 | 17 |
| SELF-PAY | 2 | 1 | 3 |

The process described above is again used to place an NPV or RPV in the double booked slots, figures out their insurance status and determines if they showed up or not. In this example they were given to a Medicaid patient, who did show up, and to a managed care patient who did not.

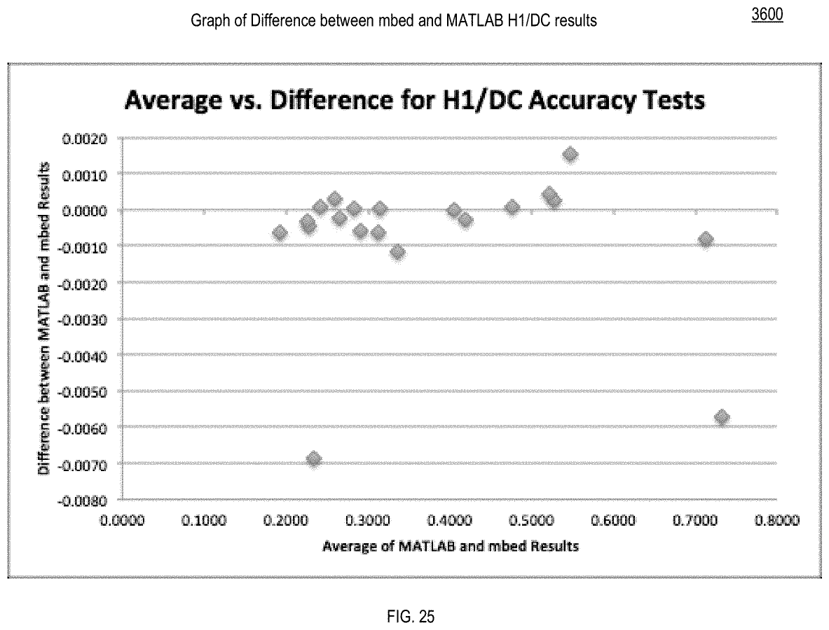

The algorithm is repeated for each clinic in the week for the number of weeks the clinic meets in a year, and then for as many years as one wishes, usually about 100, until the model statistics approach the ones shown in Table A1. The process is shown schematically in FIG. 27.

FIG. 27 depicts an exemplary Statistical Simulation Model 2700, according to an exemplary embodiment.

A computer program has been written using the Python programming language to read the input data from individual clinicians and model their clinics, according to an exemplary embodiment. The input data can include, according to an exemplary embodiment, exemplary text data files containing the following information:

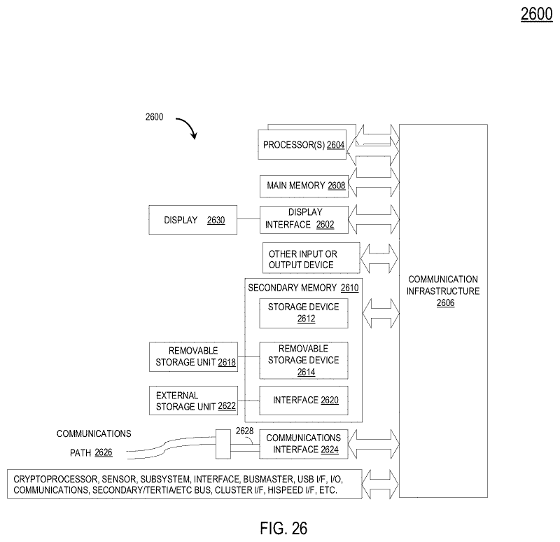

Shown in FIG. 28, is an exemplary model dashboard 2800 for this particular exemplary clinician (Dr. Panjrath), according to an exemplary embodiment.

The model allows the user to choose any combination of insurance types to double book. Up to six slots of each insurance type can be double booked. Ignoring double booking the “Self” insurance category (too few in numbers), this results in 145 possible combinations.

100 simulation-years of a clinic meeting four times weekly during 44 weeks in a year, results in the clinic being simulated 17,600 times. Searching for all possible double book combinations results in 2,552,000 clinic simulations.

To minimize computer time, a search strategy has been implemented to determine the optimal combination of slots that may be double-booked to produce the desired percent slot utilization with the fewer number of overbooked clinics.

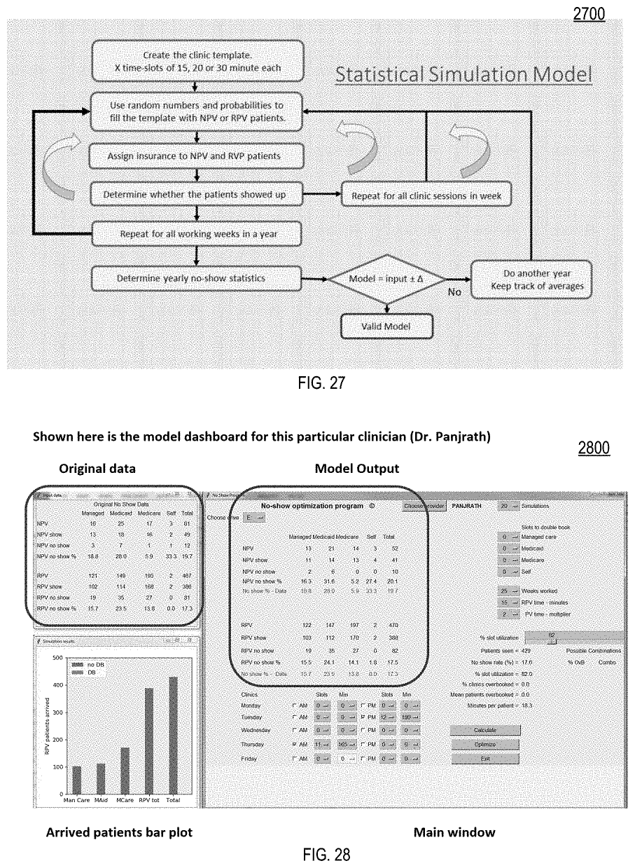

FIG. 29 depicts an exemplary model dashboard 2900 illustrating, for an example, exemplary overbooking occurs when more than 11 slots are needed to accommodate patients who showed up, according to an exemplary embodiment. An overbooked clinic is defined as one with more slots used than available in the clinic template. In the example 2900, overbooking occurs when more than 11 slots are needed to accommodate patients who showed up.

What has been learned:
It makes little difference which insurance group is used to double-book.
All insurance types benefit from double booking slots.
No need to keep track of habitual no shows.
Last minute cancellations are built into the no show statistics.
The model:
Determines % slot utilization and double booked sessions.
Is specific to clinicians.
Is dynamic. Implementation may be based on a rolling 12 months prior data.
It provides an excellent tool to assess clinic efficiency.

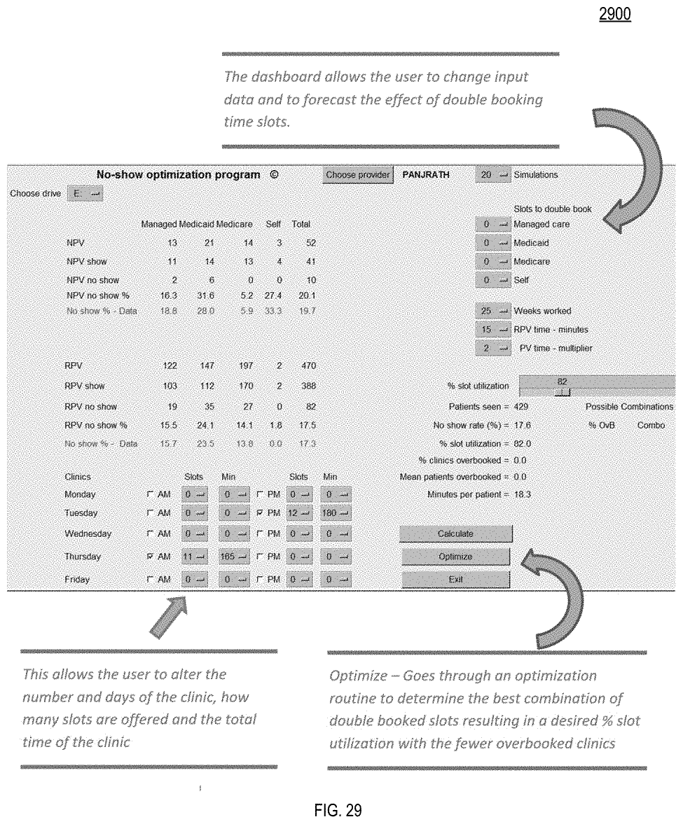

FIG. 30 depicts an exemplary chart 3000 of exemplary simulation results graphing an effect on arrived RPV of double booking (DB) one Medicaid slot, as is demonstrated, No patient group was negatively affected, and Same results were observed with managed care and Medicare, according to an exemplary embodiment.

A note on efficiency and patient satisfaction: Presently, many physicians double book their clinics and their clinic templates may bear little semblance to reality. The model can estimate the minutes spent for each clinic slot used. That number also should be taken into consideration when determining the number of slots to double book, since it will affect patient satisfaction.

IMPLEMENTATION. To implement the program, a number of steps are necessary.

Determine a strategy for each clinician that accounts for:
Clinic schedules.
Clinic templates.
Weeks in a year seeing patients.
No show data for prior year
Establish an acceptable number of overbooked clinics and time/slot.
Work with scheduling and the call center to develop a process.

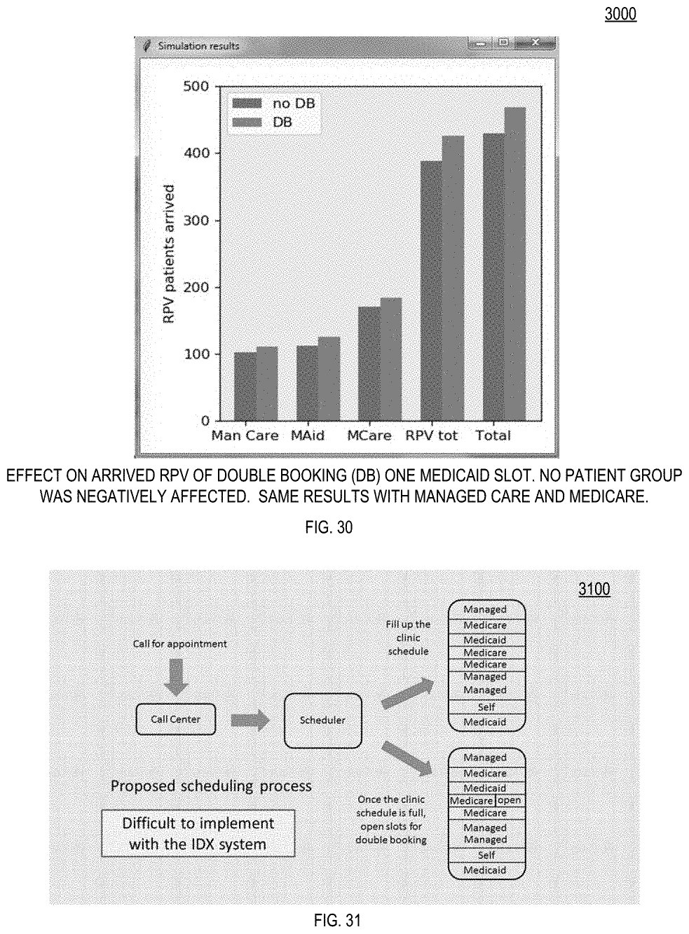

FIG. 31 depicts an exemplary block diagram 3100 of exemplary Proposed scheduling process, according to an exemplary embodiment.

PROPOSAL—Demonstration project—Exemplary Test Group—Pulmonary Division.
Develop a computer program to schedule patients.
Need: A dedicated scheduler located in the Pulmonary Division.
Call Center sends requests for appointments to the scheduler.
Patients are given appointments on a first come basis.
Once a session is full, the program opens specific RPV slots to double book.
The double booked slots are filled on a first come basis.
No-show statistical data are collected for each clinic session.
Determine % slot utilization rate and compare to historical data.
Project duration—six months.

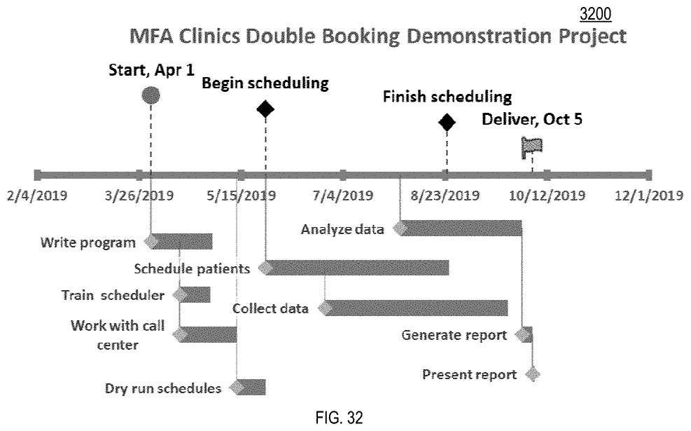

FIG. 32 depicts an exemplary project management GANNT chart of an exemplary rollout of an exemplary project to test efficacy of an exemplary double booking project in an exemplary clinic, according to an exemplary embodiment.

### An Exemplary Computer System Platform

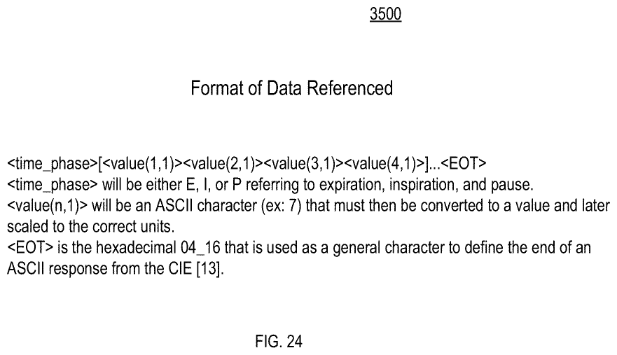

FIG. 26 depicts an exemplary computer system environment and/or platform, exemplary computing device platform as may be used for any of various devices of the various systems disclosed herein, according to one exemplary embodiment. For example, a clinician scoring interface as shown in FIG. 19 can be implemented on one or more machines as shown in exemplary computer architecture 1900. Similarly, an expert system, a central repository system, cloud storage, and/or server, and/or analysis device, and/or controller device for a monitor, and/or raspberry pi for an exemplary monitor, and/or mbed controller, a neural network, a machine learning system running any of various algorithms as discussed herein, e.g., running on an exemplary computer environment, such as, e.g., but not limited to, python environment, clinicians workstation, client, server, and/or scheduling server, or slot scheduling application, scheduling calendaring program, etc., any and all, can also be represented by exemplary block diagram 2600, in one exemplary embodiment.

For providing further understanding to assist in enabling the making and using of various exemplary embodiments of the claimed invention by a person having ordinary skill in the relevant art, certain computer analysis software and/or hardware platform equipment, which can be configured to provide an exemplary embodiment of computing system, which can be configured according to various exemplary embodiments to perform one or more functions including, receiving inputted data, and/or processing analysis of such inputted data, and/or providing output of such analyzed and processed data via one or more exemplary output devices capable of performing electronic automated computational analysis, including statistical algorithmic calculations.

FIG. 26 depicts an exemplary embodiment of a computer system 2600 that may be used in association with, in connection with, and/or in place of, but not limited to, computer platform 60, CPU, central processor, any of various elements depicted in, e.g., but not limited to, FIG. 1, FIG. 9, and/or FIG. 11, etc. according to exemplary embodiments of the present invention.

The present embodiments (or any part(s) or function(s) thereof) may be implemented using hardware, software, firmware, or a combination thereof and may be implemented in one or more computer systems or other processing systems. In fact, in one exemplary embodiment, the invention may be directed toward one or more computer systems capable of carrying out the functionality described herein. An example of a computer system 2600 is shown in FIG. 26, depicting an exemplary embodiment of a block diagram of an exemplary computer system useful for implementing the present invention. Specifically, FIG. 26 illustrates an example computer 2600, which in an exemplary embodiment may be, e.g., (but not limited to) a personal computer (PC) system running an operating system such as, e.g., (but not limited to) WINDOWS MOBILE™ for POCKET PC, or MICROSOFT® WINDOWS® NT/98/2000/XP/CE/,etc. available from MICROSOFT® Corporation of Redmond, Wash., U.S.A., SOLARIS® from SUN® Microsystems of Santa Clara, Calif., U.S.A., OS/2 from IBM® Corporation of Armonk, N.Y., U.S.A., MAC/OS, MAC/OSX , IOS, etc. from APPLE® Corporation of Cupertino, Calif., U.S.A., etc., or any of various versions of UNIX® (a trademark of the Open Group of San Francisco, Calif., USA) including, e.g., LINUX®, HPUX®, IBM AIX®, and SCO/UNIX®, etc. However, the invention may not be limited to these platforms. Indeed aspects of systems may include devices with various other input and/or output subsystems including, e.g., but not limited to, tablet displays, keyboards, various sensor(s), touch screen sensors, pressure sensors, location sensors (e.g., global positioning system (GPS), etc.), accelerometers, multi-dimensional sensor(s), temporal based datalogs, etc. Instead, the invention may be implemented on any appropriate computer system running any appropriate operating system. In one exemplary embodiment, the present invention may be implemented on a computer system operating as discussed herein. An exemplary computer system, computer 2600 is shown in FIG. 26. Other components of the invention, such as, e.g., (but not limited to) a computing device, a communications device, a telephone, a personal digital assistant (PDA), a personal computer (PC), a handheld PC, client workstations, thin clients, thick clients, proxy servers, network communication servers, remote access devices, client computers, server computers, peer-to-peer devices, tablets, touch-enabled devices, sensor enabled devices, location sensing devices, convertible, table/laptop, mobile, smart devices, smart phones, phablets, wearable technology, watch devices, glass devices, routers, web servers, data, media, audio, video, telephony or streaming technology servers, etc., may also be implemented using a computer such as that shown in FIG. 26 and/or additional subsystems perhaps not all shown, as discussed.

The computer system 2600 may include one or more processors, such as, e.g., but not limited to, processor(s) 2604. The processor(s) 2604 may be connected to a communication infrastructure 2606 (e.g., but not limited to, a communications bus, cross-over bar, or network, etc.). Alternatively one or more subsystems can be built into an exemplary integrated circuit (IC), and/or application specific integrated circuits (ASICs), field programmable gate arrays (FPGAs), etc. Various exemplary software embodiments may be described in terms of this exemplary computer system. After reading this description, it will become apparent to a person skilled in the relevant art(s) how to implement the invention using other computer systems and/or architectures.

Computer system 2600 may include a display interface 2602 that may forward, e.g., but not limited to, graphics, text, and other data, etc., from the communication infrastructure 2606 (or from a frame buffer, etc., not shown) for display on the display unit 2630.

The computer system 2600 may also include, e.g., but may not be limited to, a main memory 2608, random access memory (RAM), and/or a secondary memory 2610, etc. The secondary memory 2610 may include, for example, (but not limited to) a hard disk drive 2612, flash memory, a storage device, and/or a removable storage drive 2614, representing a floppy diskette drive, a magnetic tape drive, an optical disk drive, a compact disk drive CD-ROM, DVD, BLU-RAY, etc. The removable storage drive 2614 may, e.g., but not limited to, read from and/or write to a removable storage unit 2618 in a well known manner. Removable storage unit 2618, also called a program storage device or a computer program product, may represent, e.g., but not limited to, a floppy disk, magnetic tape, optical disk, compact disk, etc. which may be read from and written to by removable storage drive 2614. As will be appreciated, the removable storage unit 2618 may include a computer usable storage medium having stored therein computer software and/or data.

In alternative exemplary embodiments, secondary memory 2610 may include other similar devices for allowing computer programs or other instructions to be loaded into computer system 2600. Such devices may include, for example, a removable storage unit 2622 and an interface 2620. Examples of such may include a program cartridge and cartridge interface (such as, e.g., but not limited to, those found in video game devices), a removable memory chip (such as, e.g., but not limited to, an erasable programmable read only memory (EPROM), or programmable read only memory (PROM) and associated socket, flash memory, SDRAM, USB memory device, DVD-ROM, CD-ROM, BLU-RAY, etc., and other removable storage units 2622 and/or interfaces 2620 which may allow software and data to be transferred from the removable storage unit 2622 to computer system 2600.

Computer 2600 may also include an input device such as, e.g., (but not limited to) a mouse or other pointing device such as a digitizer, and a keyboard or other data entry device (none of which are labeled).

Computer 2600 may also include output devices, such as, e.g., (but not limited to) display 2630, touchscreen, passive, active, thin-film-transistor (TFT), passive touch, active touch, stylus enabled sensor based interface, flat panel, LCD, LED, and/or CRT, etc. and/or display interface 2602. Computer 2600 may include input/output (I/O) devices such as, e.g., (but not limited to) communications interface 2624, cable 2628 and communications path 2626, etc. These devices may include, e.g., but not limited to, a network interface card, and modems (neither are labeled). Communications interface 2624 may allow software and data to be transferred between computer system 2600 and external devices. Examples of communications interface 2624 may include, e.g., but may not be limited to, a modem, a network interface (such as, e.g., an Ethernet card), a communications port, a Personal Computer Memory Card International Association (PCMCIA) slot and card, Universal Serial Bus, Busmaster interface, PCI bus, interface bus, card slot, and/or other interface, firewire, USB 1, 2, 3, . . . n, A, B, C, D, . . . x, including any comparable interface released in the future, from a standards body, or the like, etc. Software and data transferred via communications interface 2624 may be in the form of signals 2628 which may be electronic, electromagnetic, optical or other signals capable of being received by communications interface 2624. These signals 2628 may be provided to communications interface 2624 via, e.g., but not limited to, a communications path 2626(e.g., but not limited to, a channel). This channel 2626 may carry signals 2628, which may include, e.g., but not limited to, propagated signals, and may be implemented using, e.g., but not limited to, wire or cable, fiber optics, a telephone line, a cellular link, a radio frequency (RF) link and other communications channels, etc.

In this document, the terms “computer program medium” and “computer readable medium” may be used to generally refer to media such as, e.g., but not limited to removable storage drive 2614, a hard disk installed in hard disk drive 2612, and signals 2628, etc. These computer program products may provide software to computer system 2600. The invention may be directed to such computer program products.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

In the following description and claims, the terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still co-operate or interact with each other.

An algorithm is here, and generally, considered to be a self-consistent sequence of acts or operations leading to a desired result. These include physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers or the like. It should be understood, however, that all of these and similar terms are to be associated with the appropriate physical quantities and are merely convenient labels applied to these quantities.

Unless specifically stated otherwise, as apparent from the following discussions, it is appreciated that throughout the specification discussions utilizing terms such as “processing,” “computing,” “calculating,” “determining,” or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

In a similar manner, the term “processor” may refer to any device or portion of a device that processes electronic data from registers and/or memory to transform that electronic data into other electronic data that may be stored in registers and/or memory. A “computing platform” may comprise one or more processors.

Embodiments of the present invention may include apparatuses for performing the operations herein. An apparatus may be specially constructed for the desired purposes, or it may comprise a general purpose device selectively activated or reconfigured by a program stored in the device, and/or a special purpose device programmed according to various algorithms and/or flowcharts and processes/methods as described at length herein.

Embodiments of the invention may be implemented in one or a combination of hardware, firmware, and software. Embodiments of the invention may also be implemented as instructions stored on a machine-readable medium, which may be read and executed by a computing platform, which may include one or more processors, such as, e.g., but not limited to, a microprocessor, a multi-core processor, a quadcore processor, a central processing unit (CPU), a quantum computer, a nanoprocessor, a computational engine, an information appliance, a virtual processor, a co-processor, a busmaster processor, a graphics processor (GPU), a digital signal processor (DSP), and/or other processor, to perform the operations described herein. A machine-readable medium may include any mechanism for storing or transmitting information in a form readable by a machine (e.g., a computer). For example, a machine-readable medium may include read only memory (ROM); random access memory (RAM); magnetic disk storage media; optical storage media; flash memory devices; electrical, optical, acoustical, magneto-optical, SD-RAM, SDCard, and/or other form of nontransitory medium storing any propagated signals (e.g., carrier waves, infrared signals, digital signals, etc.), and others.

Computer programs (also called computer control logic), may include object oriented computer programs, and may be stored in main memory 2608 and/or the secondary memory 2610 and/or removable storage units 2614, also called computer program products. Such computer programs, when executed, may enable the computer system 2600 to perform the features of the present invention as discussed herein. In particular, the computer programs, when executed, may enable the processor 2604 to provide a method to resolve conflicts during data synchronization according to an exemplary embodiment of the present invention. Accordingly, such computer programs may represent controllers of the computer system 2600.

In another exemplary embodiment, the invention may be directed to a computer program product comprising a computer readable medium having control logic (computer software) stored therein. The control logic, when executed by the processor 2604, may cause the processor 2604 to perform the functions of the invention as described herein. In another exemplary embodiment where the invention may be implemented using software, the software may be stored in a computer program product and loaded into computer system 2600 using, e.g., but not limited to, removable storage drive 2614, hard drive 2612 or communications interface 2624, etc. The control logic (software), when executed by the processor 2604, may cause the processor 2604 to perform the functions of the invention as described herein. The computer software may run as a standalone software application program running atop an operating system, or may be integrated into the operating system.

In yet another embodiment, the invention may be implemented primarily in hardware using, for example, but not limited to, hardware components such as application specific integrated circuits (ASICs), or one or more state machines, etc. Implementation of the hardware state machine so as to perform the functions described herein will be apparent to persons skilled in the relevant art(s).

In another exemplary embodiment, the invention may be implemented primarily in firmware.

In yet another exemplary embodiment, the invention may be implemented using a combination of any of, e.g., but not limited to, hardware, firmware, and software, etc.

Exemplary embodiments of the invention may also be implemented as instructions stored on a machine-readable medium, which may be read and executed by a computing platform to perform the operations described herein. A machine-readable medium may include any mechanism for storing or transmitting information in a form readable by a machine (e.g., a computer). For example, a machine-readable medium may include read only memory (ROM); random access memory (RAM); magnetic disk storage media; optical storage media; flash memory devices; electrical, optical, acoustical or nontransitory versions of other forms of previously propagated signals (e.g., carrier waves, infrared signals, digital signals, etc.), and others.

Still referring to FIG. 26, a universal integrated circuit card (UICC) (not shown) comprising a subscriber identity module and possibly a secure storage and/or cryptoprocessor can also coupled to the application or system processor. The system may further include a security processor (not shown) that may couple to the application or system processor or CPU.

One or more, or a plurality of sensors may couple to processor, or application processor to enable input of a variety of sensed information such as accelerometer and other environmental information. An audio, and/or video, output device may provide an interface to output sound, and/or other data, e.g., in the form of voice communications, played or streaming audio data and so forth.

As further illustration of FIG. 26, an exemplary near field communication (NFC) contactless interface can be provided in certain embodiments, that can communicate in a NFC near field via an NFC antenna, for example (not shown). While separate antennae can be used, not all are shown in FIG. 26 for simplicity of illustration, but will be apparent to those skilled in the relevant art, understand that in some implementations can include one or more antenna(ae) or a different set of antennae may be provided to enable various wireless functionality.

Further, an exemplary power management integrated circuit (PMIC) can couple to application processor, or system processor, to perform platform level power management. To this end, PMIC (not shown) may issue power management requests to application processor, system processor, etc., to enter certain low power states as desired. Furthermore, based on platform constraints, PMIC may also control the power level of other components of the exemplary system as shown in FIG. 26.

To enable communications to be transmitted and received, various circuitry may be coupled between an exemplary baseband or other system processor and/or an antenna (not necessarily shown in the block diagram). Specifically, a radio frequency (RF) transceiver and/or a wireless local area network (WLAN) transceiver, and/or a network interface card (NIC) may be present, in certain exemplary embodiments. In general, RF transceivers may be used to receive and transmit wireless data and calls according to a given wireless communication protocol such as, e.g., but not limited to, 3G, 4G, 5G, nG, next generation (NG), etc. wireless communication protocol such as in accordance with a code division multiple access (CDMA), global system for mobile communication (GSM), long term evolution (LTE) or other protocol. In addition a GPS sensor may be present in certain embodiments (not necessarily shown in block diagram). Other wireless and/or wired communications such as, e.g., but not limited to, receipt or transmission of radio signals, e.g., AM/FM, Wi-Fi, WiMAX, etc., and other signals may also be provided, on a local area, and/or a wide area basis. In addition, via an exemplary WLAN transceiver, local wireless communications can also be realized.

Further referring to FIG. 26, shown is a block diagram of another example system with which embodiments may be used. In the illustration of exemplary systems herein, some communications devices may be mobile, and/or portable, and/or, low-power system(s) such as, e.g., but not limited to, a tablet computer, 2:1 tablet, phablet, a smartphone, a laptop, a notebook, a portable computer, a personal computer, a telephony device, a cellphone, an ultrabook, a GOOGLE CHROME book, etc, a thick client, a fat client, a thin client, and/or convertible and/or standalone, and/or desktop and/or tablet system. As illustrated, a system on a chip (SoC) can also be used and may be configured to operate as a system processor, and/or an application processor for the device.

A variety of devices may couple to an exemplary SoC. In the illustration shown, a memory subsystem may include an exemplary flash memory and/or a DRAM coupled to a SoC, and/or processor and/or controller, and/or microcontroller. In addition, a touch panel 1320 is coupled to the SoC, etc. to provide display capability and/or user input via exemplary touch and/or other interface, including, e.g., but not limited to, provision of an actual, and/or virtual keyboard, and/or other input device, which can be alternatively displayed on an exemplary display of an exemplary touch enabled display panel monitor, or other output device or screen, according to exemplary embodiments.

To provide wired network connectivity, SoC or system processor can couple to an exemplary network interface such as, e.g., but not limited to, an exemplary Ethernet interface. A peripheral hub can be coupled to SoC or system processor, in some embodiments, to enable interfacing with various peripheral devices, such as may be coupled to system by any of various ports and/or other connectors. Various other output devices can include any of various indicators such as, e.g., but not limited to, display interfaces, LEDs, LCDs, etc., interfaces, command line interfaces, graphical user interfaces, etc.

In addition to internal power management circuitry and functionality optionally provided in some embodiments, within SoC or system processor, or a PMIC can be coupled to exemplary SoC or system processor embodiments to provide exemplary platform-based power management, e.g., based on whether the system is powered by a battery, or AC power, via an AC adapter, and/or uninterruptible power supply or other power source, in an exemplary embodiment. In addition to this power source-based power management, PMIC may further perform platform power management activities based on environmental and usage conditions in some embodiments. Still further, PMIC may communicate control and status information to SoC or system processor or controller to cause various power management actions within SoC or system processor, in exemplary embodiments.

Still referring to FIG. 26, to provide for exemplary communication functions, such as, e.g., but not limited to, wired capabilities, and/or wireless capabilities, a communication interface, such as, e.g., a WLAN unit can be coupled to SoC or system processor and/or in turn to an exemplary antenna. In various implementations, WLAN unit or other communications devices may provide for communication according to one or more wireless and/or wired communications protocols, as described herein, and as would be apparent to those skilled in the relevant art.

As further describing FIG. 26 in illustrative embodiments, a plurality of sensors (not shown) may couple to SoC and/or the system processor. These sensor(s) may include various accelerometer, environmental and other sensors, including, e.g., but not limited to, user gesture sensors, range finders, location based sensors, gyroscopic, touch, ultrasonic, and/or other well known sensors. Finally, an audio codec, and/or an analog to digital converter, and/or digital to analog converter, can be coupled to SoC or system processor to provide an interface to an exemplary audio input and/or output device. Of course, as will be understood to those skilled in the relevant art, such examples are intended merely as way of example, but not limitation, and that whether shown or not shown, discussed, or not discussed, are intended still to potentially fall with this particular implementations as described in the exemplary figures including FIG. 26, however many variations and alternatives are possible within the scope of the claims as set forth below.

The exemplary embodiment of the present invention makes reference to wired, or wireless networks. Wired networks include any of a wide variety of well known means for coupling voice and data communications devices together. A brief discussion of various exemplary wireless network technologies that may be used to implement the embodiments of the present invention now are discussed. The examples are non-limited. Exemplary wireless network types may include, e.g., but not limited to, code division multiple access (CDMA), spread spectrum wireless, orthogonal frequency division multiplexing (OFDM), 1G, 2G, 3G, 4G, 5G, 6G, n-G (any future wireless standard), next generation (NG), wireless, Bluetooth, Infrared Data Association (IrDA), shared wireless access protocol (SWAP), “wireless fidelity” (Wi-Fi), WIMAX, and other IEEE standard 802.11-compliant wireless local area network (LAN), 802.16-compliant wide area network (WAN), and ultrawideband (UWB), etc.

Bluetooth is a wireless technology promising to unify several wireless technologies for use in low power radio frequency (RF) networks.

IrDA is a standard method for devices to communicate using infrared light pulses, as promulgated by the Infrared Data Association from which the standard gets its name. Since IrDA devices use infrared light, they may depend on being in line of sight with each other.

The exemplary embodiments of the present invention may make reference to WLANs. Examples of a WLAN may include a shared wireless access protocol (SWAP) developed by Home radio frequency (HomeRF), and wireless fidelity (Wi-Fi), a derivative of IEEE 802.11, advocated by the wireless Ethernet compatibility alliance (WECA). The IEEE 802.11 wireless LAN standard refers to various technologies that adhere to one or more of various wireless LAN standards. An IEEE 802.11 compliant wireless LAN may comply with any of one or more of the various IEEE 802.11 wireless LAN standards including, e.g., but not limited to, wireless LANs compliant with IEEE std. 802.11a, b, d, g, n, etc. such as, e.g., but not limited to, IEEE std. 802.11 a, b, d, g, n, (including, e.g., but not limited to IEEE 802.11g-2003, etc.), IEEE 802.16, Wi-MAX, etc.

Wide area networks (WANs) allow extending of computer networks over large distances, connecting or coupling remote branch offices to data centers and to other branch offices, and delivery of applications and services required to perform business functions. When entities like companies or government agencies extend networks over greater distances and sometimes across multiple carriers' networks, the entities can face operational challenges including, e.g., but not limited to, latency, network congestion, jitter, packet loss, and/or even service outages, etc. Modern communications related applications such as, e.g., but not limited to, voice over internet protocol (VoIP) calling, videoconferencing, streaming media, and/or virtualized applications and/or desktops, etc., can require low latency. Bandwidth requirements are also continually increasing, especially for applications featuring high-definition video, and the like. Expanding WAN capability can be expensive and difficult with corresponding difficulties related to network management and troubleshooting.

The Applicant provides reference to the following references, the contents of all of which are incorporated herein by reference in their entireties. First, Applicant references prior US Patent Application published first as US 2012/0073574 Mar. 29, 2012, and later as U.S. Pat. No. 8,573,207, issued Nov. 5, 2013, the contents of all of which is incorporated herein by reference in its entirety, as well as acknowledging the contributions of student technician's contributions set forth in “Final Report—BME 4925W—Capstone Project Lab, Group #7, Continuous Respiration Monitor,” including authors Camille Roberts, Abbey Thorpe, Topher Copeland, Aisling Casey, and Srineil Nizambad, and Mentor: Jason Zara, under the direction of Applicant Inventor Dr. Guillermo Gutierrez; Submitted: May 3, 2017.

### INCORPORATED NONPATENT REFERENCES

[1] Couvetier, M. “GWU Hospital Biomedical Engineering Department Device Specifications Interview.” Personal Interview. 3 Nov. 2016.

[2] Gutierrez, G., A. Das, G. Ballarino, A. Beyzaei-Arani, H. Turkan, M. Wulf-Gutierrez, K. Rider, H. Kaya, and R. Amdur. Decreased respiratory rate variability during mechanical ventilation is associated with increased mortality. Intensive Care Med. 39:1359-1367, 2013.

[3] Gutierrez, G., J. Williams, G. A. Alrehaili, A. McLean, R. Pirouz, R. Amdur, V. Jain, J. Ahari, A. Bawa, and S. Kimbro. Respiratory rate variability in sleeping adults without obstructive sleep apnea. Physiol. Rep. 4:1-9, 2016.

[4] Gutierrez, G., G. J. Ballarino, H. Turkan, J. Abril, L. D. L. Cruz, C. Edsall, B. George, S. Gutierrez, V. Jha, J. Ahari. “Automatic detection of patient-ventilator asynchrony by spectral analysis of airway flow.” Critical Care. 15: 1-10, 2011.

[5] Helou, G., M. Moore. “Respiration Monitor Interface Consultation with GWU

Hospital Clinicians.” Personal interview. 10 Nov. 2016.

[6] https://arduinodiy.wordpress.com/2012/03/19/serial-connection-for-your-arduino-atmega/Accessed 3 May 2017.

[7] https://developer.mbed.org/users/chris215/code/4dGENIE/ Accessed 4 May 2017

[8] https://measuringu.com/onep/. Accessed 4 May 2017.

[9] http://provenprocess.com/engineering-services/early-stage-technology-development. Accessed 22 Nov. 2016.

[10] http://statisticcafe.blogspot.com/2011/05/how-to-use-likeert-scale-in-statistical.html Accessed 4 May 2017

[11] Maquet Critical Care AB. SERVO-i/SERVO-s, Computer interface emulator reference manual, Revision 07. 2008.

[12] Payen, J., G. Chanques, J. Mantz, C. Hercule, I. Auriant, J. Leguillo, M. Binhas, C. Genty, C. Rolland, and J. Bosson. Current practices in sedation and analgesia for mechanically ventilated critically ill patients. Anesthesiology. 106:687-695, 2007.

[13] Sullivan G M, Artino A R. “Analyzing and Interpreting Data From Likert-Type Scales.” Journal of Graduate Medical Education, Revision 2013. Accessed 03, 2017.

[14] Wysocki, M., C. Cracco, A. Teixeira, A. Mercat, J L. Diehlm Y. Lefort, J P. Derenne, and T. Similowski. Reduced breathing variability as a predictor of unsuccessful patient separation from mechanical ventilation. Crit. Care Med. 8:2076-2083, 2006.

[15] 4D Systems Application Note. “Workshop 4 Visi User Guide.” Published 22 Dec. 2013. Revision 1.3. http://www.4dsystems.com.au/product/4D Workshop_4_IDE

[16] 4D Systems Application Note. “Visi-Genie Program Destination.” Published 7 Nov. 2015. Revision 1.01. http://www.4dsystems.com.au/appnote/4D-AN-00202/

Uses of conjunction language such as the word or, and/or, and, and the like, are intended herein to include the logical or interpretation, rather than an exclusive or interpretation. Thus, any use of or, can mean either alternative individually, and/or both alternatives. Further, where a plurality of alternatives are listed, any one or more of the alternatives can be considered, thus if a list of five alternatives are listed, any one or more, or combinations of any of the five alternatives, including all five alternatives, can also be considered within the scope of protection.

All examples and conditional language recited herein are intended for pedagogical purposes to aid the reader in understanding the principles of the disclosure and the concepts contributed by the inventor to furthering the art, and are to be construed as being without limitation to such specifically recited examples and conditions. Moreover, all statements herein reciting principles, aspects, and embodiments, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

What is claimed is:

1. A computerized method of detecting patient-ventilator asynchrony comprising:
electronically obtaining, by at least one processor, a flow or pressure signal representative of respiratory movement of a patient on ventilatory support of a mechanical ventilator, captured by at least one respiratory sensor;
electronically modifying, by the at least one processor, the flow or pressure signal comprising:
taking at least a portion of the flow or pressure signal; and
electronically transforming said at least said portion of the flow or pressure signal in accordance with a Fourier transform to obtain a frequency spectrum of said at least said portion of the flow or pressure signal;
electronically obtaining, by the at least one processor, a measure of spectral organization wherein said spectral organization comprises a spectral pattern of recurrent peaks of the obtained frequency spectrum;
wherein said electronically obtaining the measure of spectral organization comprises:
electronically obtaining, by the at least one processor, a ratio of a harmonic peak amplitude to an amplitude of a direct current (DC) component of said at least said portion of the flow or pressure signal,
said electronically obtaining said ratio comprising:
electronically dividing, by the at least one processor, the harmonic peak amplitude, by the amplitude of the DC component of said at least said portion of the flow or pressure signal; and
electronically providing, by the at least one processor, at least one or more of:
an instruction,
an indication representative of at least one of:
an output, or
an output signal, representative of the measure, or
wherein said electronically providing comprises at least one or more of:
providing autonomous interventions based on machine learning analysis of continuous mechanical ventilator monitored data comprising a plurality of continuous epoch data in combination with identification of interventions that resulted in positive outcomes;
electronically providing instructions to perform an intervention; or
electronically providing a graphical user interface, by the at least one processor, displaying at least one or more of:
said instruction, or
said indication,
wherein said indication comprises a color indicator, and wherein said providing said graphical user interface comprising at least one of:
electronically displaying, by the at least one processor, an indicator of a value of said at least one of said output or said output signal representative of the measure;
electronically displaying, by the at least one processor, a color indicator of a value of said at least one of said output or said output signal representative of the measure; or
electronically displaying, by the at least one processor, a trend indicator of a range of historical values for a period of time of said at least one of said output or said output signal representative of the measure.

2. The method of detecting patient-ventilator asynchrony according to claim 1,
wherein the electronically obtaining a measure of spectral organization comprises obtaining, by the at least one processor, a coherence function of the obtained frequency spectrum.

3. The method of detecting patient-ventilator asynchrony according to claim 1,
wherein said electronically providing said at least one of said output or said output signal comprises at least one of:
electronically displaying or providing, by the at least one processor, a value corresponding to the measure;
electronically displaying or electronically providing, by the at least one processor, an indication associated with a value of the measure; or
electronically displaying or electronically providing, by the at least one processor, at least one alert associated with a value of the measure.

4. The method of detecting patient-ventilator asynchrony according to claim 1,
wherein said electronically providing said at least one of said output or said output signal comprises at least one of:
electronically displaying, or electronically providing, by the at least one processor, the frequency spectrum of the signal;
electronically displaying or electronically providing, by the at least one processor, an indication associated with the frequency spectrum of the signal; or
electronically displaying or electronically providing, by the at least one processor, at least one alert associated with the frequency spectrum of the signal.

5. The method of detecting patient-ventilator asynchrony according to claim 1,
wherein said electronically providing said at least one of said output or said output signal comprises at least one of:
electronically providing an indication to adjust, by the at least one processor, the mechanical ventilator to adjust patient-ventilator asynchrony in response to at least one of the output signal, or the measure of spectral organization;
electronically providing an indication to adjust, by the at least one processor, the mechanical ventilator to increase patient-ventilator asynchrony in response to at least one of the output signal, or the measure of spectral organization; or
electronically providing an indication to adjust, by the at least one processor, the mechanical ventilator to decrease patient-ventilator asynchrony in response to at least one of the output signal, or the measure of spectral organization.

6. The method of detecting patient-ventilator asynchrony according to claim 1,
wherein the flow or pressure signal represents at least one of:
airway flow,
airway pressure,
tidal volume,
measurements of flow made with at least one thermistor placed near at least one of a nose, or a mouth of the patient, or
measurement of at least one of rib or abdominal expansion movement of the patient made during respiration by the patient on the ventilator.

7. The method according to claim 1, wherein the harmonic peak amplitude comprises at least one of:
an amplitude height of a first harmonic peak of the frequency spectrum of the flow or pressure signal; or
an area under a first harmonic peak of the frequency spectrum of the flow or pressure signal.

8. The method according to claim 1, wherein said at least said portion comprises at least one of:
an inspiratory portion of the flow signal;
an expiratory portion;
or at least a portion of said pressure signal.

9. The method according to claim 1, wherein said at least said portion comprises at least one of:
an expiratory portion, representing patient effort; or
at least a portion of the amplitude of the at least a portion of flow signal or pressure signal.

10. The method according to claim 1, further comprising at least one of:
electronically providing, by the at least one processor, at least one of an alert or an indication to adjust sedation;
electronically providing, by the at least one processor, at least one of an alert or an indication to adjust use of at least one paralytic agent; or
electronically providing, by the at least one processor, at least one of an alert or an indication to adjust ventilatory support.

11. A system for detecting patient-ventilator asynchrony comprising:
at least one sensor operatively coupled to an air output of a mechanical ventilator for providing a sensor output representative of a characteristic of the output flow or pressure;
at least one data acquisition unit operatively coupled to said at least one sensor to collect time dependent data representative of the sensor output flow or pressure to obtain a time dependent signal representative of respiratory movement of a patient;
at least one processor operatively coupled to said at least one data acquisition unit, wherein said at least one processor is configured to:
take at least a portion of the flow or pressure,
transform said at least said portion of the flow or pressure comprising wherein said at least one processor is configured to:
perform a Fourier transform of the collected time dependent data to determine a measure of spectral organization of the transformation of the collected time dependent data,
wherein to determine the measure of spectral organization of the transformation comprises wherein said at least one processor is configured to:
determine a ratio of a harmonic peak amplitude to an amplitude of a direct current (DC) component of said at least said portion of the flow or pressure of the measure of spectral organization of the transformation of the collected time dependent data, and wherein said at least one processor is configured to:
provide, or output data indicative of a signal representative of the measure of spectral organization,
wherein said at least one processor configured to provide comprises wherein said at least one processor is configured to:
provide a graphical user interface, by the at least one processor, configured to provide a color-coded indicator, output or signal, and at least one of:
display an indicator of a value of said at least one of said output or said output signal representative of the measure;
display a color indicator of a value of said at least one of said output or said output signal representative of the measure; or
display a trend indicator of a range of historical values for a period of time of said at least one of said output or said output signal representative of the measure.

12. The system for detecting patient-ventilator asynchrony according to claim 11, wherein the measure of spectral organization comprises:
wherein said at least one processor is configured to determine a coherence function of the transformation of the collected data.

13. The system for detecting patient-ventilator asynchrony according to claim 11, wherein the at least one sensor comprises at least one:
a flow sensor;
a respiratory sensor;
a pressure sensor;
a mechanical ventilator; or
a ventilator.

14. The system for detecting patient-ventilator asynchrony according to claim 11, further comprising at least one of:
a heart monitor operatively coupled to provide heart rate data to the data acquisition unit; or a patient monitor operatively coupled to a patient to provide patient data to the data acquisition unit.

15. The monitoring system according to claim 11, further comprising at least one of:
a mechanical ventilator;
a respiratory monitor;
a positive pressure mechanical ventilator;
an intensive care unit (ICU) monitor; or
a hemodynamic monitor.

16. A computerized method of detecting patient-ventilator asynchrony comprising:
electronically obtaining, by at least one processor, a flow or pressure signal representative of respiratory movement of a patient on ventilatory support of a mechanical ventilator, captured by at least one respiratory sensor;
electronically modifying, by the at least one processor, the flow or pressure signal comprising:
taking at least a portion of the flow or pressure signal; and
electronically transforming said at least said portion of the flow or pressure signal in accordance with a Fourier transform to obtain a frequency spectrum of said at least said portion of the flow or pressure signal;
electronically obtaining, by the at least one processor, a measure of spectral organization wherein said spectral organization comprises a spectral pattern of recurrent peaks of the obtained frequency spectrum;
wherein said electronically obtaining the measure of spectral organization comprises:
electronically obtaining, by the at least one processor, a ratio of a harmonic peak amplitude to an amplitude of a direct current (DC) component of said at least said portion of the flow or pressure signal,
wherein said obtaining said ratio comprises:
electronically dividing, by the at least one processor, the harmonic peak amplitude, by the amplitude of the DC component of said at least said portion of the flow or pressure signal;
electronically providing, by the at least one processor, at least one of a color-coded indicator, a color-coded output, or a color-coded output signal, representative of the measure,
wherein said electronically providing further comprises at least one of:
electronically providing, by the at least one processor, at least one of an alert or an indication to adjust sedation;
electronically providing, by the at least one processor, at least one of an alert or an indication to adjust use of at least one paralytic agent;
electronically providing, by the at least one processor, at least one of an alert or an indication to adjust ventilatory support;
electronically providing, by the at least one processor, at least one of an alert or indication to adjust a dose of sedative;
electronically providing, by the at least one processor, at least one of an alert or indication to discontinue sedation;
electronically providing, by the at least one processor, at least one of an alert or indication to increase sedation;
electronically providing, by the at least one processor, at least one of an alert or indication to decrease sedation;
electronically providing, by the at least one processor, at least one of an alert or indication to adjust a neuromuscular blocking therapeutic measure;
electronically providing, by the at least one processor, at least one of an alert or indication to adjust a paralytic agent;
electronically providing, by the at least one processor, at least one of an alert or indication to adjust the mechanical ventilator; or
electronically providing a graphical user interface, by the at least one processor, and at least one of:
electronically displaying, by the at least one processor, an indicator of a value of said at least one of said output or said output signal representative of the measure;
electronically displaying, by the at least one processor, a color indicator of a value of said at least one of said output or said output signal representative of the measure; or
electronically displaying, by the at least one processor, a trend indicator of a range of historical values for a period of time of said at least one of said output or said output signal representative of the measure.

17. The method according to claim 1, wherein said color indicator of said value of said at least one of said output or said output signal representative of the measure comprises:
color-coded zones corresponding to ranges of said value, and
wherein a yellow color-coded zone corresponds to a patient being under ideal synchrony state with the ventilator,
wherein a red color-coded zone corresponds to a patient being in an over ideal synchrony state with the ventilator, and
wherein a green color-coded zone corresponds to a patient being in an ideal synchrony state with the ventilator.

18. The system according to claim 11, wherein said color-coded indicator, output, or signal of said value of said at least one of said output or said output signal representative of the measure comprises:
color-coded zones corresponding to ranges of said value, and
wherein a yellow color-coded zone corresponds to a patient being under ideal synchrony state with the ventilator,
wherein a red color-coded zone corresponds to a patient being in an over ideal synchrony state with the ventilator, and
wherein a green color-coded zone corresponds to a patient being in an ideal synchrony state with the ventilator.

19. The method according to claim 16, wherein said at least one of said color-coded indicator, said color-coded output, or said color-coded output signal of said value of said at least one of said output or said output signal representative of the measure comprises:
color-coded zones corresponding to ranges of said value, and
wherein a yellow color-coded zone corresponds to a patient being under ideal synchrony state with the ventilator,
wherein a red color-coded zone corresponds to a patient being in an over ideal synchrony state with the ventilator, and
wherein a green color-coded zone corresponds to a patient being in an ideal synchrony state with the ventilator.

20. The method according to claim 1, comprising using artificial intelligence or machine learning to predict interventions predicted to result in positive outcomes, based on analysis of a large number of epochs, captured from a monitor of a mechanical ventilator, wherein said monitor continuously monitors said mechanical ventilator and captures and transfers epochs of data.
